SEQUENCE LISTING (529 sequences)

SEQ ID NO: 1 - DNA, 23 bp, Artificial Sequence
  Synthetic
ccaacgctgggctgcacgctaca

SEQ ID NO: 2 - DNA, 25 bp, Artificial Sequence
  Synthetic
acggacgcggagccaccaggccccv

SEQ ID NO: 3 - DNA, 23 bp, Artificial Sequence
  Synthetic
cgcgccgaggtcaccaggccccv

SEQ ID NO: 4 - DNA, 40 bp, Artificial Sequence
  Synthetic
gcagggggcctggtgggtagcgtgcagcccagcgttggcg

SEQ ID NO: 5 - DNA, 40 bp, Artificial Sequence
  Synthetic
gcagggggcctggtgagtagcgtgcagcccagcgttggcg

SEQ ID NO: 6 - DNA, 22 bp, Artificial Sequence
  Synthetic
gccgccttcgccaaccactcct

SEQ ID NO: 7 - DNA, 26 bp, Artificial Sequence
  Synthetic
acggacgcggagggtgggtgatgggv

SEQ ID NO: 8 - DNA, 25 bp, Artificial Sequence
  Synthetic
cgcgccgaggagtgggtgatgggcv

SEQ ID NO: 9 - DNA, 41 bp, Artificial Sequence
  Synthetic
ttctgcccatcacccaccggagtggttggcgaaggcggcac

SEQ ID NO: 10 - DNA, 41 bp, Artificial Sequence
  Synthetic
ttctgcccatcacccactggagtggttggcgaaggcggcac

SEQ ID NO: 11 - DNA, 21 bp, Artificial Sequence
  Synthetic
ccggggctgtccagtgggcat

SEQ ID NO: 12 - DNA, 24 bp, Artificial Sequence
  Synthetic
cgcgccgaggcagtgggcaccgav

SEQ ID NO: 13 - DNA, 29 bp, Artificial Sequence
  Synthetic
acggacgcggagccgagaagctgaagtgv

SEQ ID NO: 14 - DNA, 51 bp, Artificial Sequence
  Synthetic
gcagcacttcagcttctcggtgcccactgtgcccactggacagccccggcc

SEQ ID NO: 15 - DNA, 42 bp, Artificial Sequence
  Synthetic
gcagcacttcagcttctcggtgcccactggacagccccggcc

SEQ ID NO: 16 - DNA, 28 bp, Artificial Sequence
  Synthetic
ctccctgctgcagcacttcagcttctct

SEQ ID NO: 17 - DNA, 24 bp, Artificial Sequence
  Synthetic
cgcgccgaggggtgcccactgtgv

SEQ ID NO: 18 - DNA, 26 bp, Artificial Sequence
  Synthetic
acggacgcggagggtgcccactggav

SEQ ID NO: 19 - DNA, 55 bp, Artificial Sequence
  Synthetic
gctgtccagtgggcacagtgggcaccgagaagctgaagtgctgcagcagggaggt

SEQ ID NO: 20 - DNA, 46 bp, Artificial Sequence
  Synthetic
gctgtccagtgggcaccgagaagctgaagtgctgcagcagggaggt

SEQ ID NO: 21 - DNA, 35 bp, Artificial Sequence
  Synthetic
gaaccctgagagcagcttcaatgatgagaacctga

SEQ ID NO: 22 - DNA, 27 bp, Artificial Sequence
  Synthetic
acggacgcggagcgcatagtggtggcv

SEQ ID NO: 23 - DNA, 26 bp, Artificial Sequence
  Synthetic
cgcgccgaggtgcatagtggtggctv

SEQ ID NO: 24 - DNA, 55 bp, Artificial Sequence
  Synthetic
ggtcagccaccactatgcgcaggttctcatcattgaagctgctctcagggttccc

SEQ ID NO: 25 - DNA, 55 bp, Artificial Sequence
  Synthetic
ggtcagccaccactatgcacaggttctcatcattgaagctgctctcagggttccc

SEQ ID NO: 26 - DNA, 30 bp, Artificial Sequence
  Synthetic
ccaccatggtgtctttgctttcctggtgat

SEQ ID NO: 27 - DNA, 27 bp, Artificial Sequence
  Synthetic
acggacgcggaggcccatccccctatv

SEQ ID NO: 28 - DNA, 25 bp, Artificial Sequence
  Synthetic
cgcgccgaggccccatccccctatv

SEQ ID NO: 29 - DNA, 49 bp, Artificial Sequence
  Synthetic
gctcatagggggatgggctcaccaggaaagcaaagacaccatggtggct

SEQ ID NO: 30 - DNA, 49 bp, Artificial Sequence
  Synthetic
gctcatagggggatggggtcaccaggaaagcaaagacaccatggtggct

SEQ ID NO: 31 - DNA, 29 bp, Artificial Sequence
  Synthetic
cccagctggatgagctgctaactgagcat

SEQ ID NO: 32 - DNA, 26 bp, Artificial Sequence
  Synthetic
cgcgccgaggcaggatgacctgggav

SEQ ID NO: 33 - DNA, 27 bp, Artificial Sequence
  Synthetic
acggacgcggagcggatgacctgggav

SEQ ID NO: 34 - DNA, 50 bp, Artificial Sequence
  Synthetic
ctgggtcccaggtcatcctgtgctcagttagcagctcatccagctgggtc

SEQ ID NO: 35 - DNA, 49 bp, Artificial Sequence
  Synthetic
ctgggtcccaggtcatccgtgctcagttagcagctcatccagctgggtc

SEQ ID NO: 36 - DNA, 24 bp, Artificial Sequence
  Synthetic
cttacccgcatctcccacccccat

SEQ ID NO: 37 - DNA, 26 bp, Artificial Sequence
  Synthetic
acggacgcggagggacgcccctttcv

SEQ ID NO: 38 - DNA, 25 bp, Artificial Sequence
  Synthetic
cgcgccgaggagacgcccctttcgv

SEQ ID NO: 39 - DNA, 43 bp, Artificial Sequence
  Synthetic
ggggcgaaaggggcgtcctgggggtgggagatgcgggtaaggg

SEQ ID NO: 40 - DNA, 43 bp, Artificial Sequence
  Synthetic
ggggcgaaaggggcgtcttgggggtgggagatgcgggtaaggg

SEQ ID NO: 41 - DNA, 25 bp, Artificial Sequence
  Synthetic
cctgggcaagaagtcgctggagcat

SEQ ID NO: 42 - DNA, 25 bp, Artificial Sequence
  Synthetic
cgcgccgagggtgggtgaccgaggv

SEQ ID NO: 43 - DNA, 26 bp, Artificial Sequence
  Synthetic
acggacgcggagggggtgaccgaggv

SEQ ID NO: 44 - DNA, 45 bp, Artificial Sequence
  Synthetic
ggcctcctcggtcacccactgctccagcgacttcttgcccaggcc

SEQ ID NO: 45 - DNA, 44 bp, Artificial Sequence
  Synthetic
ggcctcctcggtcacccctgctccagcgacttcttgcccaggcc

SEQ ID NO: 46 - DNA, 28 bp, Artificial Sequence
  Synthetic
cctggggcctcctgctcatgatcctact

SEQ ID NO: 47 - DNA, 25 bp, Artificial Sequence
  Synthetic
cgcgccgaggatccggatgtgcagv

SEQ ID NO: 48 - DNA, 27 bp, Artificial Sequence
  Synthetic
acggacgcggagctccggatgtgcagv

SEQ ID NO: 49 - DNA, 47 bp, Artificial Sequence
  Synthetic
cacgctgcacatccggatgtaggatcatgagcaggaggccccaggcc

SEQ ID NO: 50 - DNA, 47 bp, Artificial Sequence
  Synthetic
cacgctgcacatccggaggtaggatcatgagcaggaggccccaggcc

SEQ ID NO: 51 - DNA, 22 bp, Artificial Sequence
  Synthetic
gccgccttcgccaaccactccc

SEQ ID NO: 52 - DNA, 26 bp, Artificial Sequence
  Synthetic
acggacgcggagggtgggtgatgggv

SEQ ID NO: 53 - DNA, 25 bp, Artificial Sequence
  Synthetic
cgcgccgaggtgtgggtgatgggcv

SEQ ID NO: 54 - DNA, 41 bp, Artificial Sequence; identical to SEQ ID NO: 9
  Synthetic

SEQ ID NO: 55 - DNA, 41 bp, Artificial Sequence
  Synthetic
ttctgcccatcacccacaggagtggttggcgaaggcggcac

SEQ ID NO: 56 - DNA, 31 bp, Artificial Sequence
  Synthetic
gccaccatggtgtctttgctttcctggtgat

SEQ ID NO: 57 - DNA, 26 bp, Artificial Sequence
  Synthetic
cgcgccgaggccccatccccctatgv

SEQ ID NO: 58 - DNA, 27 bp, Artificial Sequence
  Synthetic
acggacgcggaggcccatccccctatv

SEQ ID NO: 59 - DNA, 51 bp, Artificial Sequence
  Synthetic
agctcatagggggatggggtcaccaggaaagcaaagacaccatggtggctg

SEQ ID NO: 60 - DNA, 51 bp, Artificial Sequence
  Synthetic
agctcatagggggatgggctcaccaggaaagcaaagacaccatggtggctg

SEQ ID NO: 61 - DNA, 24 bp, Artificial Sequence
  Synthetic
gctgggctgggtcccaggtcatct

SEQ ID NO: 62 - DNA, 28 bp, Artificial Sequence
  Synthetic
cgcgccgaggctgtgctcagttagcagv

SEQ ID NO: 63 - DNA, 29 bp, Artificial Sequence
  Synthetic
acggacgcggagcgtgctcagttagcagv

SEQ ID NO: 64 - DNA, 47 bp, Artificial Sequence
  Synthetic
atgagctgctaactgagcacaggatgacctgggacccagcccagccc

SEQ ID NO: 65 - DNA, 46 bp, Artificial Sequence
  Synthetic
atgagctgctaactgagcacggatgacctgggacccagcccagccc

SEQ ID NO: 66 - DNA, 25 bp, Artificial Sequence
  Synthetic
ccttacccgcatctcccacccccat

SEQ ID NO: 67 - DNA, 25 bp, Artificial Sequence
  Synthetic
cgcgccgaggagacgcccctttcgv

SEQ ID NO: 68 - DNA, 26 bp, Artificial Sequence
  Synthetic
acggacgcggagggacgcccctttcv

SEQ ID NO: 69 - DNA, 44 bp, Artificial Sequence
  Synthetic
ggggcgaaaggggcgtcttgggggtgggagatgcgggtaagggg

SEQ ID NO: 70 - DNA, 44 bp, Artificial Sequence
  Synthetic
ggggcgaaaggggcgtcctgggggtgggagatgcgggtaagggg

SEQ ID NO: 71 - DNA, 22 bp, Artificial Sequence
  Synthetic
caggcggcctcctcggtcacct

SEQ ID NO: 72 - DNA, 25 bp, Artificial Sequence
  Synthetic
cgcgccgaggcactgctccagcgav

SEQ ID NO: 73 - DNA, 26 bp, Artificial Sequence
  Synthetic
acggacgcggagcctgctccagcgav

SEQ ID NO: 74 - DNA, 42 bp, Artificial Sequence
  Synthetic
agaagtcgctggagcagtgggtgaccgaggaggccgcctgcc

SEQ ID NO: 75 - DNA, 41 bp, Artificial Sequence
  Synthetic
agaagtcgctggagcaggggtgaccgaggaggccgcctgcc

SEQ ID NO: 76 - DNA, 23 bp, Artificial Sequence
  Synthetic
gggctcacgctgcacatccggac

SEQ ID NO: 77 - DNA, 29 bp, Artificial Sequence
  Synthetic
cgcgccgaggtgtaggatcatgagcaggv

SEQ ID NO: 78 - DNA, 30 bp, Artificial Sequence
  Synthetic
acggacgcggagggtaggatcatgagcagv

SEQ ID NO: 79 - DNA, 46 bp, Artificial Sequence
  Synthetic
gcctcctgctcatgatcctacatccggatgtgcagcgtgagcccat

SEQ ID NO: 80 - DNA, 46 bp, Artificial Sequence
  Synthetic
gcctcctgctcatgatcctacctccggatgtgcagcgtgagcccat

SEQ ID NO: 81 - DNA, 22 bp, Artificial Sequence
  Synthetic
gcagtggcagggggcctggtgt

SEQ ID NO: 82 - DNA, 26 bp, Artificial Sequence
  Synthetic
acggacgcggagggtagcgtgcagcv

SEQ ID NO: 83 - DNA, 25 bp, Artificial Sequence
  Synthetic
cgcgccgaggagtagcgtgcagccv

SEQ ID NO: 84 - DNA, 41 bp, Artificial Sequence
  Synthetic
gctgggctgcacgctacccaccaggccccctgccactgccc

SEQ ID NO: 85 - DNA, 41 bp, Artificial Sequence
  Synthetic
gctgggctgcacgctactcaccaggccccctgccactgccc

SEQ ID NO: 86 - DNA, 23 bp, Artificial Sequence
  Synthetic
aggccctgaccctccctctgcat

SEQ ID NO: 87 - DNA, 22 bp, Artificial Sequence
  Synthetic
cgcgccgagggttgcggcgccv

SEQ ID NO: 88 - DNA, 24 bp, Artificial Sequence
  Synthetic
acggacgcggagcttgcggcgccv

SEQ ID NO: 89 - DNA, 39 bp, Artificial Sequence
  Synthetic
aagcggcgccgcaactgcagagggagggtcagggcctct

SEQ ID NO: 90 - DNA, 39 bp, Artificial Sequence
  Synthetic
aagcggcgccgcaagtgcagagggagggtcagggcctct

SEQ ID NO: 91 - DNA, 20 bp, Artificial Sequence
  Synthetic
cgcgaggcgctggtgaccct

SEQ ID NO: 92 - DNA, 26 bp, Artificial Sequence
  Synthetic
acggacgcggagacggcgaggacacv

SEQ ID NO: 93 - DNA, 23 bp, Artificial Sequence
  Synthetic
cgcgccgagggcggcgaggacav

SEQ ID NO: 94 - DNA, 38 bp, Artificial Sequence
  Synthetic
ggcggtgtcctcgccgtgggtcaccagcgcctcgcgca

SEQ ID NO: 95 - DNA, 38 bp, Artificial Sequence
  Synthetic
ggcggtgtcctcgccgcgggtcaccagcgcctcgcgca

SEQ ID NO: 96 - DNA, 28 bp, Artificial Sequence
  Synthetic
tgtgcccatcacccagatcctgggttta

SEQ ID NO: 97 - DNA, 23 bp, Artificial Sequence
  Synthetic
acggacgcggagcgggccgcgtv

SEQ ID NO: 98 - DNA, 22 bp, Artificial Sequence
  Synthetic
cgcgccgaggtgggccgcgttv

SEQ ID NO: 99 - DNA, 44 bp, Artificial Sequence
  Synthetic
tgggaacgcggcccgaaacccaggatctgggtgatgggcacagg

SEQ ID NO: 100 - DNA, 44 bp, Artificial Sequence
  Synthetic
tgggaacgcggcccaaaacccaggatctgggtgatgggcacagg

SEQ ID NO: 101 - DNA, 23 bp, Artificial Sequence
  Synthetic
gcgagcagaggcgcttctccgtt

SEQ ID NO: 102 - DNA, 24 bp, Artificial Sequence
  Synthetic
cgcgccgagggtccaccttgcgcv

SEQ ID NO: 103 - DNA, 26 bp, Artificial Sequence
  Synthetic
acggacgcggagctccaccttgcgcv

SEQ ID NO: 104 - DNA, 41 bp, Artificial Sequence
  Synthetic
agttgcgcaaggtggacacggagaagcgcctctgctcgcgc

SEQ ID NO: 105 - DNA, 41 bp, Artificial Sequence
  Synthetic
agttgcgcaaggtggagacggagaagcgcctctgctcgcgc

SEQ ID NO: 106 - DNA, 23 bp, Artificial Sequence
  Synthetic
tgccgccttcgccaaccactccc

SEQ ID NO: 107 - DNA, 27 bp, Artificial Sequence
  Synthetic
acggacgcggagggtgggtgatgggcv

SEQ ID NO: 108 - DNA, 25 bp, Artificial Sequence
  Synthetic
cgcgccgaggtgtgggtgatgggcv

SEQ ID NO: 109 - DNA, 42 bp, Artificial Sequence
  Synthetic
ttctgcccatcacccaccggagtggttggcgaaggcggcaca

SEQ ID NO: 110 - DNA, 42 bp, Artificial Sequence
  Synthetic
ttctgcccatcacccacaggagtggttggcgaaggcggcaca

SEQ ID NO: 111 - DNA, 27 bp, Artificial Sequence
  Synthetic
caggctgctggacctagctcaggaggt

SEQ ID NO: 112 - DNA, 28 bp, Artificial Sequence
  Synthetic
cgcgccgagggactgaaggaggagtcgv

SEQ ID NO: 113 - DNA, 30 bp, Artificial Sequence
  Synthetic
acggacgcggagaactgaaggaggagtcgv

SEQ ID NO: 114 - DNA, 49 bp, Artificial Sequence
  Synthetic
agcccgactcctccttcagtccctcctgagctaggtccagcagcctgag

SEQ ID NO: 115 - DNA, 49 bp, Artificial Sequence
  Synthetic
agcccgactcctccttcagttcctcctgagctaggtccagcagcctgag

SEQ ID NO: 116 - DNA, 29 bp, Artificial Sequence
  Synthetic
cctgactgaggccttcctggcagagatgt

SEQ ID NO: 117 - DNA, 28 bp, Artificial Sequence
  Synthetic
cgcgccgagggagaaggtgagagtggcv

SEQ ID NO: 118 - DNA, 28 bp, Artificial Sequence
  Synthetic
acggacgcggaggaggtgagagtggctv

SEQ ID NO: 119 - DNA, 54 bp, Artificial Sequence
  Synthetic
cgtggcagccactctcaccttctccatctctgccaggaaggcctcagtcaggtc

SEQ ID NO: 120 - DNA, 51 bp, Artificial Sequence
  Synthetic
cgtggcagccactctcacctccatctctgccaggaaggcctcagtcaggtc

SEQ ID NO: 121 - DNA, 35 bp, Artificial Sequence; identical to SEQ ID NO: 21
  Synthetic

SEQ ID NO: 122 - DNA, 25 bp, Artificial Sequence
  Synthetic
cgcgccgaggcgcatagtggtggcv

SEQ ID NO: 123 - DNA, 28 bp, Artificial Sequence
  Synthetic
acggacgcggagtgcatagtggtggctv

SEQ ID NO: 124 - DNA, 55 bp, Artificial Sequence; identical to SEQ ID NO: 24
  Synthetic

SEQ ID NO: 125 - DNA, 55 bp, Artificial Sequence; identical to SEQ ID NO: 25
  Synthetic

SEQ ID NO: 126 - DNA, 27 bp, Artificial Sequence
  Synthetic
cttccgcttccaccccgaacacttcca

SEQ ID NO: 127 - DNA, 26 bp, Artificial Sequence
  Synthetic
acggacgcggagtggatgcccagggv

SEQ ID NO: 128 - DNA, 24 bp, Artificial Sequence
  Synthetic
cgcgccgaggcggatgcccagggv

SEQ ID NO: 129 - DNA, 45 bp, Artificial Sequence
  Synthetic
gtggccctgggcatccaggaagtgttcggggtggaagcggaaggg

SEQ ID NO: 130 - DNA, 45 bp, Artificial Sequence
  Synthetic
gtggccctgggcatccgggaagtgttcggggtggaagcggaaggg

SEQ ID NO: 131 - DNA, 24 bp, Artificial Sequence
  Synthetic
aggcccaagttgcgcaaggtggat

SEQ ID NO: 132 - DNA, 24 bp, Artificial Sequence
  Synthetic
cgcgccgaggcacggagaagcgcv

SEQ ID NO: 133 - DNA, 26 bp, Artificial Sequence
  Synthetic
acggacgcggaggacggagaagcgcv

SEQ ID NO: 134 - DNA, 42 bp, Artificial Sequence
  Synthetic
agaggcgcttctccgtgtccaccttgcgcaacttgggcctgg

SEQ ID NO: 135 - DNA, 42 bp, Artificial Sequence
  Synthetic
agaggcgcttctccgtctccaccttgcgcaacttgggcctgg

SEQ ID NO: 136 - DNA, 19 bp, Artificial Sequence
  Synthetic
cccgaagcggcgccgcaat

SEQ ID NO: 137 - DNA, 25 bp, Artificial Sequence
  Synthetic
cgcgccgaggctgcagagggagggv

SEQ ID NO: 138 - DNA, 27 bp, Artificial Sequence
  Synthetic
acggacgcggaggtgcagagggagggv

SEQ ID NO: 139 - DNA, 38 bp, Artificial Sequence
  Synthetic
ctgaccctccctctgcagttgcggcgccgcttcgggga

SEQ ID NO: 140 - DNA, 38 bp, Artificial Sequence
  Synthetic
ctgaccctccctctgcacttgcggcgccgcttcgggga

SEQ ID NO: 141 - DNA, 21 bp, Artificial Sequence
  Synthetic
ggtcggcggtgtcctcgccga

SEQ ID NO: 142 - DNA, 26 bp, Artificial Sequence
  Synthetic
acggacgcggagtgggtcaccagcgv

SEQ ID NO: 143 - DNA, 24 bp, Artificial Sequence
  Synthetic
cgcgccgaggcgggtcaccagcgv

SEQ ID NO: 144 - DNA, 39 bp, Artificial Sequence
  Synthetic
gaggcgctggtgacccacggcgaggacaccgccgaccgc

SEQ ID NO: 145 - DNA, 39 bp, Artificial Sequence
  Synthetic
gaggcgctggtgacccgcggcgaggacaccgccgaccgc

SEQ ID NO: 146 - DNA, 22 bp, Artificial Sequence
  Synthetic
cttgccttgggaacgcggccct

SEQ ID NO: 147 - DNA, 27 bp, Artificial Sequence
  Synthetic
cgcgccgagggaaacccaggatctggv

SEQ ID NO: 148 - DNA, 29 bp, Artificial Sequence
  Synthetic
acggacgcggagaaaacccaggatctggv

SEQ ID NO: 149 - DNA, 43 bp, Artificial Sequence
  Synthetic
tcacccagatcctgggtttcgggccgcgttcccaaggcaagca

SEQ ID NO: 150 - DNA, 43 bp, Artificial Sequence
  Synthetic
tcacccagatcctgggttttgggccgcgttcccaaggcaagca

SEQ ID NO: 151 - DNA, 28 bp, Artificial Sequence
  Synthetic
ctttgtgcccttctgcccatcacccact

SEQ ID NO: 152 - DNA, 24 bp, Artificial Sequence
  Synthetic
cgcgccgaggcggagtggttggcv

SEQ ID NO: 153 - DNA, 27 bp, Artificial Sequence
  Synthetic
acggacgcggagaggagtggttggcgv

SEQ ID NO: 154 - DNA, 47 bp, Artificial Sequence
  Synthetic
ccttcgccaaccactccggtgggtgatgggcagaagggcacaaagcg

SEQ ID NO: 155 - DNA, 47 bp, Artificial Sequence
  Synthetic
ccttcgccaaccactcctgtgggtgatgggcagaagggcacaaagcg

SEQ ID NO: 156 - DNA, 28 bp, Artificial Sequence
  Synthetic
cgcagaaagcccgactcctccttcagta

SEQ ID NO: 157 - DNA, 28 bp, Artificial Sequence
  Synthetic
acggacgcggagccctcctgagctaggv

SEQ ID NO: 158 - DNA, 27 bp, Artificial Sequence
  Synthetic
cgcgccgaggtcctcctgagctaggtv

SEQ ID NO: 159 - DNA, 49 bp, Artificial Sequence
  Synthetic
ctggacctagctcaggagggactgaaggaggagtcgggctttctgcgcg

SEQ ID NO: 160 - DNA, 49 bp, Artificial Sequence
  Synthetic
ctggacctagctcaggaggaactgaaggaggagtcgggctttctgcgcg

SEQ ID NO: 161 - DNA, 24 bp, Artificial Sequence
  Synthetic
ccaccgtggcagccactctcaccc

SEQ ID NO: 162 - DNA, 27 bp, Artificial Sequence
  Synthetic
cgcgccgaggttctccatctctgccav

SEQ ID NO: 163 - DNA, 28 bp, Artificial Sequence
  Synthetic
acggacgcggagtccatctctgccaggv

SEQ ID NO: 164 - DNA, 48 bp, Artificial Sequence
  Synthetic
gccttcctggcagagatggagaaggtgagagtggctgccacggtgggg

SEQ ID NO: 165 - DNA, 45 bp, Artificial Sequence
  Synthetic
gccttcctggcagagatggaggtgagagtggctgccacggtgggg

SEQ ID NO: 166 - DNA, 30 bp, Artificial Sequence
  Synthetic
ggcagagaacaggtcagccaccactatgct

SEQ ID NO: 167 - DNA, 29 bp, Artificial Sequence
  Synthetic
cgcgccgagggcaggttctcatcattgav

SEQ ID NO: 168 - DNA, 33 bp, Artificial Sequence
  Synthetic
acggacgcggagacaggttctcatcattgaagv

SEQ ID NO: 169 - DNA, 55 bp, Artificial Sequence
  Synthetic
gcagcttcaatgatgagaacctgcgcatagtggtggctgacctgttctctgccgg

SEQ ID NO: 170 - DNA, 55 bp, Artificial Sequence
  Synthetic
gcagcttcaatgatgagaacctgtgcatagtggtggctgacctgttctctgccgg

SEQ ID NO: 171 - DNA, 27 bp, Artificial Sequence
  Synthetic
gcttcacaaagtggccctgggcatcct

SEQ ID NO: 172 - DNA, 26 bp, Artificial Sequence
  Synthetic
cgcgccgaggaggaagtgttcggggv

SEQ ID NO: 173 - DNA, 27 bp, Artificial Sequence
  Synthetic
acggacgcggaggggaagtgttcgggv

SEQ ID NO: 174 - DNA, 47 bp, Artificial Sequence
  Synthetic
tccaccccgaacacttcctggatgcccagggccactttgtgaagccg

SEQ ID NO: 175 - DNA, 47 bp, Artificial Sequence
  Synthetic
tccaccccgaacacttcccggatgcccagggccactttgtgaagccg

SEQ ID NO: 176 - DNA, 24 bp, Artificial Sequence
  Synthetic
aggcccaagttgcgcaaggtggat

SEQ ID NO: 177 - DNA, 28 bp, Artificial Sequence
  Synthetic
acggacgcggagcayggagaagcgcctv

SEQ ID NO: 178 - DNA, 26 bp, Artificial Sequence
  Synthetic
cgcgccgagggayggagaagcgcctv

SEQ ID NO: 179 - DNA, 44 bp, Artificial Sequence
  Synthetic
gcagaggcgcttctccrtgtccaccttgcgcaacttgggcctgg

SEQ ID NO: 180 - DNA, 44 bp, Artificial Sequence
  Synthetic
gcagaggcgcttctccrtctccaccttgcgcaacttgggcctgg

SEQ ID NO: 181 - DNA, 25 bp, Artificial Sequence
  Synthetic
gcgcgagcagaggcgcttctccrtt

SEQ ID NO: 182 - DNA, 24 bp, Artificial Sequence
  Synthetic
cgcgccgagggtccaccttgcgcv

SEQ ID NO: 183 - DNA, 26 bp, Artificial Sequence
  Synthetic
acggacgcggagctccaccttgcgcv

SEQ ID NO: 184 - DNA, 43 bp, Artificial Sequence
  Synthetic
agttgcgcaaggtggacayggagaagcgcctctgctcgcgcca

SEQ ID NO: 185 - DNA, 43 bp, Artificial Sequence
  Synthetic
agttgcgcaaggtggagayggagaagcgcctctgctcgcgcca

SEQ ID NO: 186 - DNA, 28 bp, Artificial Sequence
  Synthetic
ctttgtgcccttctgcccatcacccact

SEQ ID NO: 187 - DNA, 27 bp, Artificial Sequence
  Synthetic
acggacgcggagcggagtggtyggcgv

SEQ ID NO: 188 - DNA, 26 bp, Artificial Sequence
  Synthetic
cgcgccgaggaggagtggtyggcgav

SEQ ID NO: 189 - DNA, 48 bp, Artificial Sequence
  Synthetic
gccttcgccraccactccggtgggtgatgggcagaagggcacaaagcg

SEQ ID NO: 190 - DNA, 48 bp, Artificial Sequence
  Synthetic
gccttcgccraccactcctgtgggtgatgggcagaagggcacaaagcg

SEQ ID NO: 191 - DNA, 28 bp, Artificial Sequence
  Synthetic
ctttgtgcccttctgcccatcacccaca

SEQ ID NO: 192 - DNA, 25 bp, Artificial Sequence
  Synthetic
cgcgccgaggcggagtggtyggcgv

SEQ ID NO: 193 - DNA, 27 bp, Artificial Sequence
  Synthetic
acggacgcggagtggagtggtyggcgv

SEQ ID NO: 194 - DNA, 47 bp, Artificial Sequence
  Synthetic
ccttcgccraccactccggtgggtgatgggcagaagggcacaaagcg

SEQ ID NO: 195 - DNA, 47 bp, Artificial Sequence
  Synthetic
ccttcgccraccactccagtgggtgatgggcagaagggcacaaagcg

SEQ ID NO: 196 - DNA, 24 bp, Artificial Sequence
  Synthetic
gtgccgccttcgccraccactcct

SEQ ID NO: 197 - DNA, 27 bp, Artificial Sequence
  Synthetic
acggacgcggagggtgggtgatgggcv

SEQ ID NO: 198 - DNA, 25 bp, Artificial Sequence
  Synthetic
cgcgccgaggagtgggtgatgggcv

SEQ ID NO: 199 - DNA, 43 bp, Artificial Sequence
  Synthetic
ttctgcccatcacccaccggagtggtyggcgaaggcggcacaa

SEQ ID NO: 200 - DNA, 43 bp, Artificial Sequence
  Synthetic
ttctgcccatcacccactggagtggtyggcgaaggcggcacaa

SEQ ID NO: 201 - DNA, 25 bp, Artificial Sequence
  Synthetic
cgcggcccraaacccaggatctggt

SEQ ID NO: 202 - DNA, 27 bp, Artificial Sequence
  Synthetic
acggacgcggaggtgatgggcacaggv

SEQ ID NO: 203 - DNA, 26 bp, Artificial Sequence
  Synthetic
cgcgccgaggatgatgggcacaggcv

SEQ ID NO: 204 - DNA, 45 bp, Artificial Sequence
  Synthetic
gcccgcctgtgcccatcacccagatcctgggtttygggccgcgtt

SEQ ID NO: 205 - DNA, 45 bp, Artificial Sequence
  Synthetic
gcccgcctgtgcccatcatccagatcctgggtttygggccgcgtt

SEQ ID NO: 206 - DNA, 20 bp, Artificial Sequence
  Synthetic
cgcccgcctgtgcccatcaa

SEQ ID NO: 207 - DNA, 26 bp, Artificial Sequence
  Synthetic
cgcgccgaggcccagatcctgggttv

SEQ ID NO: 208 - DNA, 30 bp, Artificial Sequence
  Synthetic
acggacgcggagtccagatcctgggtttyv

SEQ ID NO: 209 - DNA, 42 bp, Artificial Sequence
  Synthetic
gcccraaacccaggatctgggtgatgggcacaggcgggcggt

SEQ ID NO: 210 - DNA, 42 bp, Artificial Sequence
  Synthetic
gcccraaacccaggatctggatgatgggcacaggcgggcggt

SEQ ID NO: 211 - DNA, 35 bp, Artificial Sequence; identical to SEQ ID NO: 21
  Synthetic

SEQ ID NO: 212 - DNA, 26 bp, Artificial Sequence
  Synthetic
cgcgccgaggcgcmtagtggtggctv

SEQ ID NO: 213 - DNA, 29 bp, Artificial Sequence
  Synthetic
acggacgcggagtgcmtagtggtggctgv

SEQ ID NO: 214 - DNA, 56 bp, Artificial Sequence
  Synthetic
aggtcagccaccactakgcgcaggttctcatcattgaagctgctctcagggttccc

SEQ ID NO: 215 - DNA, 56 bp, Artificial Sequence
  Synthetic
aggtcagccaccactakgcacaggttctcatcattgaagctgctctcagggttccc

SEQ ID NO: 216 - DNA, 30 bp, Artificial Sequence
  Synthetic
ggcagagaacaggtcagccaccactakgct

SEQ ID NO: 217 - DNA, 31 bp, Artificial Sequence
  Synthetic
acggacgcggaggcaggttctcatcattgav

SEQ ID NO: 218 - DNA, 31 bp, Artificial Sequence
  Synthetic
cgcgccgaggacaggttctcatcattgaagv

SEQ ID NO: 219 - DNA, 55 bp, Artificial Sequence
  Synthetic
gcagcttcaatgatgagaacctgcgcmtagtggtggctgacctgttctctgccgg

SEQ ID NO: 220 - DNA, 55 bp, Artificial Sequence
  Synthetic
gcagcttcaatgatgagaacctgtgcmtagtggtggctgacctgttctctgccgg

SEQ ID NO: 221 - DNA, 30 bp, Artificial Sequence; identical to SEQ ID NO: 216
  Synthetic

SEQ ID NO: 222 - DNA, 31 bp, Artificial Sequence; identical to SEQ ID NO: 217
  Synthetic

SEQ ID NO: 223 - DNA, 31 bp, Artificial Sequence; identical to SEQ ID NO: 218
  Synthetic

SEQ ID NO: 224 - DNA, 55 bp, Artificial Sequence; identical to SEQ ID NO: 219
  Synthetic

SEQ ID NO: 225 - DNA, 55 bp, Artificial Sequence; identical to SEQ ID NO: 220
  Synthetic

SEQ ID NO: 226 - DNA, 35 bp, Artificial Sequence; identical to SEQ ID NO: 21
  Synthetic

SEQ ID NO: 227 - DNA, 28 bp, Artificial Sequence
  Synthetic
acggacgcggagcgcmtagtggtggctv

SEQ ID NO: 228 - DNA, 27 bp, Artificial Sequence
  Synthetic
cgcgccgaggtgcmtagtggtggctgv

SEQ ID NO: 229 - DNA, 56 bp, Artificial Sequence; identical to SEQ ID NO: 214
  Synthetic

SEQ ID NO: 230 - DNA, 56 bp, Artificial Sequence; identical to SEQ ID NO: 215
  Synthetic

SEQ ID NO: 231 - DNA, 22 bp, Artificial Sequence
  Synthetic
cggtcggcsgtgtcctcgccga

SEQ ID NO: 232 - DNA, 27 bp, Artificial Sequence
  Synthetic
acggacgcggagtgggtcaccakcgcv

SEQ ID NO: 233 - DNA, 25 bp, Artificial Sequence
  Synthetic
cgcgccgaggcgggtcaccakcgcv

SEQ ID NO: 234 - DNA, 41 bp, Artificial Sequence
  Synthetic
cgaggcgmtggtgacccacggcgaggacacsgccgaccgcc

SEQ ID NO: 235 - DNA, 41 bp, Artificial Sequence
  Synthetic
cgaggcgmtggtgacccgcggcgaggacacsgccgaccgcc

SEQ ID NO: 236 - DNA, 19 bp, Artificial Sequence
  Synthetic
gcaagaaggagtgtcaggg

SEQ ID NO: 237 - DNA, 18 bp, Artificial Sequence
  Synthetic
aaggctttgcaggcttca

SEQ ID NO: 238 - DNA, 19 bp, Artificial Sequence
  Synthetic
gaatccggtgtcgaagtgg

SEQ ID NO: 239 - DNA, 19 bp, Artificial Sequence
  Synthetic
ctgtggtgaggtgacgagg

SEQ ID NO: 240 - DNA, 19 bp, Artificial Sequence
  Synthetic
gctcggactacggtcatca

SEQ ID NO: 241 - DNA, 17 bp, Artificial Sequence
  Synthetic
ggcccctgcactgtttc

SEQ ID NO: 242 - DNA, 33 bp, Artificial Sequence
  Synthetic
tctagccggttttccggctgagacctcggcgcg

SEQ ID NO: 243 - DNA, 35 bp, Artificial Sequence
  Synthetic
tctagccggttttccggctgagactccgcgtccgt

SEQ ID NO: 244 - DNA, 29 bp, Artificial Sequence
  Synthetic
cccagctggatgagctgctaactgagcat

SEQ ID NO: 245 - DNA, 30 bp, Artificial Sequence
  Synthetic
atgacgtggcagaccaggatgacctgggav

SEQ ID NO: 246 - DNA, 25 bp, Artificial Sequence
  Synthetic
cgcgccgaggcggatgacctgggav

SEQ ID NO: 247 - DNA, 50 bp, Artificial Sequence; identical to SEQ ID NO: 34
  Synthetic

SEQ ID NO: 248 - DNA, 50 bp, Artificial Sequence
  Synthetic
gctgggtcccaggtcatccgtgctcagttagcagctcatccagctgggtc

SEQ ID NO: 249 - DNA, 23 bp, Artificial Sequence
  Synthetic
ccgttggggcgaaaggggcgtca

SEQ ID NO: 250 - DNA, 27 bp, Artificial Sequence
  Synthetic
acggacgcggagttgggggtgggagav

SEQ ID NO: 251 - DNA, 28 bp, Artificial Sequence
  Synthetic
atgacgtggcagacctgggggtgggagv

SEQ ID NO: 252 - DNA, 42 bp, Artificial Sequence
  Synthetic
cgcatctcccacccccaagacgcccctttcgccccaacggtc

SEQ ID NO: 253 - DNA, 42 bp, Artificial Sequence
  Synthetic
cgcatctcccacccccaggacgcccctttcgccccaacggtc

SEQ ID NO: 254 - DNA, 25 bp, Artificial Sequence
  Synthetic
ccatccagggaagagtggcctgttt

SEQ ID NO: 255 - DNA, 28 bp, Artificial Sequence
  Synthetic
acggacgcggagaggaaccctgtgacat

SEQ ID NO: 256 - DNA, 47 bp, Artificial Sequence
  Synthetic
tttgaaatgtcacagggttcctaacaggccactcttccctggatggg

SEQ ID NO: 257 - DNA, 32 bp, Artificial Sequence
  Synthetic
aggagtagccacgctcggtgaggatcttcatt

SEQ ID NO: 258 - DNA, 27 bp, Artificial Sequence
  Synthetic
cgcgccgaggcaggtagtcggtgagat

SEQ ID NO: 259 - DNA, 54 bp, Artificial Sequence
  Synthetic
cgcgatctcaccgactacctgatgaagatcctcaccgagcgtggctactccttc

SEQ ID NO: 260 - DNA, 22 bp, Artificial Sequence
  Synthetic
cccgcgccacccacactgagcc

SEQ ID NO: 261 - DNA, 27 bp, Artificial Sequence
  Synthetic
acggacgcggagttacagcacaggtgc

SEQ ID NO: 262 - DNA, 37 bp, Artificial Sequence
  Synthetic
tctagccggttttccggctgagagtctgccacgtcat

SEQ ID NO: 263 - DNA, 537 bp, Artificial Sequence
  Synthetic
aagttagaagaaccaagactatcttgtcaggggtgtattttgagagtggcagacttttcagtgcctttccattcatgacacttcttgaatctctggcagaaccagccagccgtgttcacagtgtcaaatgaagggatgtctttgattgcttccaggtgttcctcagcaccaccggagggggatgggtgatcagccgaatctttgactcgggctacccatgggacatggtgttcatgacacgctttcagaacatgttgagaaattccctcccaacnccaattgtgacttggttgatggagcgaaagataaacaactggctcaatcatgcaaattacggcttaataccagaagacaggtaaatataatgtgactgccaagggcttttaggaagaaggagcctctgcctgtccagcagcctatacaagccaggcagtaccacagcaacatggctgaatgtgtgggaacacttgatacaaatttgcttgataataacagctaactgttcttaagtactcagaaagtgaaattatgtatttc

SEQ ID NO: 264 - DNA, 19 bp, Artificial Sequence
  Synthetic
ctgggctgggagcagcctc

SEQ ID NO: 265 - DNA, 23 bp, Artificial Sequence
  Synthetic
cactcgctggcctgtttcatgtc

SEQ ID NO: 266 - DNA, 22 bp, Artificial Sequence
  Synthetic
ctggaatccggtgtcgaagtgg

SEQ ID NO: 267 - DNA, 20 bp, Artificial Sequence
  Synthetic
ctcggcccctgcactgtttc

SEQ ID NO: 268 - DNA, 22 bp, Artificial Sequence
  Synthetic
gaggcaagaaggagtgtcaggg

SEQ ID NO: 269 - DNA, 23 bp, Artificial Sequence
  Synthetic
agtcctgtggtgaggtgacgagg

SEQ ID NO: 270 - DNA, 23 bp, Artificial Sequence
  Synthetic
gccaccatggtgtctttgctttc

SEQ ID NO: 271 - DNA, 22 bp, Artificial Sequence
  Synthetic
accggattccagctgggaaatg

SEQ ID NO: 272 - DNA, 21 bp, Artificial Sequence
  Synthetic
accgggcacctgtactcctca

SEQ ID NO: 273 - DNA, 22 bp, Artificial Sequence
  Synthetic
gcatgagctaaggcacccagac

SEQ ID NO: 274 - DNA, 27 bp, Artificial Sequence
  Synthetic
acggacgcggagttacagcacaggtgc

SEQ ID NO: 275 - DNA, 28 bp, Artificial Sequence
  Synthetic
cgcgccgaggcaggtagtcggtgagatc

SEQ ID NO: 276 - DNA, 22 bp, Artificial Sequence
  Synthetic
cccgcgccacccacactgagcc

SEQ ID NO: 277 - DNA, 32 bp, Artificial Sequence
  Synthetic
aagagtagccacgctcggtgaggatcttcatt

SEQ ID NO: 278 - DNA, 22 bp, Artificial Sequence
  Synthetic
gcagtggcagggggcctggtgt

SEQ ID NO: 279 - DNA, 27 bp, Artificial Sequence
  Synthetic
atgacgtggcagacggtagcgtgcagc

SEQ ID NO: 280 - DNA, 24 bp, Artificial Sequence
  Synthetic
cgcgccgaggagtagcgtgcagcc

SEQ ID NO: 281 - DNA, 41 bp, Artificial Sequence; identical to SEQ ID NO: 84
  Synthetic

SEQ ID NO: 282 - DNA, 41 bp, Artificial Sequence; identical to SEQ ID NO: 85
  Synthetic

SEQ ID NO: 283 - DNA, 22 bp, Artificial Sequence
  Synthetic
caggcggcctcctcggtcacct

SEQ ID NO: 284 - DNA, 24 bp, Artificial Sequence
  Synthetic
cgcgccgaggcactgctccagcga

SEQ ID NO: 285 - DNA, 27 bp, Artificial Sequence
  Synthetic
atgacgtggcagaccctgctccagcga

SEQ ID NO: 286 - DNA, 42 bp, Artificial Sequence; identical to SEQ ID NO: 74
  Synthetic

SEQ ID NO: 287 - DNA, 41 bp, Artificial Sequence; identical to SEQ ID NO: 75
  Synthetic

SEQ ID NO: 288 - DNA, 25 bp, Artificial Sequence
  Synthetic
ccttacccgcatctcccacccccat

SEQ ID NO: 289 - DNA, 24 bp, Artificial Sequence
  Synthetic
cgcgccgaggagacgcccctttcg

SEQ ID NO: 290 - DNA, 28 bp, Artificial Sequence
  Synthetic
atgacgtggcagacggacgcccctttcg

SEQ ID NO: 291 - DNA, 44 bp, Artificial Sequence; identical to SEQ ID NO: 69
  Synthetic

SEQ ID NO: 292 - DNA, 44 bp, Artificial Sequence; identical to SEQ ID NO: 70
  Synthetic

SEQ ID NO: 293 - DNA, 24 bp, Artificial Sequence
  Synthetic
gctgggctgggtcccaggtcatct

SEQ ID NO: 294 - DNA, 27 bp, Artificial Sequence
  Synthetic
cgcgccgaggctgtgctcagttagcag

SEQ ID NO: 295 - DNA, 30 bp, Artificial Sequence
  Synthetic
atgacgtggcagaccgtgctcagttagcag

SEQ ID NO: 296 - DNA, 47 bp, Artificial Sequence; identical to SEQ ID NO: 64
  Synthetic

SEQ ID NO: 297 - DNA, 46 bp, Artificial Sequence; identical to SEQ ID NO: 65
  Synthetic

SEQ ID NO: 298 - DNA, 30 bp, Artificial Sequence; identical to SEQ ID NO: 166
  Synthetic

SEQ ID NO: 299 - DNA, 33 bp, Artificial Sequence
  Synthetic
atgacgtggcagacgcaggttctcatcattgaa

SEQ ID NO: 300 - DNA, 30 bp, Artificial Sequence
  Synthetic
cgcgccgaggacaggttctcatcattgaag

SEQ ID NO: 301 - DNA, 55 bp, Artificial Sequence; identical to SEQ ID NO: 169
  Synthetic

SEQ ID NO: 302 - DNA, 55 bp, Artificial Sequence; identical to SEQ ID NO: 170
  Synthetic

SEQ ID NO: 303 - DNA, 31 bp, Artificial Sequence; identical to SEQ ID NO: 56
  Synthetic

SEQ ID NO: 304 - DNA, 25 bp, Artificial Sequence
  Synthetic
cgcgccgaggccccatccccctatg

SEQ ID NO: 305 - DNA, 29 bp, Artificial Sequence
  Synthetic
atgacgtggcagacgcccatccccctatg

SEQ ID NO: 306 - DNA, 51 bp, Artificial Sequence; identical to SEQ ID NO: 59
  Synthetic

SEQ ID NO: 307 - DNA, 51 bp, Artificial Sequence; identical to SEQ ID NO: 60
  Synthetic

SEQ ID NO: 308 - DNA, 21 bp, Artificial Sequence
  Synthetic
ccggggctgtccagtgggcat

SEQ ID NO: 309 - DNA, 23 bp, Artificial Sequence
  Synthetic
cgcgccgaggcagtgggcaccga

SEQ ID NO: 310 - DNA, 30 bp, Artificial Sequence
  Synthetic
atgacgtggcagacccgagaagctgaagtg

SEQ ID NO: 311 - DNA, 51 bp, Artificial Sequence; identical to SEQ ID NO: 14
  Synthetic

SEQ ID NO: 312 - DNA, 42 bp, Artificial Sequence; identical to SEQ ID NO: 15
  Synthetic

SEQ ID NO: 313 - DNA, 19 bp, Artificial Sequence
  Synthetic
cccgaagcggcgccgcaat

SEQ ID NO: 314 - DNA, 24 bp, Artificial Sequence
  Synthetic
cgcgccgaggctgcagagggaggg

SEQ ID NO: 315 - DNA, 28 bp, Artificial Sequence
  Synthetic
atgacgtggcagacgtgcagagggaggg

SEQ ID NO: 316 - DNA, 38 bp, Artificial Sequence; identical to SEQ ID NO: 139
  Synthetic

SEQ ID NO: 317 - DNA, 38 bp, Artificial Sequence; identical to SEQ ID NO: 140
  Synthetic

SEQ ID NO: 318 - DNA, 27 bp, Artificial Sequence
  Synthetic
ggctagaagcactgrtgcccctggcct

SEQ ID NO: 319 - DNA, 32 bp, Artificial Sequence
  Synthetic
atgacgtggcagacgtgatagtggccatcttc

SEQ ID NO: 320 - DNA, 28 bp, Artificial Sequence
  Synthetic
cgcgccgaggatgatagtggccatcttc

SEQ ID NO: 321 - DNA, 50 bp, Artificial Sequence
  Synthetic
gcaggaagatggccactatcacggccaggggcaycagtgcttctagcccc

SEQ ID NO: 322 - DNA, 50 bp, Artificial Sequence
  Synthetic
gcaggaagatggccactatcatggccaggggcaycagtgcttctagcccc

SEQ ID NO: 323 - DNA, 30 bp, Artificial Sequence
  Synthetic
agccttttggaagcgtaggaccttgccagt

SEQ ID NO: 324 - DNA, 28 bp, Artificial Sequence
  Synthetic
atgacgtggcagacccagcgctgggata

SEQ ID NO: 325 - DNA, 25 bp, Artificial Sequence
  Synthetic
cgcgccgaggacagcgctgggatat

SEQ ID NO: 326 - DNA, 50 bp, Artificial Sequence
  Synthetic
ctgcatatcccagcgctggctggcaaggtcctacgcttccaaaaggcttt

SEQ ID NO: 327 - DNA, 50 bp, Artificial Sequence
  Synthetic
ctgcatatcccagcgctgtctggcaaggtcctacgcttccaaaaggcttt

SEQ ID NO: 328 - DNA, 29 bp, Artificial Sequence
  Synthetic
ctgagctaggtccagcagcctgaggaaga

SEQ ID NO: 329 - DNA, 29 bp, Artificial Sequence
  Synthetic
atgacgtggcagaccgagggtcgtcgtac

SEQ ID NO: 330 - DNA, 25 bp, Artificial Sequence
  Synthetic
cgcgccgaggtgagggtcgtcgtac

SEQ ID NO: 331 - DNA, 48 bp, Artificial Sequence
  Synthetic
tcgagtacgacgaccctcgcttcctcaggctgctggacctagctcagg

SEQ ID NO: 332 - DNA, 48 bp, Artificial Sequence
  Synthetic
tcgagtacgacgaccctcacttcctcaggctgctggacctagctcagg

SEQ ID NO: 333 - DNA, 18 bp, Artificial Sequence
  Synthetic
cgggctacccatgggaca

SEQ ID NO: 334 - DNA, 31 bp, Artificial Sequence
  Synthetic
tctggtattaagccgtaatttgcatgattga

SEQ ID NO: 335 - DNA, 19 bp, Artificial Sequence
  Synthetic
ctgggctgggagcagcctc

SEQ ID NO: 336 - DNA, 23 bp, Artificial Sequence
  Synthetic
cactcgctggcctgtttcatgtc

SEQ ID NO: 337 - DNA, 22 bp, Artificial Sequence
  Synthetic
ctggaatccggtgtcgaagtgg

SEQ ID NO: 338 - DNA, 20 bp, Artificial Sequence
  Synthetic
ctcggcccctgcactgtttc

SEQ ID NO: 339 - DNA, 22 bp, Artificial Sequence
  Synthetic
gaggcaagaaggagtgtcaggg

SEQ ID NO: 340 - DNA, 23 bp, Artificial Sequence
  Synthetic
agtcctgtggtgaggtgacgagg

SEQ ID NO: 341 - DNA, 15 bp, Artificial Sequence
  Synthetic
ggtagtgaggcaggt

SEQ ID NO: 342 - DNA, 16 bp, Artificial Sequence
  Synthetic
gcttctggtaggggag

SEQ ID NO: 343 - DNA, 19 bp, Artificial Sequence
  Synthetic
aaataggactaggacctgt

SEQ ID NO: 344 - DNA, 15 bp, Artificial Sequence
  Synthetic
gggtcccacggaaat

SEQ ID NO: 345 - DNA, 12 bp, Artificial Sequence
  Synthetic
catggccacgcg

SEQ ID NO: 346 - DNA, 13 bp, Artificial Sequence
  Synthetic
ccggcacctctcg

SEQ ID NO: 347 - DNA, 14 bp, Artificial Sequence
  Synthetic
ccgtcctcctgcat

SEQ ID NO: 348 - DNA, 17 bp, Artificial Sequence
  Synthetic
cactctcaccttctcca

SEQ ID NO: 349 - DNA, 17 bp, Artificial Sequence
  Synthetic
gttctgtcccgagtatg

SEQ ID NO: 350 - DNA, 16 bp, Artificial Sequence
  Synthetic
tgcactgtttcccaga

SEQ ID NO: 351 - DNA, 17 bp, Artificial Sequence
  Synthetic
ctgacctcctccaacat

SEQ ID NO: 352 - DNA, 15 bp, Artificial Sequence
  Synthetic
gggctatcaccaggt

SEQ ID NO: 353 - DNA, 17 bp, Artificial Sequence
  Synthetic
ctgacctcctccaacat

SEQ ID NO: 354 - DNA, 15 bp, Artificial Sequence
  Synthetic
gggctatcaccaggt

SEQ ID NO: 355 - DNA, 18 bp, Artificial Sequence
  Synthetic
aaggctttgcaggcttca

SEQ ID NO: 356 - DNA, 19 bp, Artificial Sequence
  Synthetic
gctcggactacggtcatca

SEQ ID NO: 357 - DNA, 18 bp, Artificial Sequence
  Synthetic
tggaatccggtgtcgaag

SEQ ID NO: 358 - DNA, 20 bp, Artificial Sequence
  Synthetic
gaaatctctgacgtggatag

SEQ ID NO: 359 - DNA, 19 bp, Artificial Sequence
  Synthetic
gtacctcctatccacgtca

SEQ ID NO: 360 - DNA, 19 bp, Artificial Sequence
  Synthetic
cactccttcttgcctccta

SEQ ID NO: 361 - DNA, 19 bp, Artificial Sequence
  Synthetic
gcaagaaggagtgtcaggg

SEQ ID NO: 362 - DNA, 19 bp, Artificial Sequence
  Synthetic
ctgtggtgaggtgacgagg

SEQ ID NO: 363 - DNA, 31 bp, Artificial Sequence; identical to SEQ ID NO: 56
  Synthetic

SEQ ID NO: 364 - DNA, 26 bp, Artificial Sequence
  Synthetic
cgcgccgaggccccatccccctatgv

SEQ ID NO: 365 - DNA, 27 bp, Artificial Sequence
  Synthetic
acggacgcggaggcccatccccctatv

SEQ ID NO: 366 - DNA, 51 bp, Artificial Sequence; identical to SEQ ID NO: 59
  Synthetic

SEQ ID NO: 367 - DNA, 51 bp, Artificial Sequence; identical to SEQ ID NO: 60
  Synthetic

SEQ ID NO: 368 - DNA, 25 bp, Artificial Sequence
  Synthetic
ccttacccgcatctcccacccccat

SEQ ID NO: 369 - DNA, 25 bp, Artificial Sequence
  Synthetic
cgcgccgaggagacgcccctttcgv

SEQ ID NO: 370 - DNA, 26 bp, Artificial Sequence
  Synthetic
acggacgcggagggacgcccctttcv

SEQ ID NO: 371 - DNA, 44 bp, Artificial Sequence; identical to SEQ ID NO: 69
  Synthetic

SEQ ID NO: 372 - DNA, 44 bp, Artificial Sequence; identical to SEQ ID NO: 70
  Synthetic

SEQ ID NO: 373 - DNA, 23 bp, Artificial Sequence
  Synthetic
gggctcacgctgcacatccggac

SEQ ID NO: 374 - DNA, 29 bp, Artificial Sequence
  Synthetic
cgcgccgaggtgtaggatcatgagcaggv

SEQ ID NO: 375 - DNA, 30 bp, Artificial Sequence; identical to SEQ ID NO: 78
  Synthetic

SEQ ID NO: 376 - DNA, 46 bp, Artificial Sequence; identical to SEQ ID NO: 79
  Synthetic

SEQ ID NO: 377 - DNA, 46 bp, Artificial Sequence; identical to SEQ ID NO: 80
  Synthetic

SEQ ID NO: 378 - DNA, 22 bp, Artificial Sequence
  Synthetic
gcagtggcagggggcctggtgt

SEQ ID NO: 379 - DNA, 26 bp, Artificial Sequence
  Synthetic
acggacgcggagggtagcgtgcagcv

SEQ ID NO: 380 - DNA, 25 bp, Artificial Sequence
  Synthetic
cgcgccgaggagtagcgtgcagccv

SEQ ID NO: 381 - DNA, 41 bp, Artificial Sequence; identical to SEQ ID NO: 84
  Synthetic

SEQ ID NO: 382 - DNA, 41 bp, Artificial Sequence; identical to SEQ ID NO: 85
  Synthetic

SEQ ID NO: 383 - DNA, 23 bp, Artificial Sequence
  Synthetic
aggccctgaccctccctctgcat

SEQ ID NO: 384 - DNA, 22 bp, Artificial Sequence
  Synthetic
cgcgccgagggttgcggcgccv

SEQ ID NO: 385 - DNA, 24 bp, Artificial Sequence
  Synthetic
acggacgcggagcttgcggcgccv

SEQ ID NO: 386 - DNA, 39 bp, Artificial Sequence; identical to SEQ ID NO: 89
  Synthetic

SEQ ID NO: 387 - DNA, 39 bp, Artificial Sequence; identical to SEQ ID NO: 90
  Synthetic

SEQ ID NO: 388 - DNA, 23 bp, Artificial Sequence
  Synthetic
gcgagcagaggcgcttctccgtt

SEQ ID NO: 389 - DNA, 24 bp, Artificial Sequence
  Synthetic
cgcgccgagggtccaccttgcgcv

SEQ ID NO: 390 - DNA, 26 bp, Artificial Sequence
  Synthetic
acggacgcggagctccaccttgcgcv

SEQ ID NO: 391 - DNA, 41 bp, Artificial Sequence; identical to SEQ ID NO: 104
  Synthetic

SEQ ID NO: 392 - DNA, 41 bp, Artificial Sequence; identical to SEQ ID NO: 105
  Synthetic

SEQ ID NO: 393 - DNA, 27 bp, Artificial Sequence
  Synthetic
gcttcacaaagtggccctgggcatcct

SEQ ID NO: 394 - DNA, 26 bp, Artificial Sequence
  Synthetic
cgcgccgaggaggaagtgttcggggv

SEQ ID NO: 395 - DNA, 27 bp, Artificial Sequence
  Synthetic
acggacgcggaggggaagtgttcgggv

SEQ ID NO: 396 - DNA, 47 bp, Artificial Sequence; identical to SEQ ID NO: 174
  Synthetic

SEQ ID NO: 397 - DNA, 47 bp, Artificial Sequence; identical to SEQ ID NO: 175
  Synthetic

SEQ ID NO: 398 - DNA, 20 bp, Artificial Sequence
  Synthetic
cgcccgcctgtgcccatcaa

SEQ ID NO: 399 - DNA, 26 bp, Artificial Sequence
  Synthetic
cgcgccgaggcccagatcctgggttv

SEQ ID NO: 400 - DNA, 30 bp, Artificial Sequence; identical to SEQ ID NO: 208
  Synthetic

SEQ ID NO: 401 - DNA, 42 bp, Artificial Sequence; identical to SEQ ID NO: 209
  Synthetic

SEQ ID NO: 402 - DNA, 42 bp, Artificial Sequence; identical to SEQ ID NO: 210
  Synthetic

SEQ ID NO: 403 - DNA, 27 bp, Artificial Sequence
  Synthetic
caggctgctggacctagctcaggaggt

SEQ ID NO: 404 - DNA, 28 bp, Artificial Sequence
  Synthetic
acggacgcggagggatcgaaggaggagt

SEQ ID NO: 405 - DNA, 28 bp, Artificial Sequence
  Synthetic
cgcgccgagggactgaaggaggagtcgv

SEQ ID NO: 406 - DNA, 49 bp, Artificial Sequence
  Synthetic
cccgactcctccttcgatcccctcctgagctaggtccagcagcctgagt

SEQ ID NO: 407 - DNA, 49 bp, Artificial Sequence; identical to SEQ ID NO: 114
  Synthetic

SEQ ID NO: 408 - DNA, 30 bp, Artificial Sequence
  Synthetic
gccctacaccactgccgtgattcatgaggc

SEQ ID NO: 409 - DNA, 25 bp, Artificial Sequence
  Synthetic
cgcgccgaggtgtgcagcgctttgv

SEQ ID NO: 410 - DNA, 26 bp, Artificial Sequence
  Synthetic
acggacgcggagtgcagcgctttggv

SEQ ID NO: 411 - DNA, 51 bp, Artificial Sequence
  Synthetic
tgtccccaaagcgctgcacacctcatgaatcacggcagtggtgtagggcat

SEQ ID NO: 412 - DNA, 49 bp, Artificial Sequence
  Synthetic
tgtccccaaagcgctgcacctcatgaatcacggcagtggtgtagggcat

SEQ ID NO: 413 - DNA, 30 bp, Artificial Sequence
  Synthetic
catcrgtgctgaaggatgaggccgtctggt

SEQ ID NO: 414 - DNA, 27 bp, Artificial Sequence
  Synthetic
acggacgcggaggagaagcccttccgv

SEQ ID NO: 415 - DNA, 26 bp, Artificial Sequence
  Synthetic
cgcgccgaggaagaagcccttccgcv

SEQ ID NO: 416 - DNA, 50 bp, Artificial Sequence
  Synthetic
ggaagcggaagggcttctcccagacggcctcatccttcagcacygatgac

SEQ ID NO: 417 - DNA, 50 bp, Artificial Sequence
  Synthetic
ggaagcggaagggcttcttccagacggcctcatccttcagcacygatgac

SEQ ID NO: 418 - DNA, 19 bp, Artificial Sequence
  Synthetic
ggccccctgccactgccct

SEQ ID NO: 419 - DNA, 25 bp, Artificial Sequence
  Synthetic
acggacgcggaggggctgggcaacv

SEQ ID NO: 420 - DNA, 24 bp, Artificial Sequence
  Synthetic
cgcgccgaggaggctgggcaaccv

SEQ ID NO: 421 - DNA, 37 bp, Artificial Sequence
  Synthetic
agcaggttgcccagcccgggcagtggcagggggcctg

SEQ ID NO: 422 - DNA, 37 bp, Artificial Sequence
  Synthetic
agcaggttgcccagcctgggcagtggcagggggcctg

SEQ ID NO: 423 - DNA, 26 bp, Artificial Sequence
  Synthetic
cgccgcttcgagtacgacgaccctct

SEQ ID NO: 424 - DNA, 25 bp, Artificial Sequence
  Synthetic
cgcgccgagggcttcctcaggctgv

SEQ ID NO: 425 - DNA, 28 bp, Artificial Sequence
  Synthetic
acggacgcggagacttcctcaggctgcv

SEQ ID NO: 426 - DNA, 46 bp, Artificial Sequence
  Synthetic
tccagcagcctgaggaagcgagggtcgtcgtactcgaagcggcgcc

SEQ ID NO: 427 - DNA, 46 bp, Artificial Sequence
  Synthetic
tccagcagcctgaggaagtgagggtcgtcgtactcgaagcggcgcc

SEQ ID NO: 428 - DNA, 21 bp, Artificial Sequence
  Synthetic
ccctcccctccccacaggcct

SEQ ID NO: 429 - DNA, 23 bp, Artificial Sequence
  Synthetic
cgcgccgagggccgtgcatgccv

SEQ ID NO: 430 - DNA, 26 bp, Artificial Sequence
  Synthetic
acggacgcggagaccgtgcatgcctv

SEQ ID NO: 431 - DNA, 39 bp, Artificial Sequence
  Synthetic
cccgaggcatgcacggcggcctgtggggaggggaggggc

SEQ ID NO: 432 - DNA, 39 bp, Artificial Sequence
  Synthetic
cccgaggcatgcacggtggcctgtggggaggggaggggc

SEQ ID NO: 433 - DNA, 23 bp, Artificial Sequence
  Synthetic
agaagcactggtgcccctggcct

SEQ ID NO: 434 - DNA, 29 bp, Artificial Sequence
  Synthetic
cgcgccgagggtgatagtggccatcttcv

SEQ ID NO: 435 - DNA, 31 bp, Artificial Sequence
  Synthetic
acggacgcggagatgatagtggccatcttcv

SEQ ID NO: 436 - DNA, 46 bp, Artificial Sequence
  Synthetic
gcaggaagatggccactatcacggccaggggcaccagtgcttctag

SEQ ID NO: 437 - DNA, 46 bp, Artificial Sequence
  Synthetic
gcaggaagatggccactatcatggccaggggcaccagtgcttctag

SEQ ID NO: 438 - DNA, 28 bp, Artificial Sequence
  Synthetic
ggccgtgtccaacaggagatcgacgact

SEQ ID NO: 439 - DNA, 28 bp, Artificial Sequence
  Synthetic
acggacgcggaggtgatagggcaggtgv

SEQ ID NO: 440 - DNA, 27 bp, Artificial Sequence
  Synthetic
cgcgccgaggatgatagggcaggtgcv

SEQ ID NO: 441 - DNA, 49 bp, Artificial Sequence
  Synthetic
cgccgcacctgccctatcacgtcgtcgatctcctgttggacacggcctg

SEQ ID NO: 442 - DNA, 49 bp, Artificial Sequence
  Synthetic
cgccgcacctgccctatcatgtcgtcgatctcctgttggacacggcctg

SEQ ID NO: 443 - DNA, 25 bp, Artificial Sequence
  Synthetic
tggccactatcayggccaggggcaa

SEQ ID NO: 444 - DNA, 28 bp, Artificial Sequence
  Synthetic
acggacgcggagccagtgcttctagccv

SEQ ID NO: 445 - DNA, 27 bp, Artificial Sequence
  Synthetic
cgcgccgaggtcagtgcttctagcccv

SEQ ID NO: 446 - DNA, 46 bp, Artificial Sequence
  Synthetic
tatggggctagaagcactggtgcccctggccrtgatagtggccatc

SEQ ID NO: 447 - DNA, 46 bp, Artificial Sequence
  Synthetic
tatggggctagaagcactgatgcccctggccrtgatagtggccatc

SEQ ID NO: 448 - DNA, 24 bp, Artificial Sequence
  Synthetic
gctgggctgggtcccaggtcatct

SEQ ID NO: 449 - DNA, 30 bp, Artificial Sequence
  Synthetic
acggacgcggagctgtgctcagttagcagv

SEQ ID NO: 450 - DNA, 27 bp, Artificial Sequence
  Synthetic
cgcgccgaggcgtgctcagttagcagv

SEQ ID NO: 451 - DNA, 47 bp, Artificial Sequence; identical to SEQ ID NO: 64
  Synthetic

SEQ ID NO: 452 - DNA, 46 bp, Artificial Sequence; identical to SEQ ID NO: 65
  Synthetic

SEQ ID NO: 453 - DNA, 22 bp, Artificial Sequence
  Synthetic
caggcggcctcctcggtcacct

SEQ ID NO: 454 - DNA, 25 bp, Artificial Sequence
  Synthetic
cgcgccgaggcactgctccagcgav

SEQ ID NO: 455 - DNA, 27 bp, Artificial Sequence
  Synthetic
acggacgcggagcctgctccagcgacv

SEQ ID NO: 456 - DNA, 42 bp, Artificial Sequence; identical to SEQ ID NO: 74
  Synthetic

SEQ ID NO: 457 - DNA, 41 bp, Artificial Sequence; identical to SEQ ID NO: 75
  Synthetic

SEQ ID NO: 458 - DNA, 22 bp, Artificial Sequence
  Synthetic
cttgccttgggaacgcggccct

SEQ ID NO: 459 - DNA, 28 bp, Artificial Sequence
  Synthetic
cgcgccgagggaaacccaggatctgggv

SEQ ID NO: 460 - DNA, 30 bp, Artificial Sequence
  Synthetic
acggacgcggagaaaacccaggatctgggv

SEQ ID NO: 461 - DNA, 43 bp, Artificial Sequence; identical to SEQ ID NO: 149
  Synthetic

SEQ ID NO: 462 - DNA, 43 bp, Artificial Sequence; identical to SEQ ID NO: 150
  Synthetic

SEQ ID NO: 463 - DNA, 30 bp, Artificial Sequence; identical to SEQ ID NO: 166
  Synthetic

SEQ ID NO: 464 - DNA, 33 bp, Artificial Sequence
  Synthetic
acggacgcggaggcaggttctcatcattgaagv

SEQ ID NO: 465 - DNA, 32 bp, Artificial Sequence
  Synthetic
cgcgccgaggacaggttctcatcattgaagcv

SEQ ID NO: 466 - DNA, 55 bp, Artificial Sequence; identical to SEQ ID NO: 169
  Synthetic

SEQ ID NO: 467 - DNA, 55 bp, Artificial Sequence; identical to SEQ ID NO: 170
  Synthetic

SEQ ID NO: 468 - DNA, 25 bp, Artificial Sequence
  Synthetic
tgtgccgccttcgccraccactcyc

SEQ ID NO: 469 - DNA, 25 bp, Artificial Sequence
  Synthetic
cgcgccgaggggtgggtgatgggcv

SEQ ID NO: 470 - DNA, 27 bp, Artificial Sequence
  Synthetic
acggacgcggagtgtgggtgatgggcv

SEQ ID NO: 471 - DNA, 44 bp, Artificial Sequence
  Synthetic
ttctgcccatcacccaccrgagtggtyggcgaaggcggcacaaa

SEQ ID NO: 472 - DNA, 44 bp, Artificial Sequence
  Synthetic
ttctgcccatcacccacargagtggtyggcgaaggcggcacaaa

SEQ ID NO: 473 - DNA, 25 bp, Artificial Sequence
  Synthetic
tgtgccgccttcgccraccactcyt

SEQ ID NO: 474 - DNA, 25 bp, Artificial Sequence
  Synthetic
cgcgccgaggggtgggtgatgggcv

SEQ ID NO: 475 - DNA, 27 bp, Artificial Sequence
  Synthetic
acggacgcggagagtgggtgatgggcv

SEQ ID NO: 476 - DNA, 44 bp, Artificial Sequence; identical to SEQ ID NO: 471
  Synthetic

SEQ ID NO: 477 - DNA, 44 bp, Artificial Sequence
  Synthetic
ttctgcccatcacccactrgagtggtyggcgaaggcggcacaaa

SEQ ID NO: 478 - DNA, 22 bp, Artificial Sequence
  Synthetic
tgcgcgaggcgmtggtgaccct

SEQ ID NO: 479 - DNA, 25 bp, Artificial Sequence
  Synthetic
cgcgccgaggacggcgaggacaccv

SEQ ID NO: 480 - DNA, 26 bp, Artificial Sequence
  Synthetic
acggacgcggaggcggcgaggacacv

SEQ ID NO: 481 - DNA, 42 bp, Artificial Sequence
  Synthetic
tcggcsgtgtcctcgccgtgggtcaccakcgcctcgcgcacg

SEQ ID NO: 482 - DNA, 42 bp, Artificial Sequence
  Synthetic
tcggcsgtgtcctcgccgcgggtcaccakcgcctcgcgcacg

SEQ ID NO: 483 - DNA, 25 bp, Artificial Sequence
  Synthetic
gctgggtcccaggtcatccgtgctt

SEQ ID NO: 484 - DNA, 26 bp, Artificial Sequence
  Synthetic
gctgggtcccaggtcatcctgtgctt

SEQ ID NO: 485 - DNA, 25 bp, Artificial Sequence
  Synthetic
cgcgccgaggcagcagctcatccag

SEQ ID NO: 486 - DNA, 29 bp, Artificial Sequence
  Synthetic
acggacgcggagcagttagcagctcatcc

SEQ ID NO: 487 - DNA, 47 bp, Artificial Sequence
  Synthetic
acccagctggatgagctgctgagcacggatgacctgggacccagccc

SEQ ID NO: 488 - DNA, 48 bp, Artificial Sequence
  Synthetic
acccagctggatgagctgctgagcacaggatgacctgggacccagccc

SEQ ID NO: 489 - DNA, 51 bp, Artificial Sequence
  Synthetic
acccagctggatgagctgctaactgagcacggatgacctgggacccagccc

SEQ ID NO: 490 - DNA, 52 bp, Artificial Sequence
  Synthetic
acccagctggatgagctgctaactgagcacaggatgacctgggacccagccc

SEQ ID NO: 491 - DNA, 24 bp, Artificial Sequence
  Synthetic
ccaccgtggcagccactctcaccc

SEQ ID NO: 492 - DNA, 27 bp, Artificial Sequence
  Synthetic
cgcgccgaggttctccatctctgccav

SEQ ID NO: 493 - DNA, 28 bp, Artificial Sequence
  Synthetic
acggacgcggagtccatctctgccaggv

SEQ ID NO: 494 - DNA, 48 bp, Artificial Sequence; identical to SEQ ID NO: 164
  Synthetic

SEQ ID NO: 495 - DNA, 45 bp, Artificial Sequence; identical to SEQ ID NO: 165
  Synthetic

SEQ ID NO: 496 - DNA, 23 bp, Artificial Sequence
  Synthetic
ccccargacgcccctttcgccct

SEQ ID NO: 497 - DNA, 26 bp, Artificial Sequence
  Synthetic
cgcgccgaggctttcgcccctttcgv

SEQ ID NO: 498 - DNA, 29 bp, Artificial Sequence
  Synthetic
acggacgcggagcaacggtctcttggacv

SEQ ID NO: 499 - DNA, 62 bp, Artificial Sequence
  Synthetic
ctttgtccaagagaccgttggggcgaaaggggcgaaaggggcgaaaggggcgtcytgggggt

SEQ ID NO: 500 - DNA, 44 bp, Artificial Sequence
  Synthetic
ctttgtccaagagaccgttggggcgaaaggggcgtcytgggggt

SEQ ID NO: 501 - DNA, 24 bp, Artificial Sequence
  Synthetic
ggagggcggcagaggtsctgaggt

SEQ ID NO: 502 - DNA, 30 bp, Artificial Sequence
  Synthetic
acggacgcggagctcccctaccagaagcav

SEQ ID NO: 503 - DNA, 25 bp, Artificial Sequence
  Synthetic
cgcgccgaggatgccccaccagaav

SEQ ID NO: 504 - DNA, 47 bp, Artificial Sequence
  Synthetic
atgtttgcttctggtaggggagcctcagsacctctgccgccctccag

SEQ ID NO: 505 - DNA, 47 bp, Artificial Sequence
  Synthetic
atgtttgcttctggtggggcatcctcagsacctctgccgccctccag

SEQ ID NO: 506 - DNA, 32 bp, Artificial Sequence
  Synthetic
cccaccatccatgtttgcttctggtrgggsac

SEQ ID NO: 507 - DNA, 26 bp, Artificial Sequence
  Synthetic
cgcgccgagggcctcagcacctctgv

SEQ ID NO: 508 - DNA, 29 bp, Artificial Sequence
  Synthetic
acggacgcggagtcctcaggacctctgcv

SEQ ID NO: 509 - DNA, 53 bp, Artificial Sequence
  Synthetic
ggcggcagaggtgctgaggctscccyaccagaagcaaacatggatggtgggtg

SEQ ID NO: 510 - DNA, 53 bp, Artificial Sequence
  Synthetic
ggcggcagaggtcctgaggatscccyaccagaagcaaacatggatggtgggtg

SEQ ID NO: 511 - DNA, 26 bp, Artificial Sequence
  Synthetic
ggagggcggcagaggtsctgaggmtt

SEQ ID NO: 512 - DNA, 31 bp, Artificial Sequence
  Synthetic
acggacgcggagcccctaccagaagcaaacv

SEQ ID NO: 513 - DNA, 25 bp, Artificial Sequence
  Synthetic
cgcgccgagggccccaccagaagcv

SEQ ID NO: 514 - DNA, 47 bp, Artificial Sequence
  Synthetic
atgtttgcttctggtaggggagcctcagcacctctgccgccctccag

SEQ ID NO: 515 - DNA, 47 bp, Artificial Sequence
  Synthetic
atgtttgcttctggtggggcaacctcaggacctctgccgccctccag

SEQ ID NO: 516 - DNA, 26 bp, Artificial Sequence
  Synthetic
ccacccatgtttgctggtggtggggt

SEQ ID NO: 517 - DNA, 31 bp, Artificial Sequence
  Synthetic
acccaccatccatgtttgcttctggtrgggt

SEQ ID NO: 518 - DNA, 28 bp, Artificial Sequence
  Synthetic
acggacgcggaggagcctcagcacctcv

SEQ ID NO: 519 - DNA, 28 bp, Artificial Sequence
  Synthetic
cgcgccgaggcatcctcaggacctctgv

SEQ ID NO: 520 - DNA, 54 bp, Artificial Sequence
  Synthetic
ggcggcagaggtgctgaggctcccctaccagaagcaaacatggatggtgggtga

SEQ ID NO: 521 - DNA, 54 bp, Artificial Sequence
  Synthetic
ggcggcagaggtcctgaggatgccccaccagaagcaaacatggatggtgggtga

SEQ ID NO: 522 - DNA, 22 bp, Artificial Sequence
  Synthetic
cccgcgccacccacactgagcc

SEQ ID NO: 523 - DNA, 32 bp, Artificial Sequence; identical to SEQ ID NO: 257
  Synthetic

SEQ ID NO: 524 - DNA, 27 bp, Artificial Sequence
  Synthetic
acggacgcggagttacagcacaggtgc

SEQ ID NO: 525 - DNA, 28 bp, Artificial Sequence
  Synthetic
cgcgccgaggcaggtagtcggtgagatc

SEQ ID NO: 526 - DNA, 43 bp, Artificial Sequence
  Synthetic
ggaccgcacctgtgctgtaagctcagtgtgggtggcgcggggc

SEQ ID NO: 527 - DNA, 55 bp, Artificial Sequence
  Synthetic
cgcgatctcaccgactacctgaatgaagatcctcaccgagcgtggctactccttc

SEQ ID NO: 528 - DNA, 33 bp, Artificial Sequence
  Synthetic
tcttcggccttttggccgagagacctcggcgcg

SEQ ID NO: 529 - DNA, 35 bp, Artificial Sequence
  Synthetic
tcttcggccttttggccgagagactccgcgtccgt

(19) United States
(12) Patent Application Publication
(10) Pub. No.: US 2005/0196771 A1
(43) Pub. Date: Sep. 8, 2005

(54) Characterization of CYP 2D6 genotypes

(76) Inventors: Matt Neville, Oxford (GB); Monika de Arruda Indig, Madison, WI (US); Feng Cao, Hartland, WI (US); Mary C. Oldenburg, Madison, WI (US); James A. Koelbl, Cross Plains, WI (US); Brian D. Aizenstein, Madison, WI (US); Keith Davey, Middleton, WI (US)

Correspondence Address: Mary Ann D. Brow;MEDLEN & CARROLL, LLP, Suite 350, 101 Howard Street, San Francisco, CA, 94105, US

(21) Appl. No.: 10/956,507

(22) Filed: Oct. 1, 2004

Related U.S. Application Data
(63) Continuation-in-part of application No. 10/617,070, filed on Jul. 10, 2003.
Application No. 10/617,070 is a continuation-in-part of application No. 10/411,954, filed on Apr. 11, 2003.
(60) Provisional application No. 60/508,220, filed on Oct. 2, 2003.
(60) Provisional application No. 60/371,819, filed on Apr. 11, 2002.

(51) Int. Cl. C12Q 1/68; G06F 19/00; G01N 33/48; G01N 33/50

(52) U.S. Cl. 435/6; 702/20

(57) ABSTRACT

The present invention provides methods and routines for developing and optimizing nucleic acid detection assays for use in basic research, clinical research, and for the development of clinical detection assays. The present invention also provides cytochrome p450 genotyping methods and reagents for genotyping assays and a genomic DNA copy number assays.

The present application claims priority to Provisional Application Ser. No. 60/508/220, filed Oct. 2, 2003, which is incorporated herein by reference in its entirety. The present application is a continuation-in-part of U.S. patent application Ser. No. 10/617,070, filed Jul. 10, 2003, which is a continuation-in-part of U.S. patent application Ser. No. 10/411,954, filed Apr. 11, 2003, which claims priority to Provisional Application Ser. No. 60/371,819, filed Apr. 11, 2002, each of which is incorporated herein by reference in their entireties.

### FIELD OF THE INVENTION

The present invention provides methods and routines for developing and optimizing nucleic acid detection assays for use in basic research, clinical research, and for the development of clinical detection assays. In particular, the present invention provides methods for characterizing cytochrome p450 (CYP) genes and alleles.

### BACKGROUND

As the Human Genome Project nears completion and the volume of genetic sequence information available increases, genomics research and subsequent drug design efforts increase as well. A number of institutions are actively mining the available genetic sequence information to identify correlations between genes, gene expression and phenotypes (e.g., disease states, metabolic responses, and the like). These analyses include an attempt to characterize the effect of gene mutations and genetic and gene expression heterogeneity in individuals and populations. However, despite the wealth of sequence information available, information on the frequency and clinical relevance of many polymorphisms and other variations has yet to be obtained and validated. For example, the human reference sequences used in current genome sequencing efforts do not represent an exact match for any one person's genome. In the Human Genome Project (HGP), researchers collected blood (female) or sperm (male) samples from a large number of donors. However, only a few samples were processed as DNA resources, and the source names are protected so neither donors nor scientists know whose DNA is being sequenced. The human genome sequence generated by the private genomics company Celera was based on DNA samples collected from five donors who identified themselves as Hispanic, Asian, Caucasian, or African-American. The small number of human samples used to generate the reference sequences does not reflect the genetic diversity among population groups and individuals. Attempts to analyze individuals based on the genome sequence information will often fail. For example, many genetic detection assays are based on the hybridization of probe oligonucleotides to a target region on genomic DNA or mRNA. Probes generated based on the reference sequences will often fail (e.g., fail to hybridize properly, fail to properly characterize the sequence at specific position of the target) because the target sequence for many individuals differs from the reference sequence. Differences may be on an individual-by-individual basis, but many follow regional population patterns (e.g., many correlate highly to race, ethnicity, geographic local, age, environmental exposure, etc.). With the limited utility of information currently available, the art is in need of systems and methods for acquiring, analyzing, storing, and applying large volumes of genetic information with the goal of providing an array of detection assay technologies for research and clinical analysis of biological samples.

The cytochrome p450 (CYP) superfamily comprises a group of enzymes that play an essential role in the biotransformation of medically relevant compounds. Accurate genotyping of members of this protein family is drawing increasing interest because allelic variants may result in either loss of efficacy or toxic accumulation. Debrisoquine 4-hydroxylase, or CYP2D6, is among the most widely studied of the cytochrome p450s. However, the complex genetics of this enzyme, encompassing its entire genomic region, offers numerous challenges to a genotyping strategy, such as pseudogenes, gene deletions and gene duplications. With this complexity, the art is in needs of systems and methods of characterizing (e.g., quantifying and genotyping) CYP genes and alleles.

### SUMMARY OF THE INVENTION

The present invention provides methods for characterizing CYP genes and alleles.

The present invention provides methods and routines for developing and optimizing nucleic acid detection assays for use in basic research, clinical research, and for the development of clinical detection assays.

In some embodiments, the present invention provides methods comprising; a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises a forward and a reverse primer sequence for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In other embodiments, the present invention provides methods comprising; a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises a forward and a reverse primer sequence for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set.

In particular embodiments, a method comprising; a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises; i) a forward primer sequence identical to at least a portion of the 5′ region for each of the Y target sequences, and ii) a reverse primer sequence identical to at least a portion of a complementary sequence of the 3′ region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In other embodiments, the present invention provides methods comprising a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises; i) a forward primer sequence identical to at least a portion of the 5′ region for each of the Y target sequences, and ii) a reverse primer sequence identical to at least a portion of a complementary sequence of the 3′ region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set.

In particular embodiments, the present invention provides methods comprising a) providing target sequence information for at least Y target sequences, wherein each of the target sequences comprises a single nucleotide polymorphism, b) determining where on each of the target sequences one or more assay probes would hybridize in order to detect the single nucleotide polymorphism such that a footprint region is located on each of the target sequences, and c) processing the target sequence information such that a primer set is generated, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In some embodiments, the present invention provides methods comprising a) providing target sequence information for at least Y target sequences, wherein each of the target sequences comprises a single nucleotide polymorphism, b) determining where on each of the target sequences one or more assay probes would hybridize in order to detect the single nucleotide polymorphism such that a footprint region is located on each of the target sequences, and c) processing the target sequence information such that a primer set is generated, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide T or G, and N[2]-N[1]-3′ of each of the forward and reverse primers is not complementary to N[2]-N[1]-3′ of any of the forward and reverse primers in the primer set.

In certain embodiments, the primer set is configured for performing a multiplex PCR reaction that amplifies at least Y amplicons, wherein each of the amplicons is defined by the position of the forward and reverse primers. In other embodiments, the primer set is generated as digital or printed sequence information. In some embodiments, the primer set is generated as physical primer oligonucleotides.

In certain embodiments, N[3]-N[2]-N[1]-3′ of each of the forward and reverse primers is not complementary to N[3]-N[2]-N[1]-3′ of any of the forward and reverse primers in the primer set. In other embodiments, the processing comprises initially selecting N[1] for each of the forward primers as the most 3′ A or C in the 5′ region. In certain embodiments, the processing comprises initially selecting N[1] for each of the forward primers as the most 3′ G or T in the 5′ region. In some embodiments, the processing comprises initially selecting N[1] for each of the forward primers as the most 3′ A or C in the 5′ region, and wherein the processing further comprises changing the N[1] to the next most 3′ A or C in the 5′ region for the forward primer sequences that fail the requirement that each of the forward primer's N[2]-N[1]-3′ is not complementary to N[2]-N[1]-3′ of any of the forward and reverse primers in the primer set.

In other embodiments, the processing comprises initially selecting N[1] for each of the reverse primers as the most 3′ A or C in the complement of the 3′ region. In some embodiments, the processing comprises initially selecting N[1] for each of the reverse primers as the most 3′ G or T in the complement of the 3′ region. In further embodiments, the processing comprises initially selecting N[1] for each of the reverse primers as the most 3′ A or C in the 3′ region, and wherein the processing further comprises changing the N[1] to the next most 3′ A or C in the 3′ region for the reverse primer sequences that fail the requirement that each of the reverse primer's N[2]-N[1]-3′ is not complementary to N[2]-N[1]-3′ of any of the forward and reverse primers in the primer set.

In particular embodiments, the footprint region comprises a single nucleotide polymorphism. In some embodiments, the footprint comprises a mutation. In some embodiments, the footprint region for each of the target sequences comprises a portion of the target sequence that hybridizes to one or more assay probes configured to detect the single nucleotide polymorphism. In certain embodiments, the footprint is this region where the probes hybridize. In other embodiments, the footprint further includes additional nucleotides on either end.

In some embodiments, the processing further comprises selecting N[5]-N[4]-N[3]-N[2]-N[1]-3′ for each of the forward and reverse primers such that less than 80 percent homology with a assay component sequence is present. In preferred embodiments, the assay component is a FRET probe sequence. In certain embodiments, the target sequence is about 300-500 base pairs in length, or about 200-600 base pair in length. In certain embodiments, Y is an integer between 2 and 500, or between 2-10,000.

In certain embodiments, the processing comprises selecting x for each of the forward and reverse primers such that each of the forward and reverse primers has a melting temperature with respect to the target sequence of approximately 50 degrees Celsius (e.g. 50 degrees, Celsius, or at least 50 degrees Celsius, and no more than 55 degrees Celsius). In preferred embodiments, the melting temperature of a primer (when hybridized to the target sequence) is at least 50 degrees Celsius, but at least 10 degrees different than a selected detection assay's optimal reaction temperature.

In some embodiments, the forward and reverse primer pair optimized concentrations are determined for the primer set. In other embodiments, the processing is automated. In further embodiments, the processing is automated with a processor.

In other embodiments, the present invention provides a kit comprising the primer set generated by the methods of the present invention, and at least one other component. (e.g. cleavage agent, polymerase, INVADER oligonucleotide). In certain embodiments, the present invention provides compositions comprising the primers and primer sets generated by the methods of the present invention.

In particular embodiments, the present invention provides methods comprising; a) providing; i) a user interface configured to receive sequence data, ii) a computer system having stored therein a multiplex PCR primer software application, and b) transmitting the sequence data from the user interface to the computer system, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, and c) processing the target sequence information with the multiplex PCR primer pair software application to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In some embodiments, the present invention provides methods comprising; a) providing; i) a user interface configured to receive sequence data, ii) a computer system having stored therein a multiplex PCR primer software application, and b) transmitting the sequence data from the user interface to the computer system, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, and c) processing the target sequence information with the multiplex PCR primer pair software application to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set.

In certain embodiments, the present invention provides systems comprising; a) a computer system configured to receive data from a user interface, wherein the user interface is configured to receive sequence data, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, b) a multiplex PCR primer pair software application operably linked to the user interface, wherein the multiplex PCR primer software application is configured to process the target sequence information to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set, and c) a computer system having stored therein the multiplex PCR primer pair software application, wherein the computer system comprises computer memory and a computer processor.

In other embodiments, the present invention provides systems comprising; a) a computer system configured to receive data from a user interface, wherein the user interface is configured to receive sequence data, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, b) a multiplex PCR primer pair software application operably linked to the user interface, wherein the multiplex PCR primer software application is configured to process the target sequence information to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set, and c) a computer system having stored therein the multiplex PCR primer pair software application, wherein the computer system comprises computer memory and a computer processor. In certain embodiments, the computer system is configured to return the primer set to the user interface.

In some embodiments, the present invention provides a comprehensive CYP2D6 genotyping strategy that combines an genotyping assay system (e.g., an INVADER assay system) and a genomic DNA copy number assay (e.g., with or with amplification of the target sequence). In other embodiments, the present invention provides a comprehensive CYP2D6 genotyping strategy that combines a PCR-genotyping assay system and a genomic DNA copy number assay.

In some embodiments, the method of characterizing a cytochrome p450 allele comprises providing a sample comprising at least Y target sequences, wherein each of said target sequences comprises at least a portion of a cytochrome p450 allele, and wherein each of said target sequences comprises a footprint region, a 5′ region immediately upstream of said footprint region, and a 3′ region immediately downstream of said footprint region, a primer set comprising a forward and a reverse primer sequence for each of said at least Y target sequences, at least one assay probe configured to detect a footprint region, wherein said primer set is configured for performing a multiplex PCR reaction that amplifies at least Y amplicons, wherein each of said amplicons is defined by the position of said forward and reverse primers, amplifying said Y target sequences with said primer set; and detecting at least one of said footprint regions with said assay probe. In some embodiments, said at least one footprint region of said Y target sequences comprises a polymorphism.

In other embodiments, the present invention provides methods for detecting at least one cytochrome p450 allele, comprising providing a sample comprising at least one cytochrome p450 allele, oligonucleotides configured to hybridize to said cytochrome p450 allele to form an invasive cleavage structure; and an agent that detects the presence of an invasive cleavage structure; and further comprises exposing said sample to said oligonucleotides and said agent. In preferred embodiments, said at least one cytochrome p450 allele comprises a CYP2D6 allele. In some particularly preferred embodiments, said exposing said sample to said oligonucleotides and said agent comprises exposing said sample to said oligonucleotides and said agent under conditions wherein an invasive cleavage structure is formed between said at least one cytochrome p450 allele and said oligonucleotides. In still other preferred embodiments, the method comprises detecting said invasive cleavage structure.

In some embodiments, the present invention provides a method of detecting the presence or copy number of a mutant allele in the presence of one or more pseudogenes sharing a related sequence with the wild type allele of the same gene. In some embodiments, the quantity of a mutant allele present in a sample is compared to the quantity of an invariant gene present in the sample, where said invariant gene is used as a reference in lieu of said wild type allele against which the quantity of any mutant allele is measured.

The present invention provides kits comprising an oligonucleotide detection assay configured for detecting at least one cytochrome p450 allele, wherein said kit comprises at least two oligonucleotides, and wherein two of said at least two oligonucleotides hybridize to both wild type and mutant cytochrome p450 alleles. In some preferred embodiments, said at least one cytochrome p450 allele a CYP2D6 allele. In some embodiments, said oligonucleotide detection assays comprise first and second oligonucleotides configured to form an invasive cleavage structure in combination with target sequences comprising said cytochrome p450 alleles. In preferred embodiments, said first oligonucleotide comprises a 5′ portion and a 3′ portion, wherein said 3′ portion is configured to hybridize to said target sequence, and wherein said 5′ portion is configured to not hybridize to said target sequence.

In some embodiments of the kits of the present invention, said oligonucleotide detection assays are selected from sequencing assays, polymerase chain reaction assays, hybridization assays, hybridization assays employing a probe complementary to a mutation, microarray assays, bead array assays, primer extension assays, enzyme mismatch cleavage assays, branched hybridization assays, rolling circle replication assays, NASBA assays, molecular beacon assays, cycling probe assays, ligase chain reaction assays, invasive cleavage structure assays, ARMS assays, and sandwich hybridization assays.

The present invention also provides kits comprising an oligonucleotide detection assay configured for detecting the number of CYP2D6 gene copies present in a sample and configured to identify the presence or absence of at least one (e.g., at least two or more) CYP2D6 associated polymorphisms. In some embodiments, the detection assay is configured to detect the copy of number of the CYP2D6 gene and, separately, the copy number of a least one portion of the CYP2D6 gene (e.g., to identify the copy number of a polymorphism associated with a duplication of only a portion of the CYP2D6 gene or genic region—for example, 31G>A, 100C>T, and 4180 G>C). In some embodiments, the CYP2D6 associated polymorphisms are selected from the group consisting of 19G>A, 31G>A, 100C>T, 124G>A, 221C>A, 833G>C, 984A>G, 1023C>T, 1039C>T, 1661G>C, 1707T>del, 1758G>A, 1758G>T, 1846G>A, 1863ins[TTTCGCCCC]2, 1943G>A, 1973insG, 2539-2542delAACT, 2549A>del, 2613-2615delAGA, 2850C>T, 2935A>C, 3183G>A, 3259insGT, 3853G>A, 3887T>C, 4042G>A, 4180G>C, gene copy number, copy number 31G, copy number 100T, and copy number 4180G. These polymorphisms, individually, are known in the art.

In some embodiments, the kit further comprises a control reagent for assessing CYP2D6 copy number. In some preferred embodiments, that control reagent comprises reagents (e.g., detection assay components) for detection of alpha-actin. In some embodiments, the control reagent comprises synthetic target nucleic acids having 0, 1, 2, 3, and/or 4 copies of a CYP2D6 gene sequence. In some embodiments, the control reagent comprises synthetic target nucleic acids having 0, 1, 2, 3, or 4 copies of a mutant CYP2D6 sequence.

The present invention further provides methods for detecting a CYP2D6 genotype of a sample, comprising: a) providing a sample comprising a target nucleic acid; and a detection assay configured to detect at least two CYP2D6 polymorphic sequences and to detect CYP2D6 copy number; and b) exposing said sample to said detection assay under conditions such that said at least two CYP2D6 polymorphic sequences are detected and CYP2D6 copy number is detected, thereby detecting a CYP2D6 genotype of said sample. In some embodiments, the target nucleic acid is amplified prior to said exposure step. In some embodiments, the detection assay is configured to detect the copy of number of the CYP2D6 gene and, separately, the copy number of a least one portion of the CYP2D6 gene. In some embodiments, the detection assay further detects a copy number of at least one of said polymorphic sequences.

The present invention also provides a method for genotyping a subject having a CYP2D6 gene (including information pertaining to the CYP2D6 associated genic sequences) comprising the steps of: a) detecting one or more (e.g., 2 or more, 5 or more, 25 or more, etc.) single nucleotide polymorphisms associated with the CYP2D6 gene in said subject; b) detecting the CYP2D6 gene copy number; c) optionally, if multi-copy number polymorphisms are present, detecting the copy number of the multi-copy number polymorphism; d) generating a genotype profile based on the information derived from steps a-c; and, in some embodiments, comparing said genotype profile to a predetermined CYP2D6 information matrix, such that a CYP2D6 genotype of said subject is determined. In some embodiments, the single nucleotide polymorphisms and the information matrix are selected (e.g., by including a sufficient number of polymorphisms in conjunction with the sufficient copy number information) such that over 99% of Caucasian ultra metabolizers and over 95% of intermediate and low metabolizer are genotyped for CYP2D6. In some embodiments, the predetermined CYP2D6 information matrix is stored in a computer memory. In some preferred embodiments, the method further comprises the step of using said CYP2D6 genotype in selecting a therapy for a subject (e.g., selecting an appropriate drug, selecting an appropriate dose of drug, avoiding certain drugs, etc.). In some embodiments, the method further comprises the step of comparing said CYP2D6 genotype to a drug interaction observed in said subject.

### DESCRIPTION OF THE FIGURES

The following figures form part of the present specification and are included to further demonstrate certain aspects and embodiments of the present invention. The invention may be better understood by reference to one or more of these figures in combination with the description of specific embodiments presented herein.

FIG. 1 shows a schematic diagram of SNP detection using the INVADER assay in a biplex format that includes INVADER oligonucleotides, probe oligonucleotides and FRET cassettes for detecting a two different alleles (e.g., differing by a single nucleotide) in a single reaction.

FIG. 2 shows an input target sequence and the result of processing this sequence with systems and routines of the present invention.

FIG. 3 shows an example of a basic work flow for highly multiplexed PCR using the INVADER Medically Associated Panel.

FIG. 4 shows a flow chart outlining the steps that may be performed in order to generated a primer set useful in multiplex PCR.

FIG. 5 shows some examples of PCR primers useful for amplifying various regions of CYP2D6.

FIG. 6 shows a schematic representation of the CYP2D6 genomic region and one embodiment of a triplex PCR strategy. A. The position of CYP2D6 in relation to its 2 pseudogenes CYP2D7 and CYP2D8. B. The positions of polymorphisms found within or bordering the nine CYP2D6 exons. The relative frequency of the different polymorphisms is indicated by the length of the arrow. Solid arrows indicate non-synonymous polymorphisms and hashed arrows synonymous. The position and base change of each polymorhpism is indicated at the end if the arrow. The asterisk (*) below the arrows indicates 11 polymorphisms investigated in this study. The position and size of the three PCR products in this embodiment of a triplex PCR reaction is indicated below the exons. C. An example of PCR products generated in a triplex PCR reaction as visualized on an agarose gel.

FIGS. 7A-7C shows a table of oligonucleotides used for amplification and INVADER assay detection of CYP 2D6 alleles.

FIG. 8 shows representative data from analysis of exemplary CYP 2D6 alleles using the methods and compositions of the present invention. Each allele tested is indicated at the top of each panel 1 through 5.

FIG. 9 shows a summary of the data from a screen of 174 DNAs with 11 CYP2D6 Invader genotyping assays

FIG. 10 shows CYP2D6 haplotype predictions from 175 genomic samples using the Expectation maximization algorithm implemented on the Arlequin genetic software.

FIG. 11 shows compound CYP2D6 haplotypes for 171 DNAs genotyped by the Invader system and categorized into a number of functional alleles.

FIGS. 12A-12J shows a table of oligonucleotides used for amplification and INVADER assay detection of CYP 2D6 alleles.

FIG. 13 shows clusters of Ratio N values corresponding to copy number for a number of tested samples.

FIG. 14 shows primers pair useful in tetraplex amplification reactions, as well as the size of expected amplification CYP2D6 fragments.

FIG. 15 provides detection assay components for CYP2D6 detection assay in some embodiments of the present invention.

FIG. 16A shows the Net Fold-Over-Zero (FOZ) data of 44 samples tested with the 100C>T INVADER assay. FIG. 16B shows the allele ratios and the genotype calls.

FIG. 17 provides an example of some of the star alleles with a signature SNPs.

FIG. 18 shows examples of some of the star alleles with an exemplary Secondary Signature SNPs.

FIG. 19 provides an exemplary matrix representing all the possible combinations of 29 detection assays and the full genotype of a sample carrying any one of these combinations.

FIG. 20 shows clusters of values corresponding to copy number for a number of tested samples in reflex assay test.

FIG. 21 provides an exemplary matrix of the present invention.

### DEFINITIONS

To facilitate an understanding of the present invention, a number of terms and phrases are defined below:

As used herein, the terms “SNP,” “SNPs” or “single nucleotide polymorphisms” refer to single base changes at a specific location in an organism's (e.g., a human) genome. “SNPs” can be located in a portion of a genome that does not code for a gene. Alternatively, a “SNP” may be located in the coding region of a gene. In this case, the “SNP” may alter the structure and function of the RNA or the protein with which it is associated.

As used herein, the term “allele” refers to a variant form of a given sequence (e.g., including but not limited to, genes containing one or more SNPs). A large number of genes are present in multiple allelic forms in a population. A diploid organism carrying two different alleles of a gene is said to be heterozygous for that gene, whereas a homozygote carries two copies of the same allele.

As used herein, the term “linkage” refers to the proximity of two or more markers (e.g., genes) on a chromosome.

As used herein, the term “allele frequency” refers to the frequency of occurrence of a given allele (e.g., a sequence containing a SNP) in given population (e.g., a specific gender, race, or ethnic group). Certain populations may contain a given allele within a higher percent of its members than other populations. For example, a particular mutation in the breast cancer gene called BRCA1 was found to be present in one percent of the general Jewish population. In comparison, the percentage of people in the general U.S. population that have any mutation in BRCA1 has been estimated to be between 0.1 to 0.6 percent. Two additional mutations, one in the BRCAl gene and one in another breast cancer gene called BRCA2, have a greater prevalence in the Ashkenazi Jewish population, bringing the overall risk for carrying one of these three mutations to 2.3 percent.

As used herein, the term “in silico analysis” refers to analysis performed using computer processors and computer memory. For example, “in silico SNP analysis” refers to the analysis of SNP data using computer processors and memory.

As used herein, the term “genotype” refers to the actual genetic make-up of an organism (e.g., in terms of the particular alleles carried at a genetic locus). Expression of the genotype gives rise to an organism's physical appearance and characteristics—the “phenotype.”

As used herein, the term “locus” refers to the position of a gene or any other characterized sequence on a chromosome.

As used herein the term “disease” or “disease state” refers to a deviation from the condition regarded as normal or average for members of a species, and which is detrimental to an affected individual under conditions that are not inimical to the majority of individuals of that species (e.g., diarrhea, nausea, fever, pain, and inflammation etc).

As used herein, the term “treatment” in reference to a medical course of action refer to steps or actions taken with respect to an affected individual as a consequence of a suspected, anticipated, or existing disease state, or wherein there is a risk or suspected risk of a disease state. Treatment may be provided in anticipation of or in response to a disease state or suspicion of a disease state, and may include, but is not limited to preventative, ameliorative, palliative or curative steps. The term “therapy” refers to a particular course of treatment.

The term “gene” refers to a nucleic acid (e.g., DNA) sequence that comprises coding sequences necessary for the production of a polypeptide, RNA (e.g., rRNA, tRNA, etc.), or precursor. The polypeptide, RNA, or precursor can be encoded by a full length coding sequence or by any portion of the coding sequence so long as the desired activity or functional properties (e.g., ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments included when a gene is transcribed into heterogeneous nuclear RNA (hnRNA); introns may contain regulatory elements such as enhancers. Introns are removed or “spliced out” from the nuclear or primary transcript; introns therefore are generally absent in the messenger RNA (mRNA) transcript. The mRNA functions during translation to specify the sequence or order of amino acids in a nascent polypeptide. Variations (e.g., mutations, SNPS, insertions, deletions) in transcribed portions of genes are reflected in, and can generally be detected in corresponding portions of the produced RNAs (e.g., hnRNAs, mRNAs, rRNAs, tRNAs).

Where the phrase “amino acid sequence” is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. In this case, the DNA sequence thus codes for the amino acid sequence.

DNA and RNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less. “High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polynucleotides, the term “substantial identity” denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a splice variant of the full-length sequences.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q replicase, MDV-1 RNA is the specific template for the replicase (D. L. Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer should be sufficiently long to prime the synthesis of extension products in the presence of the inducing agent. The exact lengths of the primers will depend on many factors, including temperature, source of primer and the use of the method.

As used herein, the term “probe” or “hybridization probe” refers to an oligonucleotide (i.e., a sequence of nucleotides), whether occurring naturally as in a purified restriction digest or produced synthetically, recombinantly or by PCR amplification, that is capable of hybridizing, at least in part, to another oligonucleotide of interest. A probe may be single-stranded or double-stranded. Probes are useful in the detection, identification and isolation of particular sequences. In some preferred embodiments, probes used in the present invention will be labeled with a “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target” refers to a nucleic acid sequence or structure to be detected or characterized.

As used herein, the term “polymerase chain reaction” (“PCR”) refers to the method of K. B. Mullis (See e.g., U.S. Pat. Nos. 4,683,195, 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, “Y target sequences” represents a particular number “Y” of target sequences, wherein “Y” is a numerical value of one or more.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include comprise modified forms of deoxyribonucleotides as well as ribonucleotides.

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acids encoding a polypeptide include, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (e.g., 10 nucleotides, 11, . . . , 20, . . . ).

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of labeled antibodies.

The term “test compound” refers to any chemical entity, pharmaceutical, drug, and the like that are tested in an assay (e.g., a drug screening assay) for any desired activity (e.g., including but not limited to, the ability to treat or prevent a disease, illness, sickness, or disorder of bodily function, or otherwise alter the physiological or cellular status of a sample). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection system known in the art, or combinations thereof.

The term “detection” as used herein refers to quantitatively or qualitatively identifying an analyte (e.g., DNA, RNA or a protein) within a sample. The term “detection assay” as used herein refers to a kit, test, or procedure performed for the purpose of detecting an analyte nucleic acid within a sample. Detection assays produce a detectable signal or effect when performed in the presence of the target analyte, and include but are not limited to assays incorporating the processes of hybridization, nucleic acid cleavage (e.g., exo- or endonuclease), nucleic acid amplification, nucleotide sequencing, primer extension, or nucleic acid ligation.

As used herein, the term “functional detection oligonucleotide” refers to an oligonucleotide that is used as a component of a detection assay, wherein the detection assay is capable of successfully detecting (i.e., producing a detectable signal) an intended target nucleic acid when the functional detection oligonucleotide provides the oligonucleotide component of the detection assay. This is in contrast to a non-functional detection oligonucleotides, which fail to produce a detectable signal in a detection assay for the particular target nucleic acid when the non-functional detection oligonucleotide is provided as the oligonucleotide component of the detection assay. Determining if an oligonucleotide is a functional oligonucleotide can be carried out experimentally by testing the oligonucleotide in the presence of the particular target nucleic acid using the detection assay.

As used herein, the term “derived from a different subject,” such as samples or nucleic acids derived from a different subjects refers to a samples derived from multiple different individuals. For example, a blood sample comprising genomic DNA from a first person and a blood sample comprising genomic DNA from a second person are considered blood samples and genomic DNA samples that are derived from different subjects. A sample comprising five target nucleic acids derived from different subjects is a sample that includes at least five samples from five different individuals. However, the sample may further contain multiple samples from a given individual.

As used herein, the term “treating together”, when used in reference to experiments or assays, refers to conducting experiments concurrently or sequentially, wherein the results of the experiments are produced, collected, or analyzed together (i.e., during the same time period). For example, a plurality of different target sequences located in separate wells of a multiwell plate or in different portions of a microarray are treated together in a detection assay where detection reactions are carried out on the samples simultaneously or sequentially and where the data collected from the assays is analyzed together.

The terms “assay data” and “test result data” as used herein refer to data collected from performance of an assay (e.g., to detect or quantitate a gene, SNP or an RNA). Test result data may be in any form, i.e., it may be raw assay data or analyzed assay data (e.g., previously analyzed by a different process). Collected data that has not been further processed or analyzed is referred to herein as “raw” assay data (e.g., a number corresponding to a measurement of signal, such as a fluorescence signal from a spot on a chip or a reaction vessel, or a number corresponding to measurement of a peak, such as peak height or area, as from, for example, a mass spectrometer, HPLC or capillary separation device), while assay data that has been processed through a further step or analysis (e.g., normalized, compared, or otherwise processed by a calculation) is referred to as “analyzed assay data” or “output assay data”.

As used herein, the term “database” refers to collections of information (e.g., data) arranged for ease of retrieval, for example, stored in a computer memory. A “genomic information database” is a database comprising genomic information, including, but not limited to, polymorphism information (i.e., information pertaining to genetic polymorphisms), genome information (i.e., genomic information), linkage information (i.e., information pertaining to the physical location of a nucleic acid sequence with respect to another nucleic acid sequence, e.g., in a chromosome), and disease association information (i.e., information correlating the presence of or susceptibility to a disease to a physical trait of a subject, e.g., an allele of a subject). “Database information” refers to information to be sent to a databases, stored in a database, processed in a database, or retrieved from a database. “Sequence database information” refers to database information pertaining to nucleic acid sequences. As used herein, the term “distinct sequence databases” refers to two or more databases that contain different information than one another. For example, the dbSNP and GenBank databases are distinct sequence databases because each contains information not found in the other.

As used herein the terms “processor” and “central processing unit” or “CPU” are used interchangeably and refer to a device that is able to read a program from a computer memory (e.g., ROM or other computer memory) and perform a set of steps according to the program.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “hypertext system” refers to a computer-based informational system in which documents (and possibly other types of data entities) are linked together via hyperlinks to form a user-navigable “web.”

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein, the terms “World Wide Web” or “web” refer generally to both (i) a distributed collection of interlinked, user-viewable hypertext documents (commonly referred to as Web documents or Web pages) that are accessible via the Internet, and (ii) the client and server software components which provide user access to such documents using standardized Internet protocols. Currently, the primary standard protocol for allowing applications to locate and acquire Web documents is HTTP, and the Web pages are encoded using HTML. However, the terms “Web” and “World Wide Web” are intended to encompass future markup languages and transport protocols that may be used in place of (or in addition to) HTML and HTTP.

As used herein, the term “web site” refers to a computer system that serves informational content over a network using the standard protocols of the World Wide Web. Typically, a Web site corresponds to a particular Internet domain name and includes the content associated with a particular organization. As used herein, the term is generally intended to encompass both (i) the hardware/software server components that serve the informational content over the network, and (ii) the “back end” hardware/software components, including any non-standard or specialized components, that interact with the server components to perform services for Web site users.

As used herein, the term “HTML” refers to HyperText Markup Language that is a standard coding convention and set of codes for attaching presentation and linking attributes to informational content within documents. HTML is based on SGML, the Standard Generalized Markup Language. During a document authoring stage, the HTML codes (referred to as “tags”) are embedded within the informational content of the document. When the Web document (or HTML document) is subsequently transferred from a Web server to a browser, the codes are interpreted by the browser and used to parse and display the document. Additionally, in specifying how the Web browser is to display the document, HTML tags can be used to create links to other Web documents (commonly referred to as “hyperlinks”).

As used herein, the term “XML” refers to Extensible Markup Language, an application profile that, like HTML, is based on SGML. XML differs from HTML in that: information providers can define new tag and attribute names at will; document structures can be nested to any level of complexity; any XML document can contain an optional description of its grammar for use by applications that need to perform structural validation. XML documents are made up of storage units called entities, which contain either parsed or unparsed data. Parsed data is made up of characters, some of which form character data, and some of which form markup. Markup encodes a description of the document's storage layout and logical structure. XML provides a mechanism to impose constraints on the storage layout and logical structure, to define constraints on the logical structure and to support the use of predefined storage units. A software module called an XML processor is used to read XML documents and provide access to their content and structure.

As used herein, the term “HTTP” refers to HyperText Transport Protocol that is the standard World Wide Web client-server protocol used for the exchange of information (such as HTML documents, and client requests for such documents) between a browser and a Web server. HTTP includes a number of different types of messages that can be sent from the client to the server to request different types of server actions. For example, a “GET” message, which has the format GET, causes the server to return the document or file located at the specified URL.

As used herein, the term “URL” refers to Uniform Resource Locator that is a unique address that fully specifies the location of a file or other resource on the Internet. The general format of a URL is protocol://machine address:port/path/filename. The port specification is optional, and if none is entered by the user, the browser defaults to the standard port for whatever service is specified as the protocol. For example, if HTTP is specified as the protocol, the browser will use the HTTP default port of 80.

As used herein, the term “PUSH technology” refers to an information dissemination technology used to send data to users over a network. In contrast to the World Wide Web (a “pull” technology), in which the client browser should request a Web page before it is sent, PUSH protocols send the informational content to the user computer automatically, typically based on information pre-specified by the user.

As used herein, the term “communication network” refers to any network that allows information to be transmitted from one location to another. For example, a communication network for the transfer of information from one computer to another includes any public or private network that transfers information using electrical, optical, satellite transmission, and the like. Two or more devices that are part of a communication network such that they can directly or indirectly transmit information from one to the other are considered to be “in electronic communication” with one another. A computer network containing multiple computers may have a central computer (“central node”) that processes information to one or more sub-computers that carry out specific tasks (“sub-nodes”). Some networks comprises computers that are in “different geographic locations” from one another, meaning that the computers are located in different physical locations (i.e., aren't physically the same computer, e.g., are located in different countries, states, cities, rooms, etc.).

As used herein, the term “detection assay component” refers to a component of a system capable of performing a detection assay. Detection assay components include, but are not limited to, hybridization probes, buffers, and the like.

As used herein, the term “a detection assays configured for target detection” refers to a collection of assay components that are capable of producing a detectable signal when carried out using the target nucleic acid. For example, a detection assay that has empirically been demonstrated to detect a particular single nucleotide polymorphism is considered a detection assay configured for target detection.

As used herein, the phrase “unique detection assay” refers to a detection assay that has a different collection of detection assay components in relation to other detection assays located on the same detection panel. A unique assay doesn't necessarily detect a different target (e.g. SNP) than other assays on the same detection panel, but it does have a least one difference in the collection of components used to detect a given target (e.g. a unique detection assay may employ a probe sequences that is shorter or longer in length than other assays on the same detection panel).

As used herein, the term “candidate” refers to an assay or analyte, e.g., a nucleic acid, suspected of having a particular feature or property. A “candidate sequence” refers to a nucleic acid suspected of comprising a particular sequence, while a ” candidate oligonucleotide” refers to an oligonucleotide suspected of having a property such as comprising a particular sequence, or having the capability to hybridize to a target nucleic acid or to perform in a detection assay. A “candidate detection assay” refers to a detection assay that is suspected of being a valid detection assay.

As used herein, the term “detection panel” refers to a substrate or device containing at least two unique candidate detection assays configured for target detection.

As used herein, the term “valid detection assay” refers to a detection assay that has been shown to accurately predict an association between the detection of a target and a phenotype (e.g. medical condition). Examples of valid detection assays include, but are not limited to, detection assays that, when a target is detected, accurately predict the phenotype medical 95%, 96%, 97%, 98%, 99%, 99.5%, 99.8%, or 99.9% of the time. Other examples of valid detection assays include, but are not limited to, detection assays that quality as and/or are marketed as Analyte-Specific Reagents (i.e. as defined by FDA regulations) or In-Vitro Diagnostics (i.e. approved by the FDA).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc. “Allele frequency information” refers to facts or data pertaining allele frequencies, including, but not limited to, allele identities, statistical correlations between the presence of an allele and a characteristic of a subject (e.g., a human subject), the presence or absence of an allele in a individual or population, the percentage likelihood of an allele being present in an individual having one or more particular characteristics, etc.

As used herein, the term “assay validation information” refers to genomic information and/or allele frequency information resulting from processing of test result data (e.g. processing with the aid of a computer). Assay validation information may be used, for example, to identify a particular candidate detection assay as a valid detection assay.

### DETAILED DESCRIPTION OF THE INVENTION

The following discussion provides a description of certain preferred illustrative embodiments of the present invention and is not intended to limit the scope of the present invention.

I. Detection of CYP2D6 Genotypes

The present invention provides comprehensive systems and methods for the characterization of CYP2D6 genotypes. For example, the present invention provides systems and methods of characterizing both the identity of polymorphisms in and around the CYP2D6 gene, as well as copy number of either or both the CYP2D6 gene and genic regions or portions thereof to characterize individuals as having a particular CYP2D6 genotype. An understanding of the specific genotypes of a subject or subjects facilitates more rational therapeutic interventions, design of drug trials, and basic research into genotype/phenotype correlations. Systems and methods for comprehensive analysis of CYP2DG genotypes are provided in detail in Examples 3 and 4, below, in addition to the corresponding figures.

More than 50 variants of CYP2D6 are known (Marez et al., Pharmacogenetics 7:193-202 (1997)). The accepted nomenclature labels groups of polymorphisms that occur together (Daly et al., Pharmacogenetics 6:193-201 (1996)). The gene name is followed by a * and a number (example CYP2D6*2). These groups of polymorphisms are called alleles. As more polymorphisms were discovered, many overlapped with already identified alleles. In these cases, the alleles were given an alphabetic character following the numerical character (example CYP2D6*2A and CYP2D6*2B). These are called sub-alleles. The overlap of polymorphisms between alleles and sub-alleles contributes to the complexity of correlating the genotype of a polymorphism with the allele designation. This is especially true with CYP2D6*2.

While most alleles and sub-alleles have one polymorphism which is unique to the group and can be used as a “signature” to determine the allele or sub-allele type, this is not the case for CYP2D6*2. CYP2D6*2 is composed of 10 sub-alleles e.g. *2A, 2B, . . . 2K. The most common polymorphisms that are present in all 10 sub-alleles of *2 are 2850C>T and 4180G>C. In addition, these two polymorphisms are also present in 14 other alleles of CYP2D6, e.g., *4, *8, * and *11. Therefore, to characterize a sample as having a *2 allele designation, the genotype of the signature polymorphisms for the 14 other alleles should be determined. There are 16 signature polymorphisms for these 14 alleles. In summary, a total of 18 mutations are useful to characterize a sample as having a *2 allele designation; the two signature polymorphisms for *2 and the 16 signature polymorphisms for the 14 alleles which also carry the signatures for *2.

II. Multiplex PCR Primer Design

The INVADER assay can be used for the detection of single nucleotide polymorphisms (SNPs) with as little as 10-100 ng of genomic DNA without the need for target pre-amplification. However, with more than 50,000 INVADER assays being developed and the potential for whole genome association studies involving hundreds of thousands of SNPs, the amount of sample DNA becomes a limiting factor for large scale analysis. Due to the sensitivity of the INVADER assay on human genomic DNA (hgDNA) without target amplification, multiplex PCR coupled with the INVADER assay requires only limited target amplification (10³-10⁴ ) as compared to typical multiplex PCR reactions which require extensive amplification (10⁹-10¹²) for conventional gel detection methods. The low level of target amplification used for INVADER™ detection provides for more extensive multiplexing by avoiding amplification inhibition commonly resulting from target accumulation.

The present invention provides methods and selection criteria that allow primer sets for multiplex PCR to be generated (e.g. that can be coupled with a detection assay, such as the INVADER assay). In some embodiments, software applications of the present invention automated multiplex PCR primer selection, thus allowing highly multiplexed PCR with the primers designed thereby. Using the INVADER Medically Associated Panel (MAP) as a corresponding platform for SNP detection, as shown in example 2, the methods, software, and selection criteria of the present invention allowed accurate genotyping of 94 of the 101 possible amplicons (˜93%) from a single PCR reaction. The original PCR reaction used only 10 ng of hgDNA as template, corresponding to less than 150 pg hgDNA per INVADER assay.

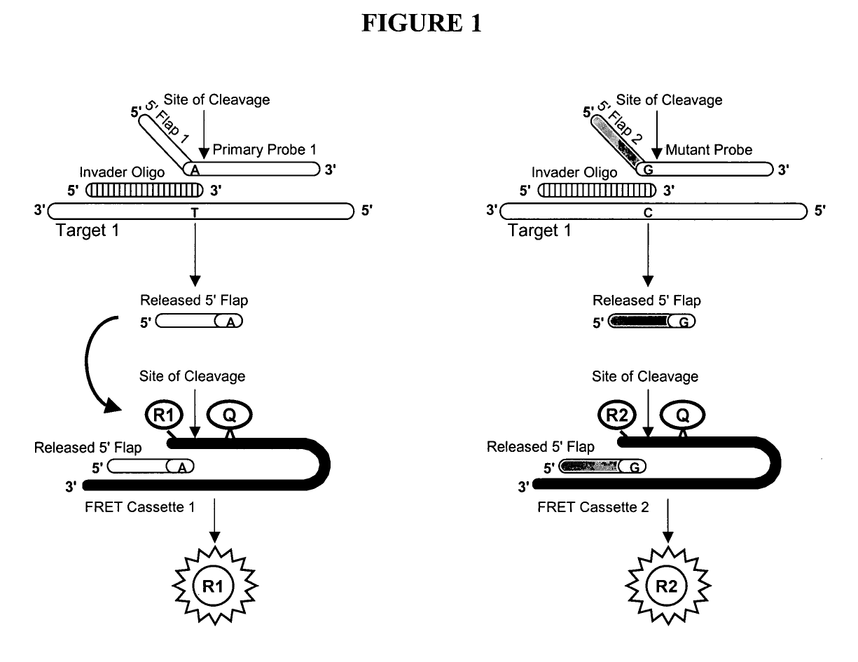

FIG. 1 described the general principles of the INVADER assay. The NVADER assay allows for the simultaneous detection of two distinct alleles in the same reaction using an isothermal, single addition format. (A) Allele discrimination takes place by “structure specific” cleavage of the Probe, releasing a 5′ flap which corresponds to a given polymorphism. (B) In the second reaction, the released 5′ flap mediates signal generation by cleavage of the appropriate FRET cassette.

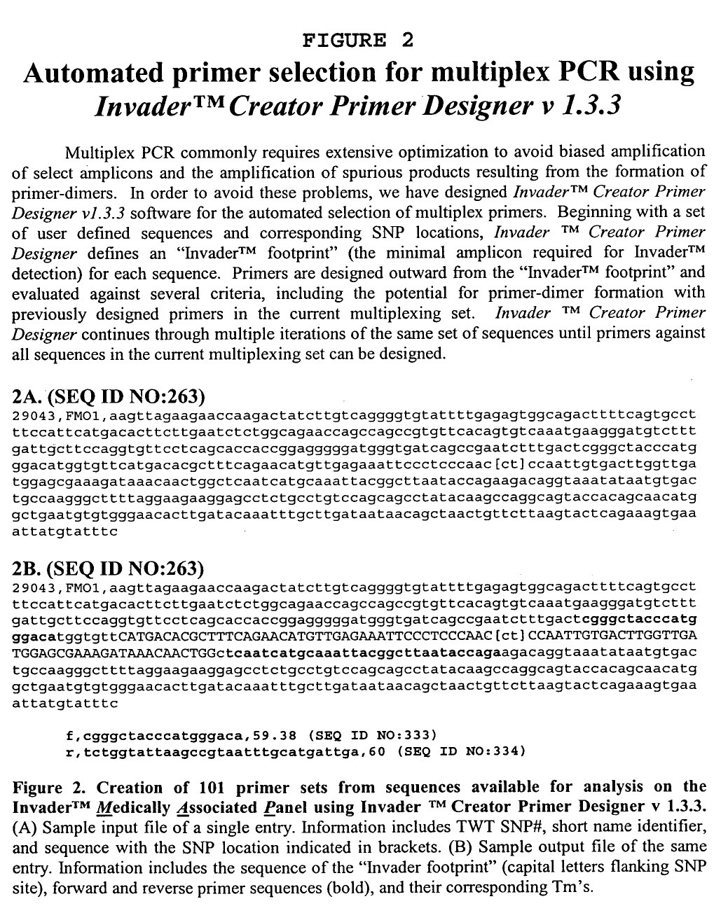

FIG. 2 illustrates creation of one of the primer pairs (both a forward and reverse primer) for a 101 primer sets from sequences available for analysis on the INVADER Medically Associated Panel using one embodiment of the software application of the present invention. FIG. 2A shows a sample input file of a single entry (e.g. shows target sequence information for a single target sequence containing a SNP that is processed the method and software of the present invention). The target sequence information in FIG. 2 includes Third Wave Technologies's SNP#, short name identifier, and sequence with the SNP location indicated in brackets. FIG. 2B shows the sample output file of a the same entry (e.g. shows the target sequence after being processed by the systems and methods and software of the present invention. The output information includes the sequence of the footprint region (capital letters flanking SNP site, showing region where INVADER assay probes hybridize to this target sequence in order to detect the SNP in the target sequence), forward and reverse primer sequences (bold), and their corresponding Tm's.

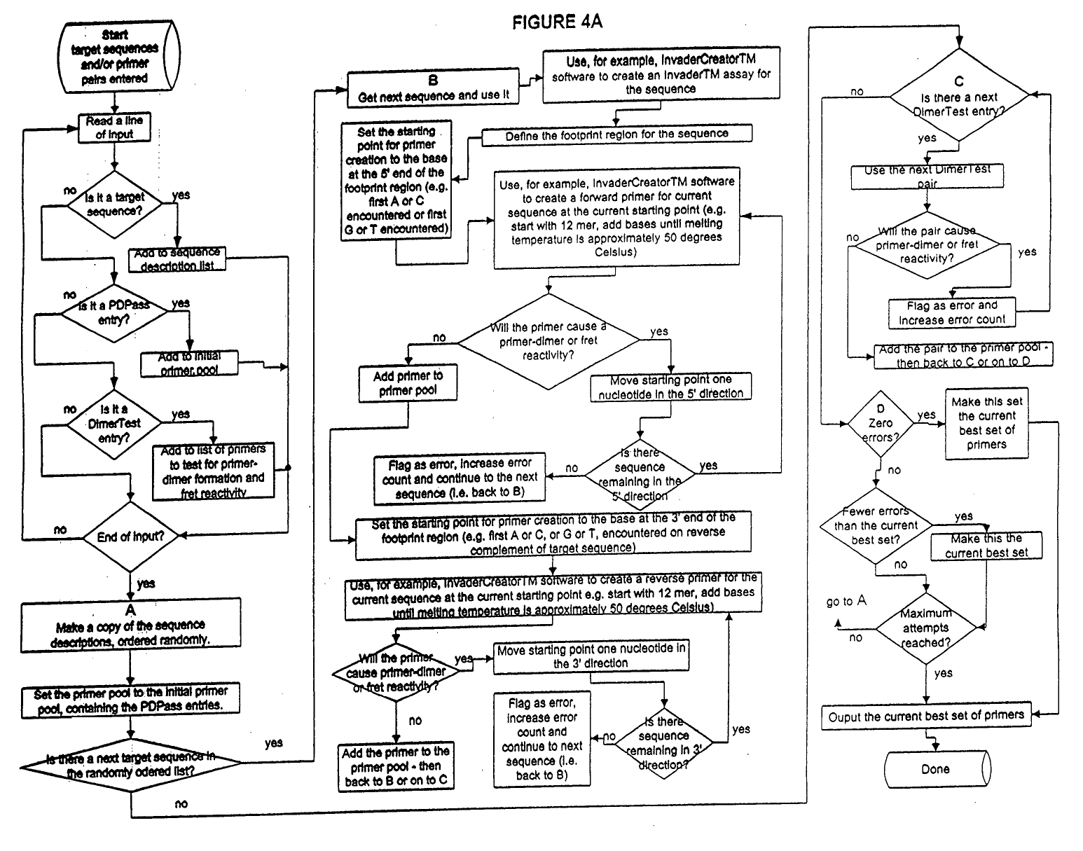

In some embodiments, the selection of primers to make a primer set capable of multiplex PCR is performed in automated fashion (e.g. by a software application). Automated primer selection for multiplex PCR may be accomplished employing a software program designed as shown by the flow chart in FIG. 4A.

Multiplex PCR commonly requires extensive optimization to avoid biased amplification of select amplicons and the amplification of spurious products resulting from the formation of primer-dimers. In order to avoid these problems, the present invention provides methods and software application that provide selection criteria to generate a primer set configured for multiplex PCR, and subsequent use in a detection assay (e.g. INVADER detection assays).

In some embodiments, the methods and software applications of the present invention start with user defined sequences and corresponding SNP locations. In certain embodiments, the methods and/or software application determines a footprint region within the target sequence (the minimal amplicon required for INVADER assay detection) for each sequence (shown in capital letters in FIG. 2B). The footprint region includes the region where assay probes hybridize, as well as any user defined additional bases extending outward therefore (e.g. 5 additional bases included on each side of where the assay probes hybridize). Next, primers are designed outward from the footprint region and evaluated against several criteria, including the potential for primer-dimer formation with previously designed primers in the current multiplexing set (See, primers in bold in FIG. 2A, and selection steps in FIG. 4A). This process may be continued, as shown in FIG. 4A, through multiple iterations of the same set of sequences until primers against all sequences in the current multiplexing set can be designed.

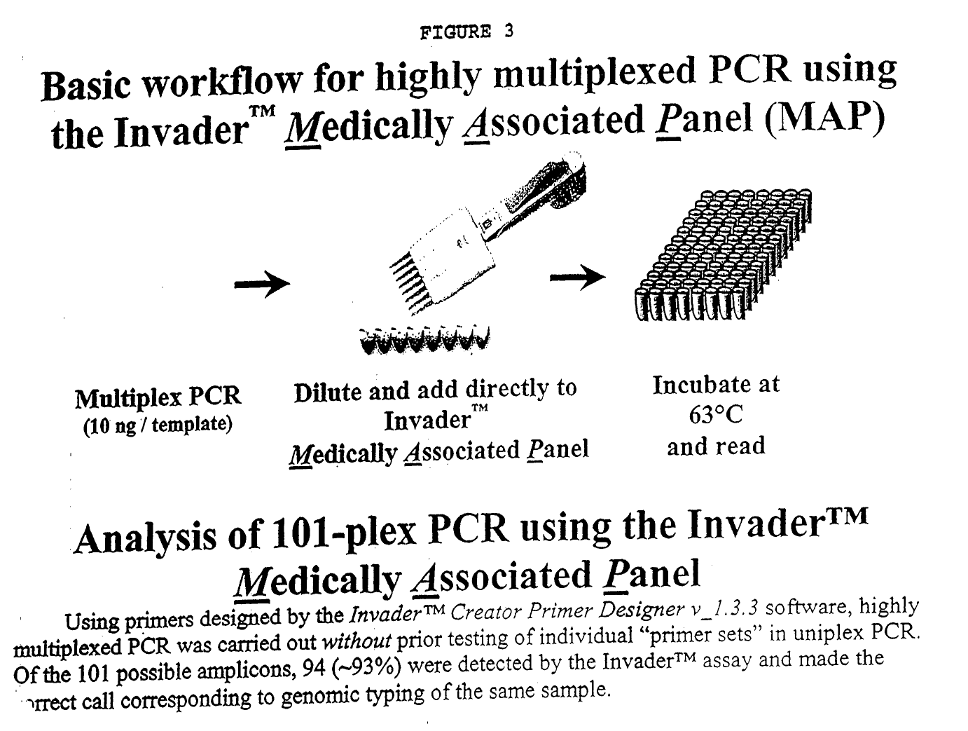

Once a primer set is designed for multiplex PCR, this set may be employed as shown in the basic workflow scheme shown in FIG. 3. Multiplex PCR may be carried out, for example, under standard conditions using only 10 ng of hgDNA as template. After 10 min at 95° C., Taq (2.5 units) may be added to a 50 ul reaction and PCR carried out for 50 cycles. The PCR reaction may be diluted and loaded directly onto an INVADER MAP plate (3 ul/well) (See FIG. 3). An additional 3 ul of 15 mM MgCl₂ may be added to each reaction on the INVADER MAP plate and covered with 6 ul of mineral oil. The entire plate may then be heated to 95° C. for 5 min. and incubated at 63° C. for 40 min. FAM and RED fluorescence may then be measured on a Cytofluor 4000 fluorescent plate reader and “Fold Over Zero” (FOZ) values calculated for each amplicon. Results from each SNP may be color coded in a table as “pass” (green), “mis-call” (pink), or “no-call” (white) (See, Example 2 below).

In some embodiments the number of PCR reactions is from about 1 to about 10 reactions. In some embodiments, the number of PCR reactions is from about 10 to about 50 reactions. In further embodiments, the number of PCR reactions is from about 50 to about 100. In additional embodiments, the number of PCR reactions is greater than 100.

The present invention also provides methods to optimize multiplex PCR reactions (e.g. once a primer set is generated, the concentration of each primer or primer pair may be optimized). For example, once a primer set has been generated and used in a multiplex PCR at equal molar concentrations, the primers may be evaluated separately such that the optimum primer concentration is determined such that the multiplex primer set performs better.

Multiplex PCR reactions are being recognized in the scientific, research, clinical and biotechnology industries as potentially time effective and less expensive means of obtaining nucleic acid information compared to standard, monoplex PCR reactions. Instead of performing only a single amplification reaction per reaction vessel (tube or well of a multi-well plate for example), numerous amplification reactions are performed in a single reaction vessel. The cost per target is theoretically lowered by eliminating technician time in assay set-up and data analysis, and by the substantial reagent savings (especially enzyme cost). Another benefit of the multiplex approach is that far less target sample is required. In whole genome association studies involving hundreds of thousands of single nucleotide polymorphisms (SNPs), the amount of target or test sample is limiting for large scale analysis, so the concept of performing a single reaction, using one sample aliquot to obtain, for example, 100 results, versus using 100 sample aliquots to obtain the same data set is an attractive option.

To design primers for a successful multiplex PCR reaction, the issue of aberrant interaction among primers should be addressed. The formation of primer dimers, even if only a few bases in length, may inhibit both primers from correctly hybridizing to the target sequence. Further, if the dimers form at or near the 3′ ends of the primers, no amplification or very low levels of amplification will occur, since the 3′ end is required for the priming event. Clearly, the more primers utilized per multiplex reaction, the more aberrant primer interactions are possible. The methods, systems and applications of the present help prevent primer dimers in large sets of primers, making the set suitable for highly multiplexed PCR.

When designing primer pairs for numerous site (for example 100 sites in a multiplex PCR reaction), the order in which primer pairs are designed can influence the total number of compatible primer pairs for a reaction. For example, if a first set of primers is designed for a first target region that happens to be an A/T rich target region, these primer will be A/T rich. If the second target region chosen also happens to be an A/T rich target region, it is far more likely that the primers designed for these two sets will be incompatible due to aberrant interactions, such as primer dimers. If, however, the second target region chosen is not A/T rich, it is much more likely that a primer set can be designed that will not interact with the first A/T rich set. For any given set of input target sequences, the present invention randomizes the order in which primer sets are designed (See, FIG. 4A). Furthermore, in some embodiments, the present invention re-orders the set of input target sequences in a plurality of different, random orders to maximize the number of compatible primer sets for any given multiplex reaction (See, FIG. 4A).

The present invention provides criteria for primer design which minimizes 3′ interactions while maximizing the number of compatible primer pairs for a given set of reaction targets in a multiplex design. For primers described as 5′-N[x]-N[x-1]- . . . -N[4]-N[3]-N[2]-N[1]-3′, N[1] is an A or C (in alternative embodiments, N[1] is a G or T). N[2]-N[1] of each of the forward and reverse primers designed should not be complementary to N[2]-N[1] of any other oligonucleotide. In certain embodiments, N[3]-N[2]-N[1] should not be complementary to N[3]-N[2]-N[1] of any other oligonucleotide. In preferred embodiments, if these criteria are not met at a given N[1], the next base in the 5′ direction for the forward primer or the next base in the 3′ direction for the reverse primer may be evaluated as an N[1] site. This process is repeated, in conjunction with the target randomization, until all criteria are met for all, or a large majority of, the targets sequences (e.g. 95% of target sequences can have primer pairs made for the primer set that fulfill these criteria).

Another challenge to be overcome in a multiplex primer design is the balance between actual, required nucleotide sequence, sequence length, and the oligonucleotide melting temperature (Tm) constraints. Importantly, since the primers in a multiplex primer set in a reaction should function under the same reaction conditions of buffer, salts and temperature, they need therefore to have substantially similar Tm's, regardless of GC or AT richness of the region of interest. The present invention allows for primer design which meet minimum Tm and maximum Tm requirements and minimum and maximum length requirements. For example, in the formula for each primer 5′-N[x]-N[x-1]- . . . -N[4]-N[3]-N[2]-N[1]-3′, x is selected such the primer has a predetermined melting temperature (e.g. bases are included in the primer until the primer has a calculated melting temperature of about 50 degrees Celsius).

Often the products of a PCR reaction are used as the target material for another nucleic acid detection means, such as a hybridization-type detection assays, or the INVADER reaction assays for example. Consideration should be given to the location of primer placement to allow for the secondary reaction to successfully occur, and again, aberrant interactions between amplification primers and secondary reaction oligonucleotides should be minimized for accurate results and data. Selection criteria may be employed such that the primers designed for a multiplex primer set do not react (e.g. hybridize with, or trigger reactions) with oligonucleotide components of a detection assay. For example, in order to prevent primers from reacting with the FRET oligonucleotide of a bi-plex INVADER assay, certain homology criteria is employed. In particular, if each of the primers in the set are defined as 5′-N[x]-N[x-1]- . . . -N[4]-N[3]-N[2]-N[1]-3′, then N[4]-N[3]-N[2]-N[1]-3′ is selected such that it is less than 90% homologous with the FRET or INVADER oligonucleotides. In other embodiments, N[4]-N[3]-N[2]-N[1]-3′ is selected for each primer such that it is less than 80% homologous with the FRET or INVADER oligonucleotides. In certain embodiments, N[4]-N[3]-N[2]-N[1]-3′ is selected for each primer such that it is less than 70% homologous with the FRET or INVADER oligonucleotides.

While employing the criteria of the present invention to develop a primer set, some primer pairs may not meet all of the stated criteria (these may be rejected as errors). For example, in a set of 100 targets, 30 are designed and meet all listed criteria, however, set 31 fails. In the method of the present invention, set 31 may be flagged as failing, and the method could continue through the list of 100 targets, again flagging those sets which do not meet the criteria (See FIG. 4A). Once all 100 targets have had a chance at primer design, the method would note the number of failed sets, re-order the 100 targets in a new random order and repeat the design process (See, FIG. 4A). After a configurable number of runs, the set with the most passed primer pairs (the least number of failed sets) are chosen for the multiplex PCR reaction (See FIG. 4A).

FIG. 4A shows a flow chart with the basic flow of certain embodiments of the methods and software application of the present invention. In preferred embodiments, the processes detailed in FIG. 4A are incorporated into a software application for ease of use (although, the methods may also be performed manually using, for example, FIG. 4A as a guide).

Target sequences and/or primer pairs are entered into the system shown in FIG. 4A. The first set of boxes show how target sequences are added to the list of sequences that have a footprint determined (See “B” in FIG. 4A), while other sequences are passed immediately into the primer set pool (e.g. PDPass, those sequences that have been previously processed and shown to work together without forming Primer dimers or having reactivity to FRET sequences), as well as DimerTest entries (e.g. pair or primers a user wants to use, but that has not been tested yet for primer dimer or fret reactivity). In other words, the initial set of boxes leading up to “end of input” sort the sequences so they can be later processed properly.

Starting at “A” in FIG. 4A, the primer pool is basically cleared or “emptied” to start a fresh run. The target sequences are then sent to “B” to be processed, and DimerTest pairs are sent to “C” to be processed. Target sequences are sent to “B”, where a user or sofware application determines the footprint region for the target sequence (e.g. where the assay probes will hybridize in order to detect the mutation (e.g. SNP) in the target sequence). This region is generally shown in capital letters in figures, such as FIG. 2B. It is important to design this region (which the user may further expand by defining that additional bases past the hybridization region be added) such that the primers that are designed fully encompass this region. In FIG. 4A, the software application INVADER CREATOR is used to design the INVADER oligonuclotide and downstream probes that will hybridize with the target region (although any type of program of system could be used to create any type of probes a user was interested in designing probes for, and thus determining the footprint region for on the target sequence). Thus the core footprint region is then defined by the location of these two assay probes on the target.

Next, the system starts from the 5′ edge of the footprint and travels in the 5′ direction until the first base is reached, or until the first A or C (or G or T) is reached. This is set as the initial starting point for defining the sequence of the forward primer (i.e. this serves as the initial N[1] site). From this initial N[1] site, the sequence of the primer for the forward primer is the same as those bases encountered on the target region. For example, if the default size of the primer is set as 12 bases, the system starts with the bases selected as N[1] and then adds the next 11 bases found in the target sequences. This 12-mer primer is then tested for a melting temperature (e.g. using INVADER CREATOR), and additional bases are added from the target sequence until the sequence has a melting temperature that is designated by the user (e.g. about 50 degrees Celsius, and not more than 55 degrees Celsius). For example, the system employs the formula 5′-N[x]-N[x-1]- . . . -N[4]-N[3]-N[2]-N[1]-3′, and x is initially 12. Then the system adjusts x to a higher number (e.g. longer sequences) until the pre-set melting temperature is found.

The next box in FIG. 4a, is used to determine if the primer that has been designed so far will cause primer-dimer and/or fret reactivity (e.g. with the other sequences already in the pool). The criteria used for this determination are explained above. If the primer passes this step, the forward primer is added to the primer pool. However, if the forward primer fails this criteria, as shown in FIG. 4A, the starting point (N[1] is moved) one nucleotide in the 5′ direction (or to the next A or C, or next G or T). The system first checks to make sure shifting over leaves enough room on the target sequence to successfully make a primer. If yes, the system loops back and check this new primer for melting temperature. However, if no sequence can be designed, then the target sequence is flagged as an error (e.g. indicating that no forward primer can be made for this target).

This same process is then repeated for designing the reverse primer, as shown in FIG. 4A. If a reverse primer is successfully made, then the pair or primers is put into the primer pool, and the system goes back to “B” (if there are more target sequences to process), or goes onto “C” to test DimerTest pairs.

Starting a “C” in FIG. 4A shows how primer pairs that are entered as primers (DimerTest) are processed by the system. If there are no DimerTest pairs, as shown in FIG. 4a, the system goes on to “D”. However, if there are DimerTest pairs, these are tested for primer-dimer and/or FRET reactivity as described above. If the DimerTest pair fails these criteria they are flagged as errors. If the DimerTest pair passes the criteria, they are added to the primer set pool, and then the system goes back to “C” if there are more DimerTest pairs to be evaluated, or or goes on to “D” if there are no more DimerTest pairs to be evaluated.

Starting at “D” in FIG. 4a, the pool of primers that has been created is evaluated. The first step in this section is to examine the number of error (failures) generated by this particular randomized run of sequences. If there were no errors, this set is the best set as maybe ouputted to a user. If there are more than zero errors, the system compares this run to any other previous runs to see what run resulted in the fewest errors. If the current run has fewer errors, it is designated as the current best set. At this point, the system may go back to “A” to start the run over with another randomized set of the same sequences, or the pre-set maximum number of runs (e.g. 5 runs) may have been reached on this run (e.g. this was the 5th run, and the maximum number of runs was set as 5). If the maximum has been reached, then the best set is outputted as the best set. This best set of primers may then be used to generate as physical set of oligonucleotides such that a multiplex PCR reaction may be carried out.

Another challenge to be overcome with multiplex PCR reactions is the unequal amplicon concentrations that result in a standard multiplex reaction. The different loci targeted for amplification may each behave differently in the amplification reaction, yielding vastly different concentrations of each of the different amplicon products. The present invention provides methods, systems, software applications, computer systems, and a computer data storage medium that may be used to adjust primer concentrations relative to a first detection assay read (e.g. INVADER assay read), and then with balanced primer concentrations come close to substantially equal concentrations of different amplicons.

The concentrations for various primer pairs may be determined experimentally. In some embodiments, there is a first run conducted with all of the primers in equimolar concentrations. Time reads are then conducted. Based upon the time reads, the relative amplification factors for each amplicon are determined. Then based upon a unifying correction equation, an estimate of what the primer concentration should be obtained to get the signals closer within the same time point. These detection assays can be on an array of different sizes (384 well plates).

It is appreciated that combining the invention with detection assays and arrays of detection assays provides substantial processing efficiencies. Employing a balanced mix of primers or primer pairs created using the invention, a single point read can be carried out so that an average user can obtain great efficiencies in conducting tests that require high sensitivity and specificity across an array of different targets.

Having optimized primer pair concentrations in a single reaction vessel allows the user to conduct amplification for a plurality or multiplicity of amplification targets in a single reaction vessel and in a single step. The yield of the single step process is then used to successfully obtain test result data for, for example, several hundred assays. For example, each well on a 384 well plate can have a different detection assay thereon. The results of the single step mutliplex PCR reaction has amplified 384 different targets of genomic DNA, and provides you with 384 test results for each plate. Where each well has a plurality of assays even greater efficiencies can be obtained.

Therefore, the present invention provides the use of the concentration of each primer set in highly multiplexed PCR as a parameter to achieve an unbiased amplification of each PCR product. Any PCR includes primer annealing and primer extension steps. Under standard PCR conditions, high concentration of primers in the order of 1 uM ensures fast kinetics of primers annealing while the optimal time of the primer extension step depends on the size of the amplified product and can be much longer than the annealing step. By reducing primer concentration, the primer annealing kinetics can become a rate limiting step and PCR amplification factor should strongly depend on primer concentration, association rate constant of the primers, and the annealing time.

The binding of primer P with target T can be described by the following model: 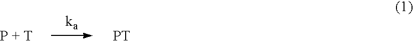
where kₐ is the association rate constant of primer annealing. We assume that the annealing occurs at the temperatures below primer melting and the reverse reaction can be ignored. The solution for this kinetics under the conditions of a primer excess is well known:
[PT]=T₀(1−e^{-k}^{ₐ}^{ct}) (2)
where [PT] is the concentration of target molecules associated with primer, T₀ is initial target concentration, c is the initial primer concentration, and t is primer annealing time. Assuming that each target molecule associated with primer is replicated to produce full size PCR product, the target amplification factor in a single PCR cycle is

$$Z = \frac{{T}_{0} + [ PT ]}{{T}_{0}} = 2 - {ⅇ}^{- {k}_{a} ct} \qquad ( 3 )$$

The total PCR amplification factor after n cycles is given by
F=Zⁿ=(2−e^{-k}^{ₐ}^{ct})ⁿ (4)

As it follows from equation 4, under the conditions where the primer annealing kinetics is the rate limiting step of PCR, the amplification factor should strongly depend on primer concentration. Thus, biased loci amplification, whether it is caused by individual association rate constants, primer extension steps or any other factors, can be corrected by adjusting primer concentration for each primer set in the multiplex PCR. The adjusted primer concentrations can be also used to correct biased performance of INVADER assay used for analysis of PCR pre-amplified loci. Employing this basic principle, the present invention has demonstrated a linear relationship between amplification efficiency and primer concentration and used this equation to balance primer concentrations of different amplicons, resulting in the equal amplification of ten different amplicons in Example 1. This technique may be employed on any size set of multiplex primer pairs.

III. Detection Assay Design

The following section describes detection assays that may be employed with the present invention. For example, many different assays may be used to determine the footprint on the target nucleic sequence, and then used as the detection assay run on the output of the multiplex PCR (or the detection assays may be run simultaneously with the multiplex PCR reaction).

There are a wide variety of detection technologies available for determining the sequence of a target nucleic acid at one or more locations. For example, there are numerous technologies available for detecting the presence or absence of SNPs. Many of these techniques require the use of an oligonucleotide to hybridize to the target. Depending on the assay used, the oligonucleotide is then cleaved, elongated, ligated, disassociated, or otherwise altered, wherein its behavior in the assay is monitored as a means for characterizing the sequence of the target nucleic acid.

The present invention provides systems and methods for the design of oligonucleotides for use in detection assays. In particular, the present invention provides systems and methods for the design of oligonucleotides that successfully hybridize to appropriate regions of target nucleic acids (e.g., regions of target nucleic acids that do not contain secondary structure) under the desired reaction conditions (e.g., temperature, buffer conditions, etc.) for the detection assay. The systems and methods also allow for the design of multiple different oligonucleotides (e.g., oligonucleotides that hybridize to different portions of a target nucleic acid or that hybridize to two or more different target nucleic acids) that all function in the detection assay under the same or substantially the same reaction conditions. These systems and methods may also be used to design control samples that work under the experimental reaction conditions.

While the systems and methods of the present invention are not limited to any particular detection assay, the following description illustrates the invention when used in conjunction with the INVADER assay (Third Wave Technologies, Madison Wis.; See e.g., U.S. Pat. Nos. 5,846,717, 5,985,557, 5,994,069, and 6,001,567 and PCT Publications WO 97/27214 and WO 98/42873, Lyamichev et al., Nat. Biotech., 17:292 (1999), Hall et al., PNAS, USA, 97:8272 (2000), incorporated herein by reference in their entireties) to detect a SNP. The INVADER assay provides ease-of-use and sensitivity levels that, when used in conjunction with the systems and methods of the present invention, find use in detection panels, ASRs, and clinical diagnostics. One skilled in the art will appreciate that specific and general features of this illustrative example are generally applicable to other detection assays.

A. INVADER Assay

The INVADER assay provides means for forming a nucleic acid cleavage structure that is dependent upon the presence of a target nucleic acid and cleaving the nucleic acid cleavage structure so as to release distinctive cleavage products. 5′ nuclease activity, for example, is used to cleave the target-dependent cleavage structure and the resulting cleavage products are indicative of the presence of specific target nucleic acid sequences in the sample. When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced.

The INVADER assay provides detections assays in which the target nucleic acid is reused or recycled during multiple rounds of hybridization with oligonucleotide probes and cleavage of the probes without the need to use temperature cycling (i.e., for periodic denaturation of target nucleic acid strands) or nucleic acid synthesis (i.e., for the polymerization-based displacement of target or probe nucleic acid strands). When a cleavage reaction is run under conditions in which the probes are continuously replaced on the target strand (e.g. through probe-probe displacement or through an equilibrium between probe/target association and disassociation, or through a combination comprising these mechanisms, (Reynaldo, et al., J. Mol. Biol. 97: 511-520 [2000]), multiple probes can hybridize to the same target, allowing multiple cleavages, and the generation of multiple cleavage products.

B. Oligonucleotide Design for the INVADER assay

In some embodiments where an oligonucleotide is designed for use in the INVADER assay to detect a SNP, the sequence(s) of interest are entered into the INVADERCREATOR program (Third Wave Technologies, Madison, Wis.). As described above, sequences may be input for analysis from any number of sources, either directly into the computer hosting the INVADERCREATOR program, or via a remote computer linked through a communication network (e.g., a LAN, Intranet or Internet network). The program designs probes for both the sense and antisense strand. Strand selection is generally based upon the ease of synthesis, minimization of secondary structure formation, and manufacturability. In some embodiments, the user chooses the strand for sequences to be designed for. In other embodiments, the software automatically selects the strand. By incorporating thermodynamic parameters for optimum probe cycling and signal generation (Allawi and SantaLucia, Biochemistry, 36:10581 [1997]), oligonucleotide probes may be designed to operate at a pre-selected assay temperature (e.g., 63° C.). Based on these criteria, a final probe set (e.g., primary probes for 2 alleles and an INVADER oligonucleotide) is selected.

In some embodiments, the INVADERCREATOR system is a web-based program with secure site access that contains a link to BLAST (available at the National Center for Biotechnology Information, National Library of Medicine, National Institutes of Health website) and that can be linked to RNAstructure (Mathews et al., RNA 5:1458 [1999]), a software program that incorporates mfold (Zuker, Science, 244:48 [1989]). RNAstructure tests the proposed oligonucleotide designs generated by INVADERCREATOR for potential uni- and bimolecular complex formation. INVADERCREATOR is open database connectivity (ODBC)-compliant and uses the Oracle database for export/integration. The INVADERCREATOR system was configured with Oracle to work well with UNIX systems, as most genome centers are UNIX-based.

In some embodiments, the INVADERCREATOR analysis is provided on a separate server (e.g., a Sun server) so it can handle analysis of large batch jobs. For example, a customer can submit up to 2,000 SNP sequences in one email. The server passes the batch of sequences on to the INVADERCREATOR software, and, when initiated, the program designs detection assay oligonucleotide sets. In some embodiments, probe set designs are returned to the user within 24 hours of receipt of the sequences.

Each INVADER reaction includes at least two target sequence-specific, unlabeled oligonucleotides for the primary reaction: an upstream INVADER oligonucleotide and a downstream Probe oligonucleotide. The INVADER oligonucleotide is generally designed to bind stably at the reaction temperature, while the probe is designed to freely associate and disassociate with the target strand, with cleavage occurring only when an uncut probe hybridizes adjacent to an overlapping INVADER oligonucleotide. In some embodiments, the probe includes a 5′ flap or “arm” that is not complementary to the target, and this flap is released from the probe when cleavage occurs. In some embodiments, the released flap participates as an INVADER oligonucleotide in a secondary reaction.

The present invention is not limited to the use of the INVADERCREATOR software. Indeed, a variety of software programs are contemplated and are commercially available, including, but not limited to GCG Wisconsin Package (Accelrys, San Diego, Calif.) and Vector NTI (Informax, Rockville, Md.).

Other detection assays may be used in the present invention.

1. Direct Sequencing Assays

In some embodiments of the present invention, variant sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

2. PCR Assay

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

3. Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type.

a. RFLP Assay

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are generally separated by gel electrophoresis and may be visualized by ethidium bromide staining. The length of the fragments is compared to molecular weight markers and fragments generated from wild-type and mutant controls.

b. CFLP Assay

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thernostable nuclease that recognizes and cleaves the junctions between these single-stranded and double-stranded regions.

The region of interest is first isolated, for example, using PCR. In preferred emodiments, one or both strands are labeled. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by denaturing gel electrophoresis) and visualized (e.g., by autoradiography, fluorescence imaging or staining). The length of the fragments is compared to molecular weight markers and fragments generated from wild-type and mutant controls.

4. Hybridization Assays

In preferred embodiments of the present invention, variant sequences are detected a hybridization assay. In a hybridization assay, the presence of absence of a given SNP or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY [1991]). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.

b. Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin).

### EXAMPLES

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); DS (dextran sulfate); C. (degrees Centigrade); and Sigma (Sigma Chemical Co., St. Louis, Mo.); Z28 refers to ECLIPSE dark quencher (Epoch Biosciences, Bothell Wash.).

### Example 1

### A. Designing A 10-plex (Manual): Test for Invader Assays

The following experimental example describes the manual design of amplification primers for a multiplex amplification reaction, and the subsequent detection of the amplicons by the INVADER assay. These data are additionally described in U.S. patent application Ser. No. 10/321,039, filed Dec. 17, 2002, incorporated herein by reference.

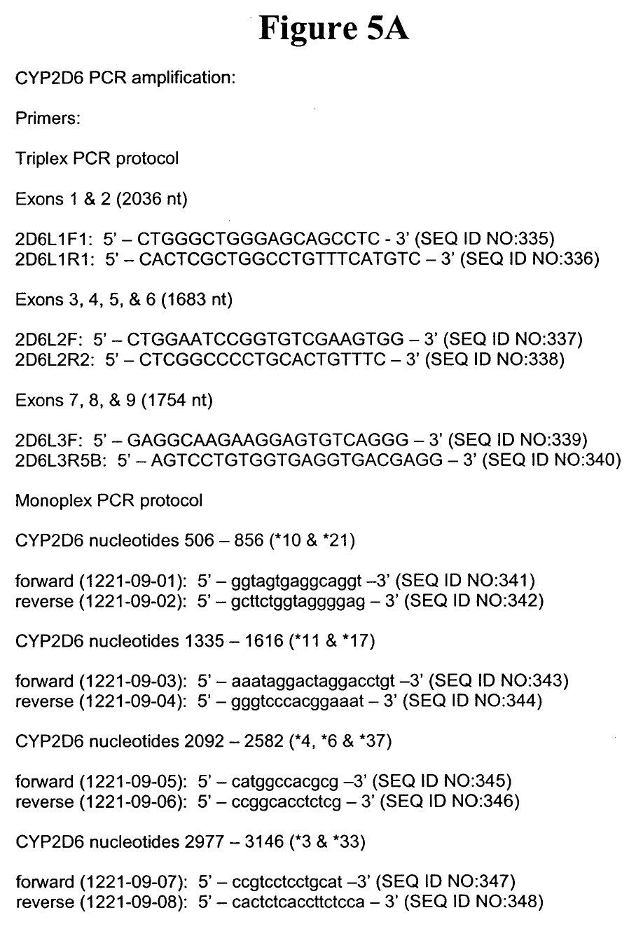

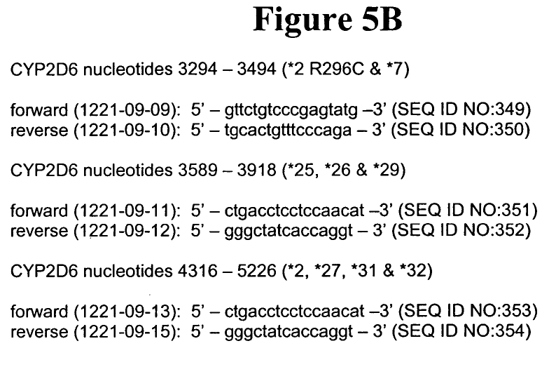

Ten target sequences were selected from a set of pre-validated SNP-containing sequences, available in a TWT in-house oligonucleotide order entry database. Each target contains a single nucleotide polymorphism (SNP) to which an INVADER assay had been previously designed. The INVADER assay oligonucleotides were designed by the INVADER CREATOR software (Third Wave Technologies, Inc. Madison, Wis.), thus the footprint region in this example is defined as the INVADER “footprint”, or the bases covered by the INVADER and the probe oligonucleotides, optimally positioned for the detection of the base of interest, in this case, a single nucleotide polymorphism (See FIG. 5). About 200 nucleotides of each of the 10 target sequences were analyzed for the amplification primer design analysis, with the SNP base residing about in the center of the sequence. The sequences are shown in FIG. 5.

Criteria of maximum and minimum probe length (defaults of 30 nucleotides and 12 nucleotides, respectively) were defined, as was a range for the probe melting temperature Tm of 50-60° C. In this example, to select a probe sequence that will perform optimally at a pre-selected reaction temperature, the melting temperature (Tₘ) of the oligonucleotide is calculated using the nearest-neighbor model and published parameters for DNA duplex formation (Allawi and SantaLucia, Biochemistry, 36:10581 [1997], herein incorporated by reference). Because the assay's salt concentrations are often different than the solution conditions in which the nearest-neighbor parameters were obtained (1M NaCl and no divalent metals), and because the presence and concentration of the enzyme influence optimal reaction temperature, an adjustment should be made to the calculated Tₘ to determine the optimal temperature at which to perform a reaction. One way of compensating for these factors is to vary the value provided for the salt concentration within the melting temperature calculations. This adjustment is termed a ‘salt correction’. The term “salt correction” refers to a variation made in the value provided for a salt concentration for the purpose of reflecting the effect on a Tₘ calculation for a nucleic acid duplex of a non-salt parameter or condition affecting said duplex. Variation of the values provided for the strand concentrations will also affect the outcome of these calculations. By using a value of 280 nM NaCl (SantaLucia, Proc Natl Acad Sci USA, 95:1460 [1998], herein incorporated by reference) and strand concentrations of about 10 pM of the probe and 1 fM target, the algorithm for used for calculating probe-target melting temperature has been adapted for use in predicting optimal primer design sequences.

Next, the sequence adjacent to the footprint region, both upstream and downstream were scanned and the first A or C was chosen for design start such that for primers described as 5′-N[x]-N[x-1]- . . . -N[4]-N[3]-N[2]-N[1]-3′, where N[1] should be an A or C. Primer complementarity was avoided by using the rule that: N[2]-N[1] of a given oligonucleotide primer should not be complementary to N[2]-N[1] of any other oligonucleotide, and N[3]-N[2]-N[1] should not be complementary to N[3]-N[2]-N[1] of any other oligonucleotide. If these criteria were not met at a given N[1], the next base in the 5′ direction for the forward primer or the next base in the 3′ direction for the reverse primer will be evaluated as an N[1] site. In the case of manual analysis, A/C rich regions were targeted in order to minimize the complementarity of 3′ ends.

In this example, an INVADER assay was performed following the multiplex amplification reaction. Therefore, a section of the secondary INVADER reaction oligonucleotide (the FRET oligonucleotide sequence, see FIG. 2) was also incorporated as criteria for primer design; the amplification primer sequence should be less than 80% homologous to the specified region of the FRET oligonucleotide.

The output primers for the 10-plex multiplex design are shown in FIG. 5). All primers were synthisized according to standard oligonucleotide chemistry, desalted (by standard methods) and quantified by absorbance at A260 and diluted to 50 μM concentrated stock. Multiplex PCR was then carried out using 10-plex PCR using equimolar amounts of primer (0.01 uM/primer) under the following conditions; 100 mMKCl, 3 mMMgCl, 10 mM Tris pH8.0, 200 uM dNTPs, 2.5 U taq, and 10 ng of human genomic DNA (hgDNA) template in a 50 ul reaction. The reaction was incubated for (94 C/30 sec, 50 C/44 sec.) for 30 cycles. After incubation, the multiplex PCR reaction was diluted 1:10 with water and subjected to INVADER analysis using INVADER Assay FRET Detection Plates, 96 well genomic biplex, 100 ng Cleavase VIII, INVADER assays were assembled as 15 ul reactions as follows; 1 ul of the 1:10 dilution of the PCR reaction, 3 ul of PPI mix, 5 ul of 22.5 mM MgCl2, 6 ul of dH20, covered with 15 ul of Chillout. Samples were denatured in the INVADER biplex by incubation at 95 C for 5 min., followed by incubation at 63 C and fluorescence measured on a Cytofluor 4000 at various timepoints.

Using the following criteria to accurately make genotyping calls (FOZ_FAM+FOZ_RED−2>0.6), only 2 of the 10 INVADER assay calls can be made after 10 minutes of incubation at 63 C, and only 5 of the 10 calls could be made following an additional 50 min of incubation at 63 C (60 min.). At the 60 min time point, the variation between the detectable FOZ values is over 100 fold between the strongest signal (41646, FAM_FOZ+RED_FOZ−2=54.2, which is also is far outside of the dynamic range of the reader) and the weakest signal (67356, FAM_FOZ+RED_FOZ−2=0.2). Using the same INVADER assays directly against 100 ng of human genomic DNA (where equimolar amounts of each target would be available), all reads could be made with in the dynamic range of the reader and variation in the FOZ values was approximately seven fold between the strongest (53530, FAM_FOZ+RED_FOZ−2=3.1) and weakest (53530, FAM_FOZ+RED_FOZ−2=0.43) of the assays. This suggests that the dramatic discrepancies in FOZ values seen between different amplicons in the same multiplex PCR reaction is a function of biased amplification, and not variability attributable to INVADER assay. Under these conditions, FOZ values generated by different INVADER assays are directly comparable to one another and can reliably be used as indicators of the efficiency of amplification.

Estimation of amplification factor of a given amplicon using FOZ values. In order to estimate the amplification factor (F) of a given amplicon, the FOZ values of the INVADER assay can be used to estimate amplicon abundance. The FOZ of a given amplicon with unknown concentration at a given time (FOZm) can be directly compared to the FOZ of a known amount of target (e.g. 100 ng of genomic DNA=30,000 copies of a single gene) at a defined point in time (FOZ₂₄₀, 240 min) and used to calculate the number of copies of the unknown amplicon. In equation 1, FOZm represents the sum of RED_FOZ and FAM_FOZ of an unknown concentration of target incubated in an INVADER assay for a given amount of time (m). FOZ₂₄₀ represents an empirically determined value of RED_FOZ (using INVADER assay 41646), using for a known number of copies of target (e.g. 100 ng of hgDNA≅30,000 copies) at 240 minutes.
F=((FOZₘ−1)*500/(FOZ₂₄₀−1))*(240/m)\hat{ }2 (equation 1a)

Although equation 1a is used to determine the linear relationship between primer concentration and amplification factor F, equation 1a′ is used in the calculation of the amplification factor F for the 10-plex PCR (both with equimolar amounts of primer and optimized concentrations of primer), with the value of D representing the dilution factor of the PCR reaction. In the case of a 1:3 dilution of the 50 ul multiplex PCR reaction. D=0.3333.
F=((FOZₘ−2)*500/(FOZ₂₄₀−1)*D)*(240/m)\hat{ }2 (equation 1a′)

Although equations 1a and 1a′ will be used in the description of the 10-plex multiplex PCR, a more correct adaptation of this equation was used in the optimization of primer concentrations in the 107 plex PCR. In this case, FOZ₂₄₀₌ the average of FAM_FOZ₂₄₀+RED_FOZ₂₄₀ over the entire INVADER MAP plate using hgDNA as target (FOZ₂₄₀₌3.42) and the dilution factor D is set to 0.125.
F=((FOZₘ−2)*500/(FOZ₂₄₀−2)*D)*(240/m)\hat{ }2 (equation 1b)

It should be noted that in order for the estimation of amplification factor F to be more accurate, FOZ values should be within the dynamic range of the instrument on which the reading are taken. In the case of the Cytofluor 4000 used in this study, the dynamic range was between about 1.5 and about 12 FOZ.

Section 3. Linear Relationship Between Amplification Factor and Primer Concentration.

In order to determine the relationship between primer concentration and amplification factor (F), four distinct uniplex PCR reactions were run at using primers 1117-70-17 and 1117-70-18 at concentrations of 0.01 uM, 0.012 uM, 0.014 uM, 0.020 uM respectively. The four independent PCR reactions were carried out under the following conditions; 100 mM KCl, 3 mM MgCl, 10 mM Tris pH 8.0, 200 uM dNTPs using 10 ng of hgDNA as template. Incubation was carried out at (94 C/30 sec., 50 C/20 sec.) for 30 cycles. Following PCR, reactions were diluted 1:10 with water and run under standard conditions using INVADER Assay FRET Detection Plates, 96 well genomic biplex, 100 ng CLEAVASE VIII enzyme. Each 15 ul reaction was set up as follows; 1 ul of 1:10 diluted PCR reaction, 3 ul of the PPI mix SNP#47932, 5 ul 22.5 mM MgCl2, 6 ul of water, 15 ul of Chillout. The entire plate was incubated at 95 C for 5 min, and then at 63 C for 60 min at which point a single read was taken on a Cytofluor 4000 fluorescent plate reader. For each of the four different primer concentrations (0.01 uM, 0.012 uM, 0.014 uM, 0.020 uM) the amplification factor F was calculated using equation 1a, with FOZm=the sum of FOZ_FAM and FOZ_RED at 60 minutes, m=60, and FOZ₂₄₀=1.7. In plotting the primer concentration of each reaction against the log of the amplification factor Log(F), a strong linear relationship was noted. Using the data points from the plotted primer concentration, the formula describing the linear relationship between amplification factor and primer concentration is described in equation 2:
Y=1.684X+2.6837 (equation 2a)

Using equation 2, the amplification factor of a given amplicon Log(F)=Y could be manipulated in a predictable fashion using a known concentration of primer (X). In a converse manner, amplification bias observed under conditions of equimolar primer concentrations in multiplex PCR, could be measured as the “apparent” primer concentration (X) based on the amplification factor F. In multiplex PCR, values of “apparent” primer concentration among different amplicons can be used to estimate the amount of primer of each amplicon required to equalize amplification of different loci:
X=(Y−2.6837)/1.68 (equation 2b)
Section 4.Calculation of Apparent Primer Concentrations from a Balanced Multiplex Mix.

As described in a previous section, primer concentration can directly influence the amplification factor of given amplicon. Under conditions of equimolar amounts of primers, FOZm readings can be used to calculate the “apparent” primer concentration of each amplicon using equation 2. Replacing Y in equation 2 with log(F) of a given amplification factor and solving for X, gives an “apparent” primer concentration based on the relative abundance of a given amplicon in a multiplex reaction. Using equation 2 to calculate the “apparent” primer concentration of all primers (provided in equimolar concentration) in a multiplex reaction (FIG. 3A), provides a means of normalizing primer sets against each other. In order to derive the relative amounts of each primer that should be added to an “Optimized” multiplex primer mix R, each of the “apparent” primer concentrations should be divided into the maximum apparent primer concentration (Xₘₐₓ), such that the strongest amplicon is set to a value of 1 and the remaining amplicons to values equal or greater than 1
R[n]=Xmax/X[n] (equation 3)

Using the values of R[n] as an arbitrary value of relative primer concentration, the values of R[n] are multipled by a constant primer concentration to provide working concentrations for each primer in a given multiplex reaction. In the example shown, the amplicon corresponding to SNP assay 41646 has an R[n] value equal to 1. All of the R[n] values were multipled by 0.01 uM (the original starting primer concentration in the equimolar multiplex pcr reaction) such that lowest primer concentration is R[n] of 41646 which is set to 1, or 0.01 uM. The remainder of the primer sets were also proportionally increased. The results of multiplex PCR with the “optimized” primer mix are described below.

Section 5. Using Optimized Primer Concentrations in Multiplex PCR, Variation in FOZ's Among 10 INVADER Assays are Greatly Reduced.

Multiplex PCR was carried out using 10-plex PCR using varying amounts of primer based on the volume (X[max] was SNP41646, setting 1x=0.01 uM/primer). Multiplex PCR was carried out under conditions identical to those used in with equimolar primer mix; 100 mMKCl, 3 mMMgCl, 10 mM Tris pH8.0, 200 uM dNTPs, 2.5 U taq, and 10 ng of hgDNA template in a 50 ul reaction. The reaction was incubated for (94 C/30 sec, 50 C/44 sec.) for 30 cycles. After incubation, the multiplex PCR reaction was diluted 1:10 with water and subjected to INVADER analysis. Using INVADER Assay FRET Detection Plates, (96 well genomic biplex, 100 ng CLEAVSE VIII enzyme), reactions were assembled as 15 ul reactions as follows; 1 ul of the 1:10 dilution of the PCR reaction, 3 ul of the appropriate PPI mix, 5 ul of 22.5 mM MgCl2, 6 ul of dH20. An additional 15 ul of CHILL OUT was added to each well, followed by incubation at 95 C for 5 min. Plates were incubated at 63 C and fluorescence measured on a Cytofluor 4000 at 10 min.

Using the following criteria to accurately make genotyping calls (FOZ_FAM+FOZ_RED−2>0.6), all 10 of 10 (100%) INVADER calls can be made after 10 minutes of incubation at 63 C. In addition, the values of FAM+RED−2 (an indicator of overall signal generation, directly related to amplification factor (see equation 2)) varied by less than seven fold between the lowest signal (67325, FAM+RED−2=0.7) and the highest (47892, FAM+RED−2=4.3).

### Example 2

### Design of 101-plex PCR using the Software Application

Using the TWT Oligo Order Entry Database, 144 sequences of less than 200 nucleotides in length were obtained with SNP annotated using brackets to indicate the SNP position for each sequence (e.g. NNNNNNN[N_{(wt)}/N₍ₘₜ₎]NNNNNNNN). In order to expand sequence data flanking the SNP of interest, sequences were expanded to approximately 1 kB in length (500 nts flanking each side of the SNP) using BLAST analysis. Of the 144 starting sequences, 16 could not expanded by BLAST, resulting in a final set of 128 sequences expanded to approximately 1 kB length. These expanded sequences were provided to the user in Excel format with the following information for each sequence; (1) TWT Number, (2) Short Name Identifier, and (3) sequence. The Excel file was converted to a comma delimited format and used as the input file for Primer Designer INVADER CREATOR v1.3.3. software (this version of the program does not screen for FRET reactivity of the primers, nor does it allow the user to specify the maximum length of the primer). INVADER CREATOR Primer Designer v1.3.3., was run using default conditions (e.g. minimum primer size of 12, maximum of 30), with the exception of Tm_{low} which was set to 60 C. The output file contained 128 primer sets (256 primers), four of which were thrown out due to excessively long primer sequences (SNP # 47854, 47889, 54874, 67396), leaving 124 primers sets (248 primers) available for synthesis. The remaining primers were synthesized using standard procedures at the 200 nmol scale and purified by desalting. After synthesis failures, 107 primer sets were available for assembly of an equimolar 107-plex primer mix (214 primers). Of the 107 primer sets available for amplification, only 101 were present on the INVADER MAP plate to evaluate amplification factor.

Multiplex PCR was carried out using 101-plex PCR using equimolar amounts of primer (0.025 uM/primer) under the following conditions; 100 mMKCl, 3 mM MgCl, 10 mM Tris pH8.0, 200 uM dNTPs, and 10 ng of human genomic DNA (hgDNA) template in a 50 ul reaction. After denaturation at 95 C for 10 min, 2.5 units of Taq was added and the reaction incubated for (94 C/30 sec, 50 C/44 sec.) for 50 cycles. After incubation, the multiplex PCR reaction was diluted 1:24 with water and subjected to INVADER assay analysis using INVADER MAP detection platform. Each INVADER MAP assay was run as a 6 ul reaction as follows; 3 ul of the 1:24 dilution of the PCR reaction (total dilution 1:8 equaling D=0.125), 3 ul of 15 mM MgCl2 covered with covered with 6 ul of CHILLOUT. Samples were denatured in the INVADER MAP plate by incubation at 95 C for 5 min., followed by incubation at 63 C and fluorescence measured on a Cytofluor 4000 (384 well reader) at various timepoints over 160 minutes. Analysis of the FOZ values calculated at 10, 20, 40, 80, 160 min. shows that correct calls (compared to genomic calls of the same DNA sample) could be made for 94 of the 101 amplicons detectable by the INVADER MAP platform. This provides proof that the INVADER CREATOR Primer Designer software can create primer sets which function in highly multiplex PCR.

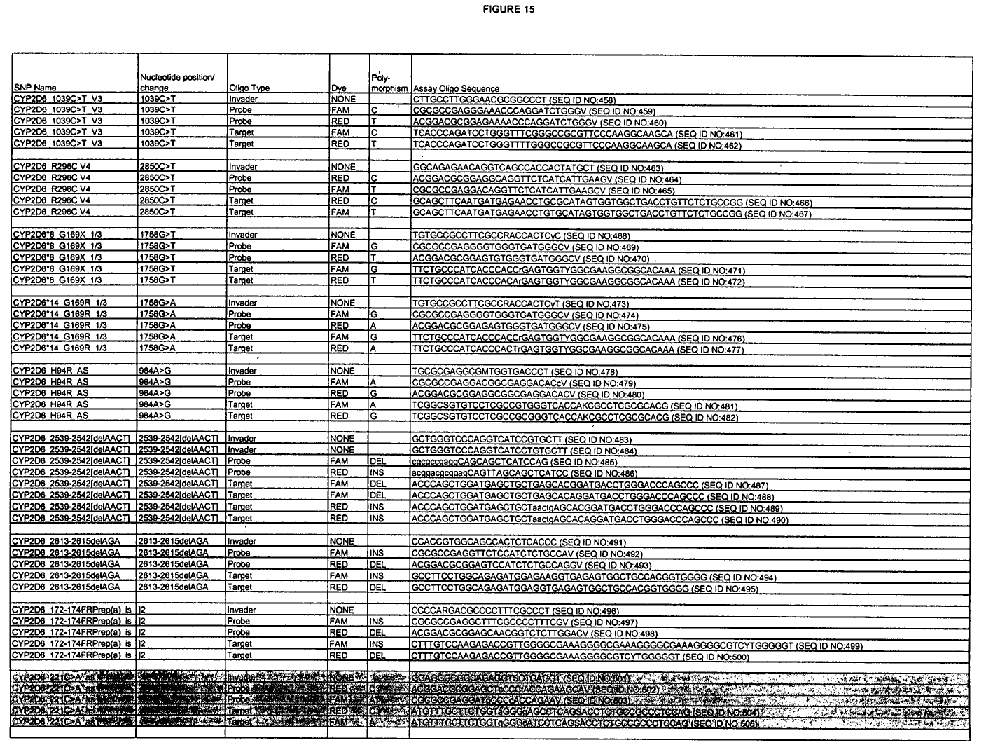

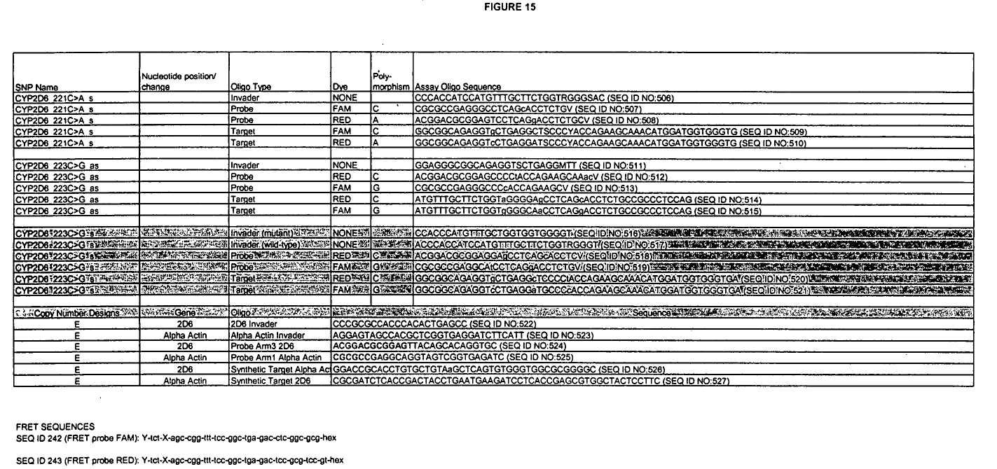

In using the FOZ values obtained throughout the 160 min. time course, amplification factor F and R[n] were calculated for each of the 101 amplicons. R[nmax] was set at 1.6, which although Low end corrections were made for amplicons which failed to provide sufficient FOZm signal at 160 min., assigning an arbitrary value of 12 for R[n]. High end corrections for amplicons whose FOZm values at the 10 min. read, an R[n] value of 1 was arbitrarily assigned. Optimized primer concentrations of the 101-plex were calculated using the basic principles outlined in the 10-plex example and equation 1b, with an R[n] of 1 corresponding to 0.025 uM primer (see FIG. 15 for various primer concentrations). Multiplex PCR was under the following conditions; 100 mMKCl, 3 mM MgCl, 10 mM Tris pH8.0, 200 uM dNTPs, and 10 ng of human genomic DNA (hgDNA) template in a 50 ul reaction. After denaturation at 95 C for 10 min, 2.5 units of Taq was added and the reaction incubated for (94 C/30 sec, 50 C/44 sec.) for 50 cycles. After incubation, the multiplex PCR reaction was diluted 1:24 with water and subjected to INVADER analysis using INVADER MAP detection platform. Each INVADER MAP assay was run as a 6 ul reaction as follows; 3 ul of the 1:24 dilution of the PCR reaction (total dilution 1:8 equaling D=0.125), 3 ul of 15 mM MgCl2 covered with covered with 6 ul of CHILLOUT. Samples were denatured in the INVADER MAP plate by incubation at 95 C for 5 min., followed by incubation at 63 C and fluorescence measured on a Cytofluor 4000 (384 well reader) at various timepoints over 160 minutes. Analysis of the FOZ values was carried out at 10, 20, and 40 min. and compared to calls made directly against the genomic DNA. A comparison was made between calls made at 10 min. with a 101-plex PCR with the equimolar primer concentrations versus calls that were made at 10 min. with a 101-plex PCR run under optimized primer concentrations. Under equimolar primer concentration, multiplex PCR results in only 50 correct calls at the 10 min time point, where under optimized primer concentrations multiplex PCR results in 71 correct calls, resulting in a gain of 21 (42%) new calls. Although all 101 calls could not be made at the 10 min timepoint, 94 calls could be made at the 40 min. timepoint suggesting the amplification efficency of the majority of amplicons had improved. Unlike the 10-plex optimization that only required a single round of optimization, multiple rounds of optimization may be required for more complex multiplexing reactions to balance the amplification of all loci.

### Example 3

### Characterization of Cytochrome p450 2D6 Alleles Using Triplex PCR and the INVADER Assay System

The field of pharmacogenetics is advancing rapidly as increasing numbers of functional polymorphisms in proteins essential for drug action are identified. One of the most clinically important of these proteins is an enzyme in the cytochrome P450 family, debrisoquine 4-hydroxylase, or cytochrome P450 2D6 (CYP2D6), the gene for which is found on chromosome band 22q13.1. This enzyme metabolizes about 25% of all therapeutic drugs, including beta-blockers, serotonin reuptake inhibitors, anti-emetics, tricyclic anti-depressants, anti-arrhythmics, and nicotine. In addition, CYP2D6 metabolizes many environmental xenobiotic substances. Hence, the metabolic status of the enzyme has been linked to a wide range of illnesses such as liver cancer (Agundez, J. A., et al. Lancet, 1995. 345(8953):830) and Parkinson's disease (Smith, C. A., et al. Lancet, 1992. 339(8806):1375).

Currently, more than 70 polymorphisms have been identified within the exonic and promoter regions of CYP2D6; an equal number of haplotypes (e.g., see the world wide web site at imm.ki.se/CYPalleles/cyp2d6.html) have also been identified. Numerous genetic variations (more than 20 polymorphisms, a gene deletion, and a number of gene conversion events) cause decreased CYP2D6 activity. Depending on ethno-geographic origins, the overall incidence of poor metabolizer (PM) status in the general population ranges between 1-8%, (Sachse, C., et al., Am J Hum Genet, 1997. 60(2):284). In addition, multiple copies of alleles of CYP2D6 have been associated with extensive (EM) and ultra-rapid (UM) metabolizers (Johansson, I., et al., Proc Natl Acad Sci USA, 1993. 90(24): p. 11825; Lovlie, R., et al., FEBS Lett, 1996. 392(1):30).

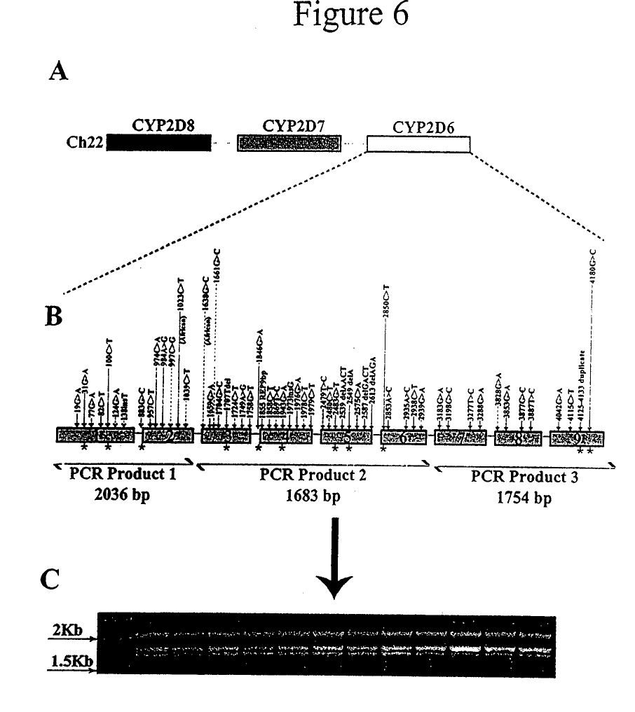

Directly upstream of the approximately 5-kb CYP2D6 gene lay two CYP2D6 pseudogenes, CYP2D7 and CYP2D8 (FIG. 6A). Both pseudogenes are highly homologous (97% and 92%, respectively) to the exonic sequence of the CYP2D6 gene (Kimura, S., et al., Am J Hum Genet, 1989. 45(6):889). Many rare alleles found in CYP2D6 also occur in CYP2D7 and CYP2D8. Despite the importance of the CYP2D6 enzyme in drug metabolism and adverse drug effects, the complexity in its genomic region has hampered attempts to develop clinical genetic tests for variations in this enzyme. Described here is a simple, scalable, and comprehensive CYP2D6 genotyping strategy that, in some embodiments, combines selective amplification of the CYP2D6 gene with the specificity of the invasive signal amplification reaction, or INVADER reaction, or similar technologies.

PCR-INVADER Assay Strategy

As described above, the CYP2D6 genomic region contains two adjacent pseudogenes, CYP2D7 and CYP2D8. To prevent false positive results or inflated wild type signals caused by INVADER oligonucleotides hybridizing to the pseudogenes, a series of PCR primers that specifically allow amplification of only CYP2D6 was devised. This PCR product was then used as a target for the NVADER assay reaction.

The INVADER Assay Reaction.

The biplex format of the INVADER DNA assay enables simultaneous detection of two DNA sequences in a single well, such as two variants of a particular polymorphism. The biplex format uses two different allele-specific primary probes, each with a unique 5′ flap, and two different FRET cassettes, each with a spectrally distinct fluorophore. By design, the released 5′-flaps will bind only to their respective FRET cassettes to generate a target-specific signal.

CYP2D6-Specific Triplex PCR for Genotyping

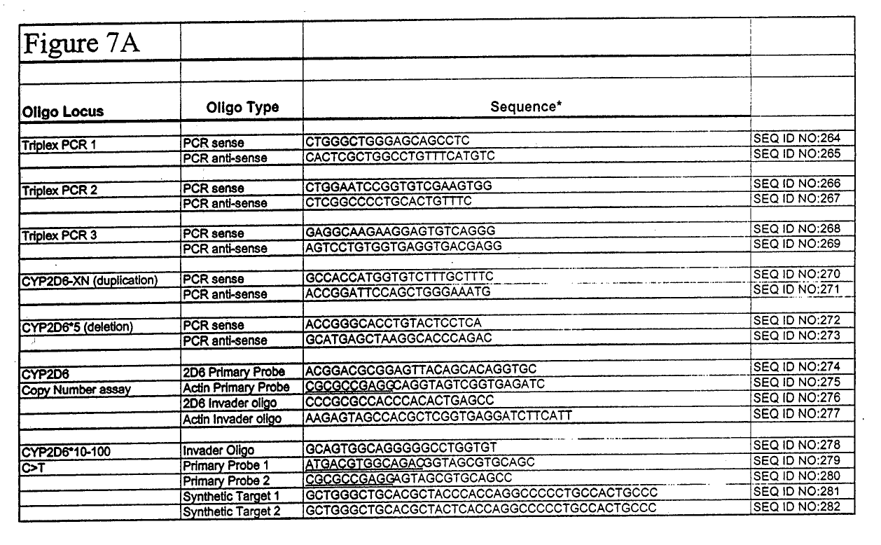

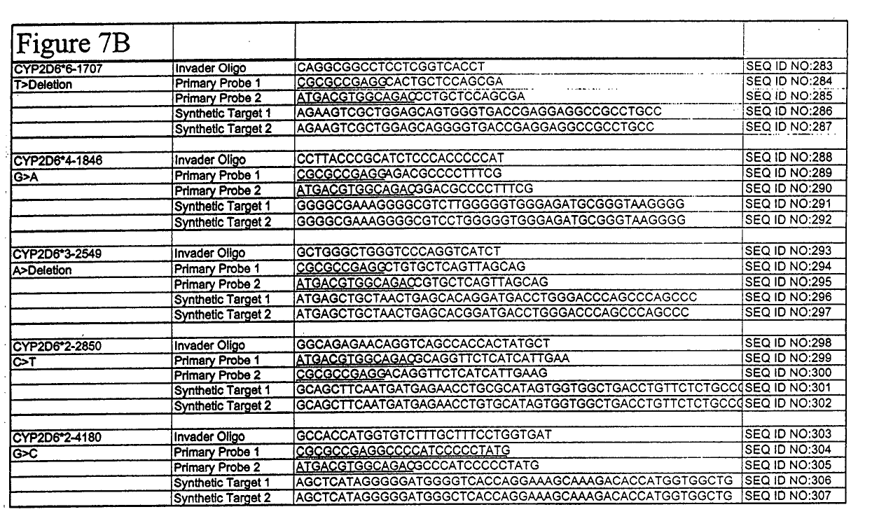

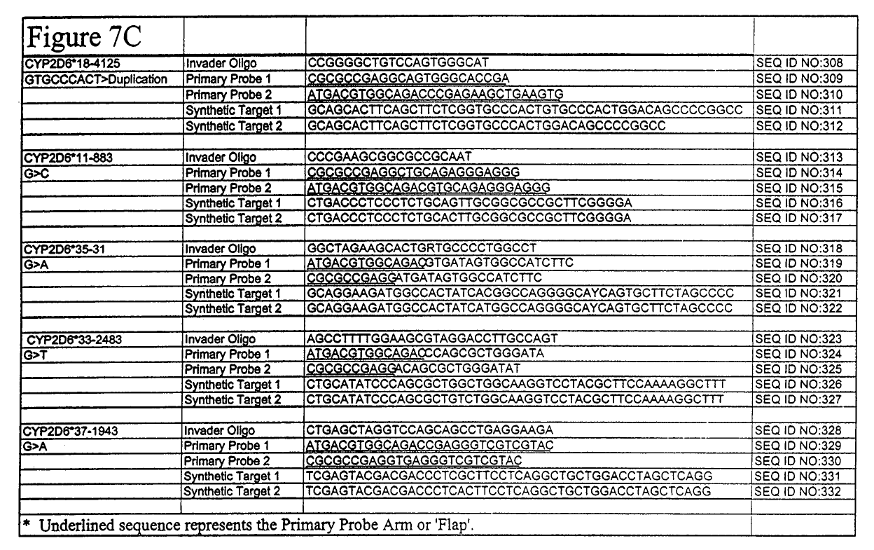

The CYP2D6 region encompasses approximately 5 kb of genomic sequence. While this is well within the capabilities of long-range PCR technologies, it depends heavily on template quality. With this in mind, to improve the robustness of the PCR reaction, the CYP2D6 genomic region was divided into three shorter and non-overlapping PCR fragments to pool together in a single triplex PCR reaction. All primers were designed over CYP2D6-specific sequence within 5′-, 3′- or intronic regions and to have a melting temperature of 68° C. (FIG. 7 contains the primer sequences used). Primer pair 1 amplifies exons 1 and 2 generating a 2036-bp product, primer pair 2 amplifies exons 3 to 6 generating a 1683-bp product and primer pair 3 amplifies exons 7 to 9 generating a 1754-bp product.

DNA from a group of 181 anonymous donors was used in this set of experiments. The DNA was isolated using the Qiagen QIAmp whole blood kit (Qiagen, Valencia, Calif.). The CYP2D6-specific triplex PCR reactions were performed using the ‘Herculase Hotstart’ PCR system (Stratagene, La Jolla, Calif. Cat. No. 600310) with 10-200 ng of genomic DNA, 250 μM dNTPs, 0.4 μM of each primer, 2% DMSO and 2.5 units of the enzyme supplied in a final volume of 50 μL. The reaction was incubated on a ThermoHybaid PCR express Thermocycler (ThermoHybaid, Franklin, Mass.) using the following cycling parameters: 95° C. for 5 minutes, followed by 35 cycles of 95° C. for 30 seconds and 68° C. for 4 minutes, and finishing with a 10-minute extension cycle at 68° C. For verification purposes, 10 μl of the PCR product was initially visualised on a 1% agarose gel containing ethidium bromide. FIG. 6C provides an example of the three PCR products generated in this step.

INVADER CYP2D6 Genotyping Assays

INVADER assays were designed for the following CYP2D6 polymorphisms: CYP2D6*2-2850C to T; *2-4180G to C; *3-2549A Del; *4-1846G to A; *6-1707T Del; *10-100C to T; *11-883G to C; *18-4125GTGCCCACT Duplication; *33-2483G to T; *35-31G to A and *37-1943G to A. The number after the * represents the CYP2D6 haplotype and the number after the hyphen represents the position of the polymorphism in relation to the translational start codon (Daly, A. K., et al., Pharmacogenetics, 1996. 6(3): p. 193). FIG. 6B indicates the relative positions of these 11 assays in the CYP2D6 genomic region. Each assay was designed for non-synonymous polymorphisms. The CYP2D6*2, *10, *33 and *35 haplotypes are among the most common functional alleles in Caucasians aside from CYP2D6* 1, and CYP2D6*3; *4 and *6 are among the most common non-functional alleles in Caucasians apart from the deletion allele CYP2D6*5 (Gaedigk, A., et al., Pharmacogenetics, 1999. 9(6):669; Marez, D., et al, Pharmacogenetics, 1997. 7(3):193). Each PCR product is was detected by at least two INVADER assays. The Table in FIG. 7 provides the sequences for the INVADER and probe oligonucleotides for each assay. Each assay used a synthetic oligonucleotide complementary to both the INVADER and probe oligonucleotides as a positive control. The INVADER reactions were performed using 384-well INVADER Assay FRET detection plates, which contain CLEAVASE enzyme, F dye (F=fluorescein) and R dye (R=REDMOND RED) FRET cassettes, and reaction buffer, dried down in each well. REDMOND RED is from Epoch Biosciences, Bothell, Wash. Cassettes are shown in the Cassette Table, below.

Briefly, 3 μl of a 1/20 dilution of the CYP2D6-specific PCR products or a negative control (T10e0.1 buffer (10 mM Tris, pH 8, 0.1 mM EDTA)) were added to the appropriate wells followed by addition of 3 μl of the appropriate primary probes/INVADER oligonucleotide/MgCl₂ mix. After the additions, each reaction was overlayed with 7 μl of molecular biology-grade mineral oil to prevent evaporation (Sigma-Aldrich, Steinheim, Germany). Each 6-μl reaction contained 10 ng CLEAVASE enzyme, 4% PEG 8000, 2% glycerol, 0.06% NP 40, 0.06% Tween 20, 12 ug/ml BSA, 0.58 μM each of F dye and R dye FRET cassettes, 7 mM MgCl2, 0.7 μM of each allele-specific primary probe, and 0.07 μM INVADER oligonucleotide. Following reagent dispensing, plates were spun for 10 seconds at 1,000 rpm, then incubated at 95° C. for 5 minutes and then 63° C. for 30 minutes using ThermoHybaid PCR Express Thermocycler. Fluorescence was measured directly at the end of the incubation period using a CytoFluor 4000 fluorescence plate reader (Applied Biosystems, Foster City, Calif.). The settings used were 485/20 nm excitation/bandwidth and 530/25 nm emission/bandwidth for F dye detection and 560/20 nm excitation/bandwidth and 620/40 nm emission/bandwidth for R dye detection.

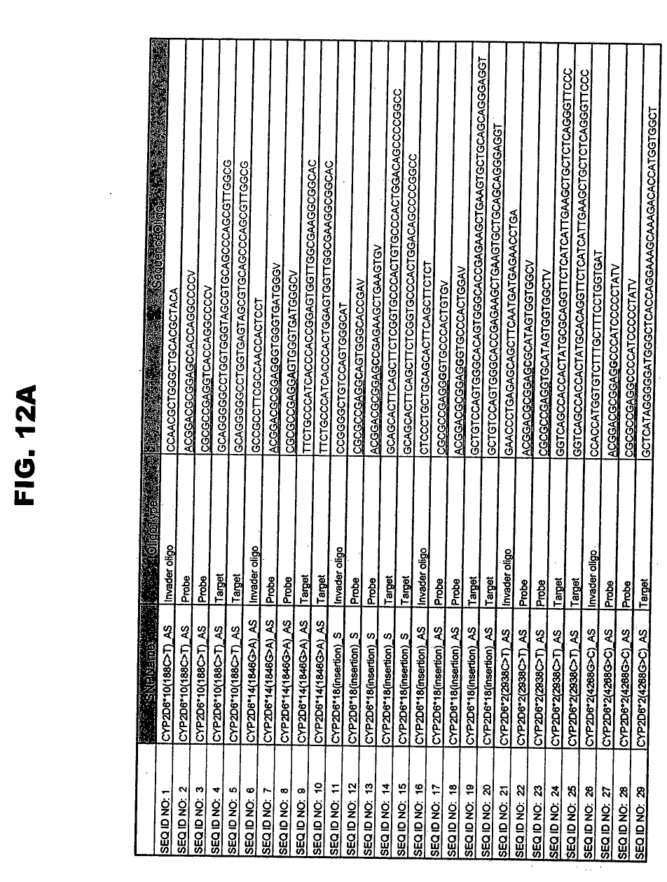

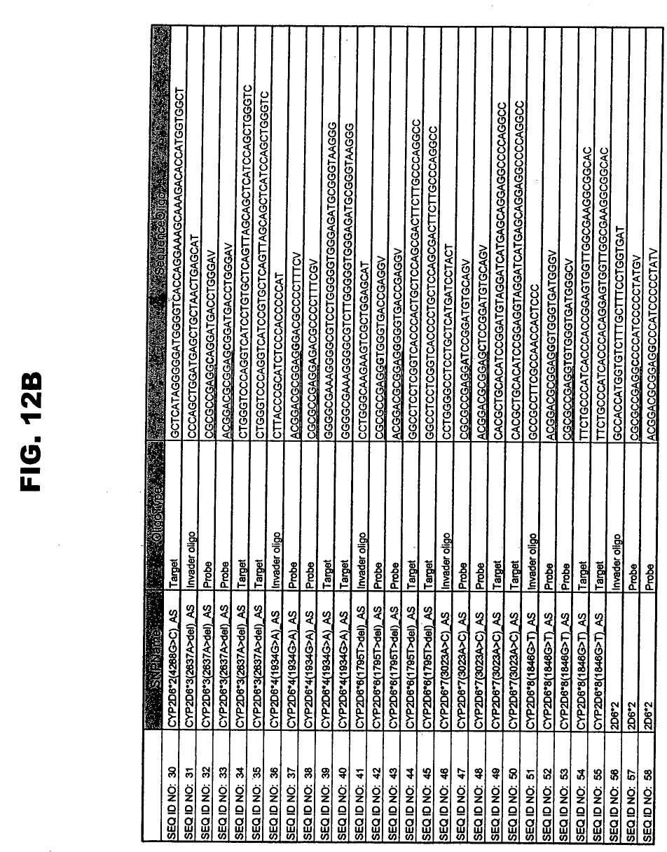

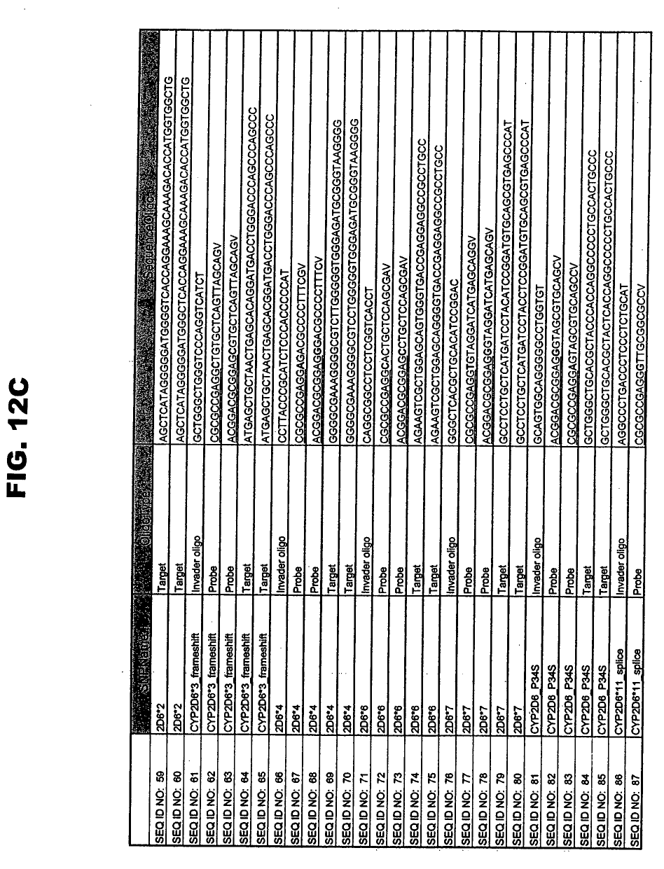

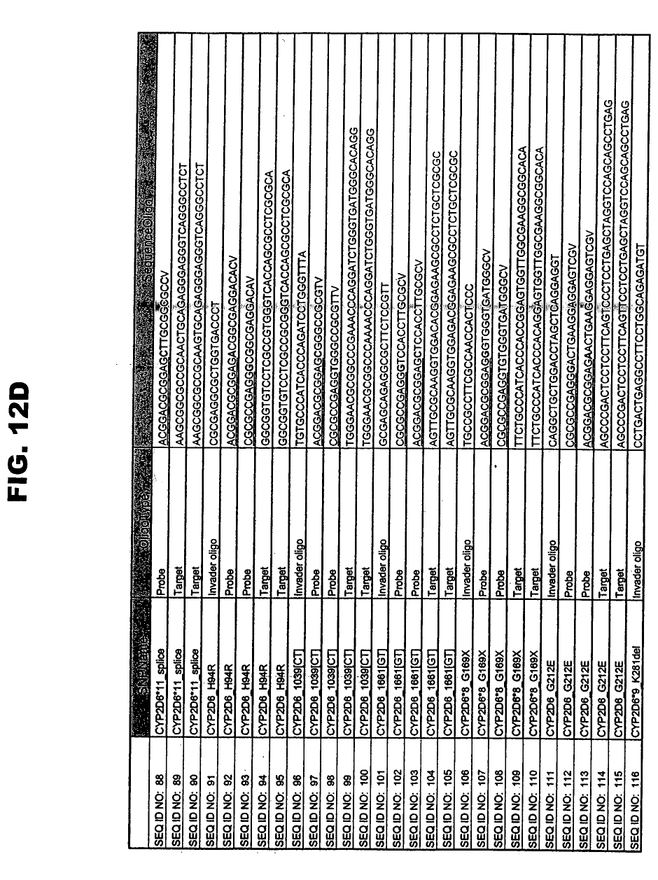

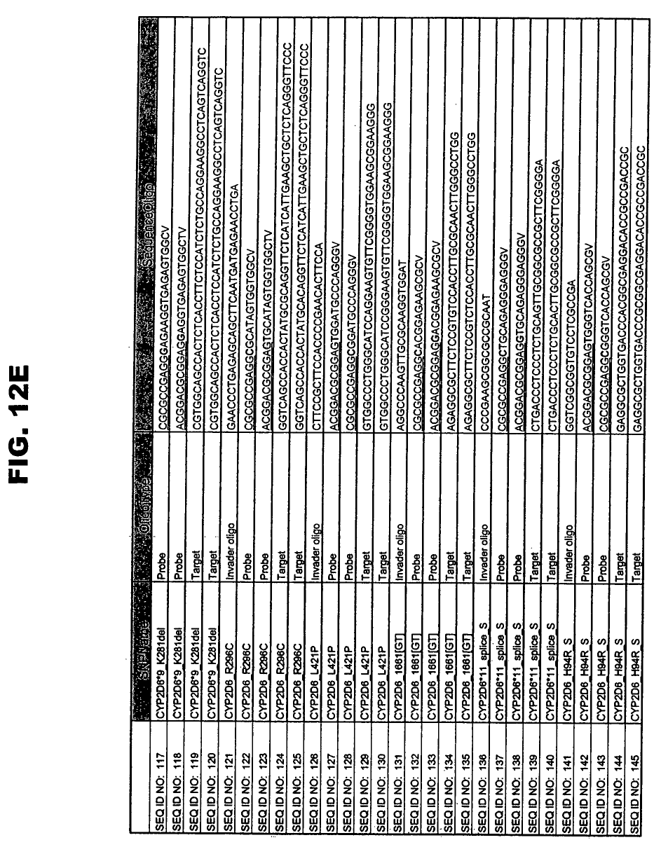

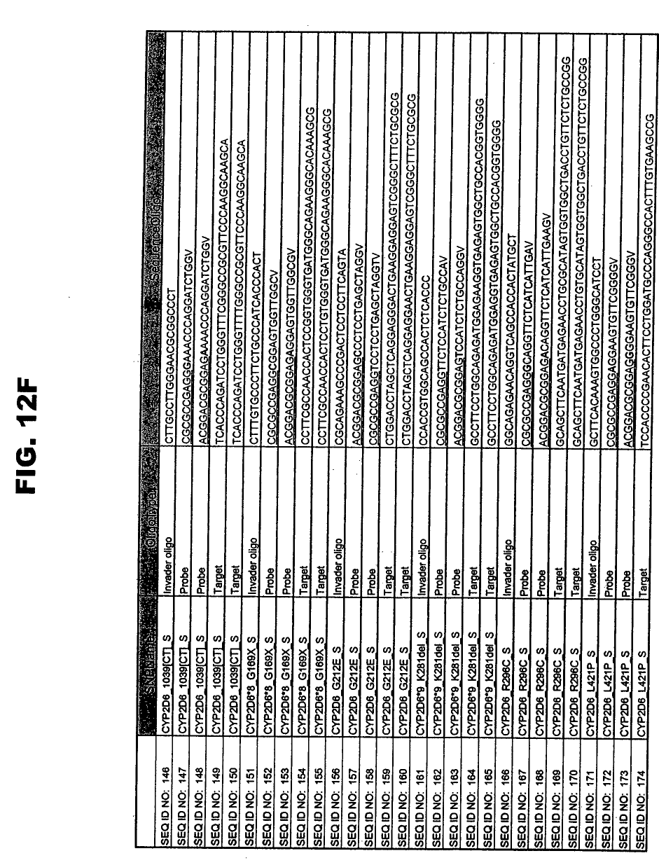

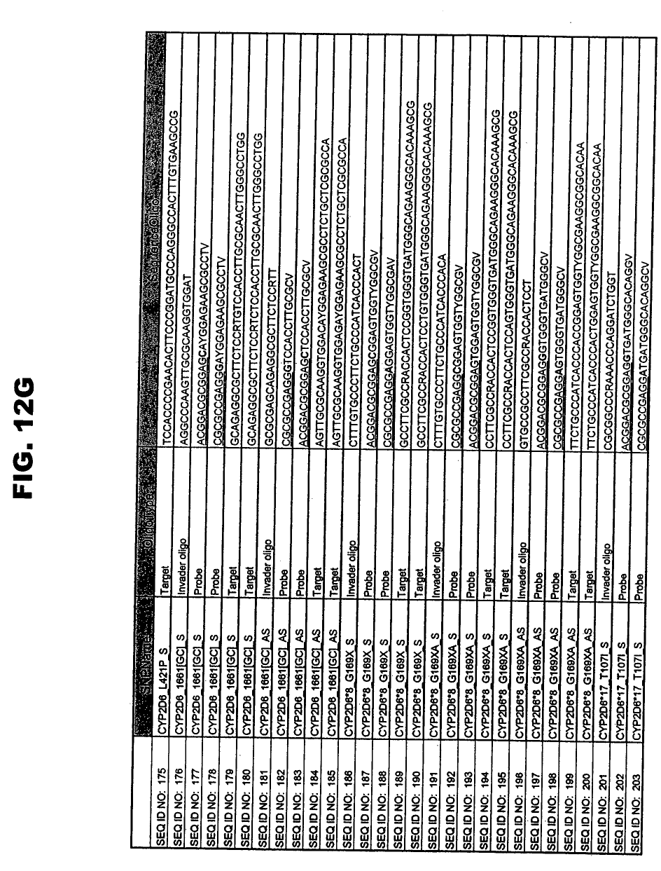

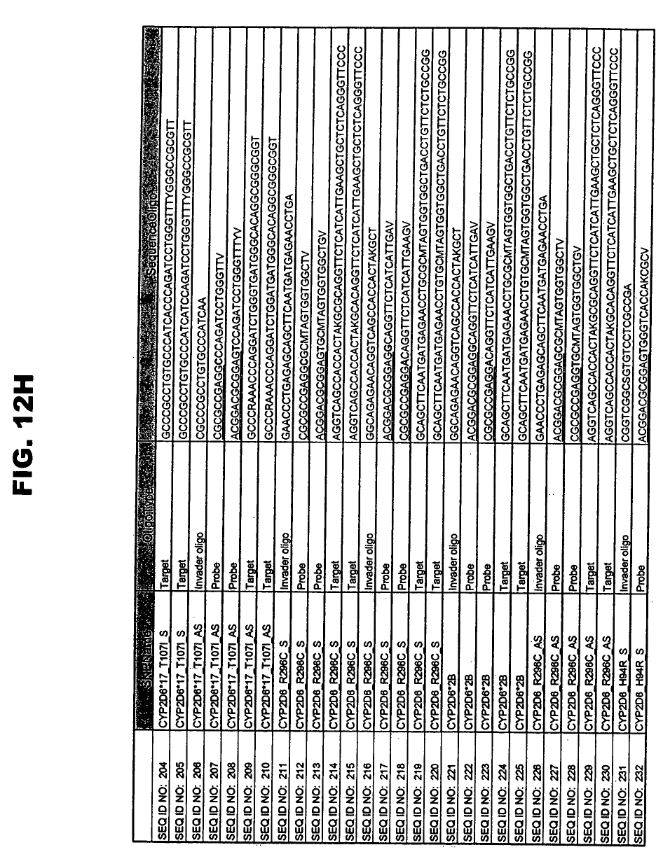

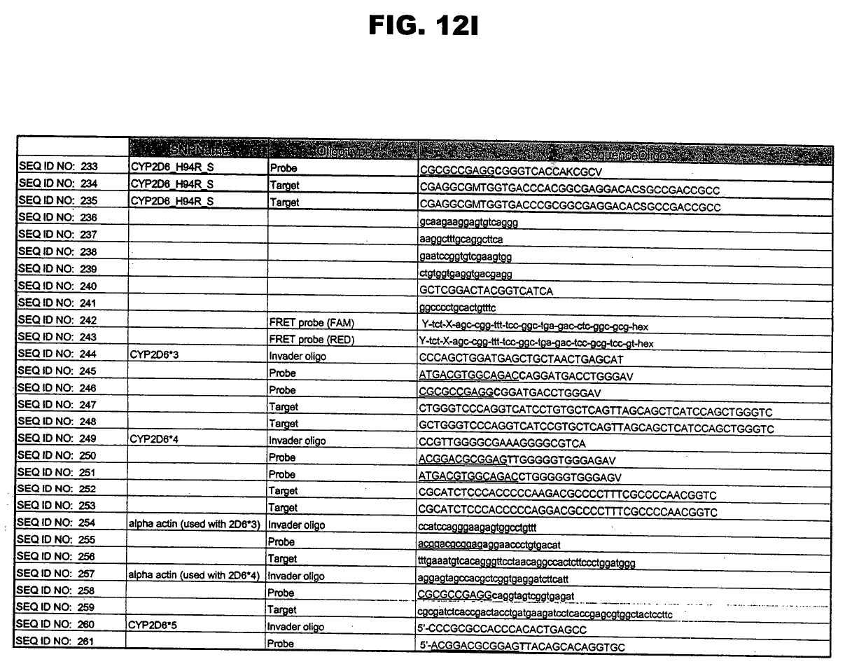

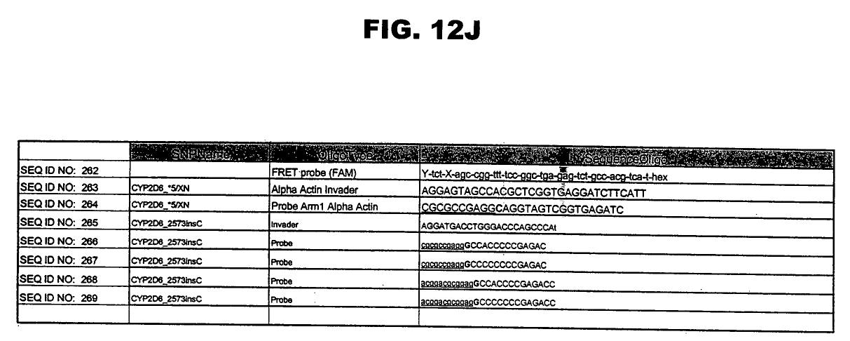

In addition to assays to detect the CYP2D6 variants described in FIG. 7, further designs were created to detect the alleles listed in FIG. 12. In this figure, the underlined bases in the probe oligos indicate the 5′ flaps removed by the CLEAVASE enzyme, and the bold base, the polymorphic position. V at the 3′ end of probe sequences refers to a hexanediol blocker.

Modified triplex PCR conditions were also developed as follows. All primers were designed to amplify CYP2D6-specific sequence within 5′-, 3′- or intronic regions. Three primer sets were chosen to have a melting temperature of 55° C. The CYP2D6-specific triplex PCR reactions were performed using the ‘Herculase Hotstart’ PCR system (Stratagene, La Jolla, Calif. Cat. No. 600310) with 10-200 ng of genomic DNA, 250 μM dNTPs, 0.4 μM of each primer, 2% DMSO and 2.5 units of the enzyme supplied in a final volume of 50 μL. The following cycling parameters were used: 95° C. for 10 minutes, followed by 35 cycles of 94° C. for 30 seconds, 55° C. for 1 minute, and 72° C. for 3 minutes, and finishing with a 10-minute extension cycle at 72° C. For verification purposes, 10 μl of the PCR product was initially visualised on a 1% agarose gel containing ethidium bromide. The primers are included in FIG. 12 as SEQ ID NOs: 236-241.

The INVADER assays to detect these variants were as described above except that the CLEAVASE enzyme used was the CLEAVASE XI enzyme and the FRET cassettes used were SEQ ID NOs: 242-243 (FIG. 12). In all cases, sequences for synthetic targets are listed in FIG. 12; these oligonucleotides may be used with the appropriate INVADER and probe oligos in positive INVADER assay control experiments using standard reaction conditions.

Analysis of CYP2D6*3 and *4 Alleles Directly from Genomic DNA

Direct detection of CYP2D6 variants from genomic DNA is complicated by the presence of pseudogene sequences, e.g. CYP2D7 and 8. While methods that amplify discrete genomic regions, such as PCR, can be useful to separate such a region of interest from its genomic context, in some instances it is desirable to avoid the use of target amplification methods. An approach to detecting variants of CYP2D6 via direct analysis of genomic DNA was developed using the INVADER assay and was based on the detection of heterologous internal control sequences in lieu of the wild-type CYP2D6 allele. Biplexed INVADER assays were designed to detect mutant alleles of the CYP2D6 gene and a conserved sequence in the α-actin gene as an internal control.

Standard INVADER reactions were set up using a 96-well microtiter plate dry-down format as described previously using 7.5 μl of denatured genomic DNA, or 10 ng/μl of tRNA in distilled water and the CLEAVASE XI enzyme. The FRET probes used were SEQ ID NOs: 242 (FAM) and 243 (RED). Experiments to detect the CYP2D6*3 variant included 7 μM primary probe (SEQ ID NO: 246) and 0.7 μM INVADER oligonucleotide (SEQ ID NO: 244) and 5 μM α-actin primary probe (SEQ ID NO: 255) and 0.5 μM α-actin INVADER oligonucleotide (SEQ ID NO: 254 ). Experiments to detect the CYP2D6*4 allele included 7 μM primary probe (SEQ ID NO: 250) and 0.7 μM INVADER oligonucleotide (SEQ ID NO: 249) and 5 μM α-actin primary probe (SEQ ID NO: 258) and 0.5 μM α-actin INVADER oligonucleotide (SEQ ID NO: 257).

Cutoff values were set such that ratios of Net FOZ>0.15 indicated the presence of the mutant allele, either in a heterozygote or homozygous mutant. Given these cutoff values, 2 of 41 samples were determined to comprise the mutant allele and the remainder were wild-type. Validation of such genotype determinations can be accomplished by any of several approaches, including the PCR-INVADER assay method described previously in this example. Probe sequences (SEQ ID NOs: 245 for CYP2D6*3 and 251 for CYP2D6*4) that may be used in combination with the appropriate INVADER oligos for confirming the presence of the wild-type alleles in amplified fragments are listed in FIG. 12, including the appropriate FRET probe (SEQ ID NO: 262). As described above, probe and synthetic target sequences for both the variant and wild-type alleles are included and may be used in appropriate control and test reactions.

CYP2D6 INVADER Copy Number Assay

The INVADER system is directly quantitative and can be used to identify gene copy number by comparing the target gene signal (CYP2D6) with that of a reference gene that is known to be non-polymorphic for either duplication or deletion, such as the α-actin gene. Therefore, by using the relative ratios of the CYP2D6 and reference gene signals from each assay (similar to the way that ratios of the wild-type and variant signals are used to score a genotype, as described below), the deletion and duplication alleles of CYP2D6 can be identified and quantitated.

With the approval of the University of Wisconsin—Madison Institutional Review Board, the CYP2D6 copy number was assayed in 205 patients presenting for surgery at the University of Wisconsin Hospitals. Genomic DNA was isolated from whole blood using the PUREGENE DNA Isolation Kit (Gentra Systems, Minneapolis, Minn.) according to manufacturer's directions. INVADER assay detection of the CYP2D6 copy number was performed in duplicate using 96-well dry-down plates. In brief, 7 μl of pre-denatured DNA samples (15-20 ng/μl) or negative control (10 ng/μl solution of tRNA in T10e0.1 buffer (10 mM Tris, pH 8, 0.1 mM EDTA)) were added to the appropriate wells followed by addition of 8 μl of the appropriate primary probes/INVADER oligonucleotide/MgCl2 mix and then overlayed with 15 μl of molecular biology grade mineral oil (Sigma-Aldrich, Steinheim, Germany). Each 15-μl reaction contained 100 ng CLEAVASE enzyme, 4% PEG 8000, 2% glycerol, 0.06% NP 40, 0.06% Tween 20, 12 ug/ml BSA, 0.35 μM of each F dye and R dye FRET cassettes, 7.5 mM MgCl2, 0.7 μM of each allele-specific primary probe, and 0.07 μM INVADER oligonucleotide. Following the reagent dispensing, plates were spun for 10 seconds at 1,000 rpm, incubated at 63° C. for 4 hours in a PTC 100 thermocycler (MJResearch, Incline Village, Nev.) and then directly read in a Cytofluor 4000 fluorescence plate reader (Applied Biosystems, Foster City, Calif.) using the same settings given above.

Assignments based on INVADER assay results were confirmed by long-range PCR. If CYP2D6 is deleted (CYP2D6*5), then a 3.5-kb PCR product will result (Steen, V. M., et al., Pharmacogenetics, 1995. 5(4):215). If there are duplicated or multiple copy CYP2D6 alleles then a 10-kb PCR product will result. Samples identified by the INVADER assay as either containing one or three copies of the CYP2D6 allele were subjected to PCR. Both the gene deletion and duplication PCR assays were performed with the GeneAmp XL PCR kit (Perkin Elmer, Foster City, Calif., Cat. No. N808-0192). The deletion primers (FIG. 7) were used in a 50-μl PCR reaction with 200 ng DNA, 1×XL reaction buffer, 1.1 mM Mg(OAc)₂, 200 mM of each dNTP, 0.3 mM of each primer, and 1 unit of DNA polymerase. The cycling parameters used were: 94° C. for 1 minute followed by 35 cycles of 94° C. for 1 minute, 65° C. for 30 seconds and 68° C. for 5 minutes, and then finishing with a 12-minute 72° C. extension cycle. The resulting 3.5-kb PCR products were detected by gel electrophoresis on a 1% agarose gel containing ethidium bromide. The duplication PCR primers amplified a fragment between exon 9 of the proximal CYP2D6 copy and intron 2 of a distal CYP2D6 copy in regions specific to CYP2D6 (Johansson, I., et al., Pharmacogenetics, 1996. 6(4):351; FIG. 7). The 50-μl PCR reaction contained 400 ng DNA, 1×XL reaction buffer, 1.0 mM Mg(OAc)₂, 200 mM of each dNTP, 0.3 mM of each primer, and 3 units of DNA polymerase. The cycling parameters used were: 94° C. for 1 minute followed by 35 cycles of 94° C. for 1 minute, 61.4° C. for 30 seconds, and 68° C. for 10 minutes finishing with a 12-minute 72° C. extension cycle. The resulting 10-kb PCR products were detected by gel electrophoresis on a 1% agarose gel containing ethidium bromide. We observed no amplification in alleles lacking the duplication. However, conventional PCR could not determine the number of CYP2D6 duplications.

Data Analysis for Genotype and Copy Number Determination

Data were exported into the Microsoft Excel program (Microsoft, Redmond, Wash.). For each allele of a given polymorphism, the Net Fold Over Zero (FOZ-1) values are calculated as follows:

$$\begin{matrix}Net F dye FOZ = \frac{F dye raw counts from sample}{F dye raw counts from negative control} - 1 \\ Net R dye FOZ = \frac{R dye raw counts from sample}{R dye raw counts from negative control} - 1\end{matrix}$$

Determination of the genotype or copy number was based on the ratio of the Net R dye FOZ value to the Net F dye FOZ value as shown below:

$$Allelic Ratio = \frac{Net R dye FOZ}{Net F dye FOZ}$$

In cases where the Net FOZ value was equal to or less than zero, the Net FOZ value was adjusted to 0.01 to avoid the generation of negative values or division by zero. An allelic ratio of equal to or less than 0.25 was scored as homozygous for the F dye allele, a ratio greater than 4 was scored as homozygous for the R dye allele and a ratio greater than 0.25 but less than 4 was scored as heterozygous. Values that fell within two ranges (greater than 0.25 and equal to or less than 0.4, or equal to or greater than 2.5 and equal to or less than 4) were designated as equivocal and the sample result was not included. Instances in which both the F dye and R dye Net FOZ values were less than 2, were recorded as low-signal. The allelic ratio was calculated separately for each of the sample duplicates. If the two results were discordant, the sample result was not included.

For the copy number assay, the same Net R Dye FOZ and Net F dye FOZ (CYP2D6/a-actin) were calculated as above and the ratio of the a-actin to CYP2D6 NET FOZ was calculated to identify CYP2D6 copy number (R Dye NET FOZ/F Dye NET FOZ, as with the allelic ratio formula above). To identify gene copy number the following cutoffs were used: a ratio less than 0.35 was scored as a single CYP2D6 gene copy, a ratio equal to or greater than 0.40 but less than 0.60 as two gene copies and a ratio equal to or greater than 0.65 as three gene copies. Ratios that fell within the two ranges (equal to or greater than 0.35 but less than 0.40 and equal to or greater than 0.60 but less than 0.65 were scored as equivocal and the sample result was not included.

Of the 181 genomic DNA samples used for the CYP2D6 gene amplification assays, 171 were detected by standard agarose gel electrophoresis. Out of the 10 DNAs that did not generate a visible PCR product three could still be detected by INVADER assays and were included in the analysis. The remaining seven DNAs were considered degraded and not used. All INVADER reactions were performed in duplicate and each reaction was scored independently for genotype. A final genotyping score was recorded only if the results for the duplicates were concordant. From a possible 1,914 results for the 11 loci and 174 DNA samples, 1,904 unambiguous genotyping scores were recorded; only ten genotyping scores could not be assigned. Of these ten assays, four were invalid because of low signal in both duplicates and six were invalid because signal ratios from both duplicates fell within the equivocal ranges. All ten invalid assays were from the three DNA samples that did not generate visible triplex PCR products. These results are 99.5% concordant overall, and 100% concordant if only those samples that produced a PCR product detectable by ethidium bromide staining are included.

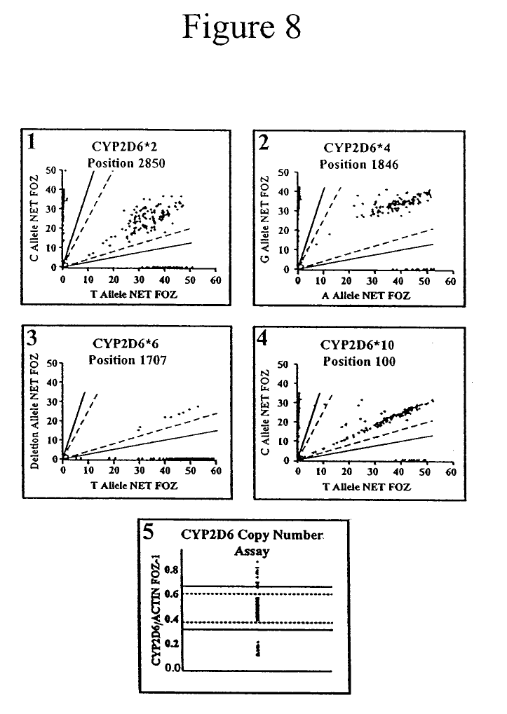

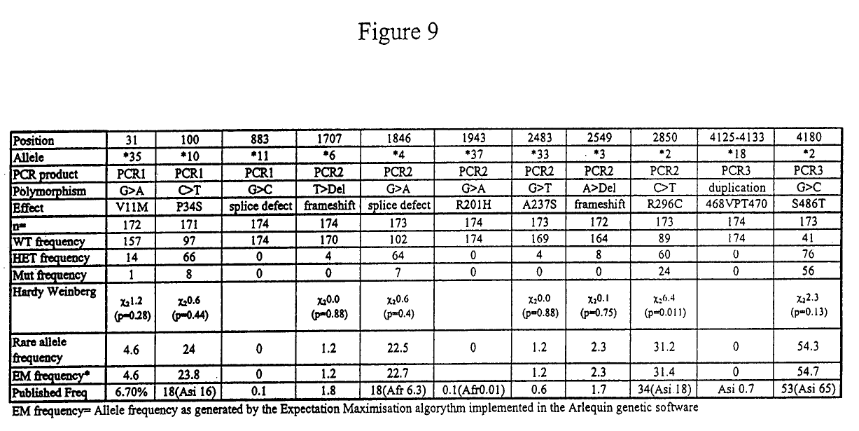

FIG. 8 contains four graphs as representative examples of the Net FOZ values (FOZ−1) from the 11 different assays; the resulting allele frequencies are presented in FIG. 9. All samples yielded heterozygous or homozygous variant signals except for CYP2D6*11-883, *18-4125 and *37-1943. These three alleles have a previously reported frequency of 0.1% or less (Marez, D., et al., Pharmacogenetics, 1997. 7(3):193); their absence in the 174 individuals analysed here is therefore not unexpected. The allele frequencies found in this study are comparable to published allele frequency values for Caucasians. The CYP2D6*2-2850 assay does not satisfy the Hardy-Weinberg equilibrium, however, Graph 1 in FIG. 8 shows a very clean separation of data. Further, all duplicates are in strong concordance and no unpredicted haplotypes were detected. With a relatively small sample size, a p value of 0.01 may be within acceptable limits.

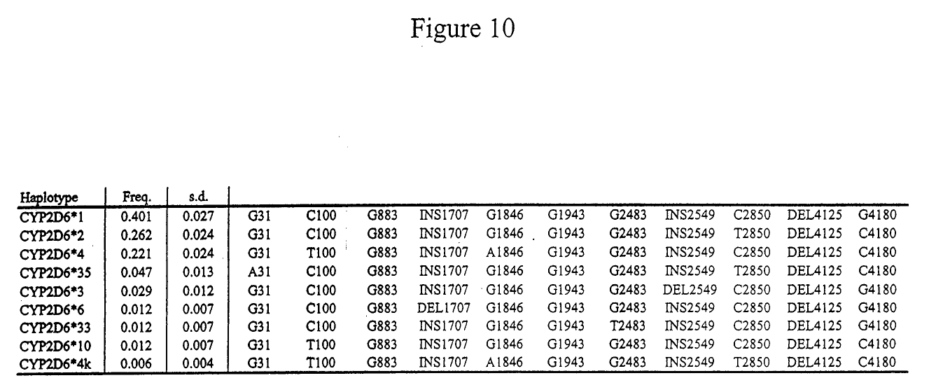

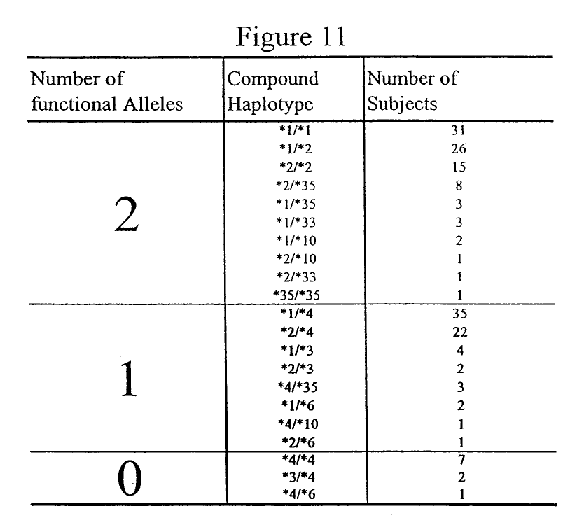

Individual CYP2D6 haplotypes were constructed using the Clarke method (Clark A G., 1990, Mol Biol Evol 7:111-122) with the aid of information on the web site (imm.ki.se/CYPalleles/cyp2d6.html) and compound haplotypes were assigned to individuals. Allele and haplotype frequencies were also independently calculated using the expectation maximisation (EM) algorithm implemented in the Arlequin software (http://lgb.unige.ch/arlequin/) (Schneider, S., D. Roessli, and L. Excoffier, Arlequin ver. 2.000: A software for population genetics data analysis. 2000, genetics and Biometry Laboratories, University of Geneva; Slatkin, M. and L. Excoffier, Heredity, 1996. 76(Pt 4):377). The EM algorithm identified 9 different haplotypes within the 172 samples that yielded concordant genotyping information (FIG. 10). These haplotypes co-segregated into 22 different compound haplotypes, as inferred by the Clarke method (FIG. 11). Ten individuals carried two non-functional CYP2D6 alleles and 70 individuals carried a single functional allele (FIG. 11).

The copy number assay (205 samples) identified 17 single-copy individuals, 170 two-copy individuals and 17 three-copy individuals. The results from one assay fell into the equivocal range described above. As Graph 5 in FIG. 8 clearly illustrates, the groupings of one, two, and three copies of the CYP2D6 gene are distinctly separated. Sixteen of the 17 gene deletions detected by the INVADER assay were confirmed by long-PCR. Eleven of the 17 gene duplications detected by the INVADER assay were confirmed by long-PCR. Generating lengthy PCR products requires pure and intact genomic DNA. Any fragmentation of the DNA template will lead to failure of PCR. Therefore, the absence of a long PCR product cannot in and of itself confirm the absence of the CYP2D6 duplication or deletion.

The INVADER assays provided an unambiguous genotyping determination for 100% of the 171 samples that yielded a visible PCR product on an agarose gel. The overall unambiguous genotyping determination was 95.9%, but this lower success rate can largely be attributed to PCR amplification failure. This poor PCR amplification most likely arises from partial degradation of the genomic samples used, because more than 40% of the same 181 samples also failed to yield a 5-kb PCR product in initial CYP2D6 long-range PCR amplification attempts. The triplex PCR approach described here will generate a CYP2D6-specific template from all but the most degraded DNA samples and may be more robust than protocols involving long-range PCR.

High failure rates inherent to long-PCR-based methods make the feasibility of using PCR-based methods to detect CYP2D6 copy number questionable. The accurate and automatable quantitative screening strategy we used to resolve CYP2D6 copy number alleles complements the PCR-INVADER genotyping assays well and avoids the problems associated with previous long-range PCR or RFLP methods.

In practice, this format is well suited to large-scale clinical trial or drug safety studies. It provides a rapid, comprehensive, high-throughput and ‘hands off’ method of achieving the high-resolution genotyping data needed to accurately predict CYP2D6 phenotypes. In addition, this preliminary study demonstrates the benefits of a clinical CYP2D6 genetic assay. Ten of the 174 DNA samples tested in the genotyping study possessed two non-functional alleles (FIG. 11) and 17 samples in the copy number study possessed a deleted allele. This information could be critical in a health care setting to avoid prescribing medications that are toxic at high doses. Equally, an individual homozygous for CYP2D6*35 may need higher doses of medication to achieve therapeutic levels due to the elevated enzyme activity observed in some *35 individuals. When prescribing medications to extensive metabolizers, health care providers should also consider whether potentially toxic metabolites would accumulate or whether a therapeutic level of medication would be reached. The quantitative nature of the INVADER assay is even more significant for extensive and ultra-rapid metabolizers. Complementing the PCR-INVADER genotyping assay with the genomic DNA copy number assay would be invaluable in identifying extensive and ultra-rapid metabolizers as well as the deleted alleles.

CASSETTE TABLE

| | | |
|---|---|---|
| *10 probe 2, *6 probe 1, | FRET 6, | Y-tct-X-tcg-gcc-ttt-tgg- |
| *4 probe 1, *3 probe 1, | Red/Z28, | ccg-aga-gac-ctc-ggc- |
| *2-2850 probe 2, *2-4180 | (931-74-10) | gcg-hex |
| probe 1, *18 probe 1, *11 | | (SEQ ID NO: 528) |
| probe 1, *35 probe 2, *33 | | |
| probe 2, *37 probe 2 | | |
| *10 probe 1, *6 probe 2, | FRET 7, | Y-tct-X-agc-cgg-ttt-tcc- |
| *4 probe 2, *3 probe 2, | FAM/Z28, | ggc-tga-gag-tct-gcc- |
| *2-2850 probe 1, *2-4180 | (931-74-02) | acg-tca-t-hex |
| probe 2, *18 probe 2, *11 | | (SEQ ID NO: 262) |
| probe2, *35 probe 1, *33 | | |
| probe 1, *37 probe 1 | | |

CYP 2D6 copy number

| | | |
|---|---|---|
| ACTIN PRIMARY | FRET 16, | Y-tct-X-agc-cgg-ttt-tcc- |
| PROBE | FAM/Z28, | ggc-tga-gac-ctc-ggc- |
| | (931-74-09) | gcg-hex |
| | & (1055-48-08) | (SEQ ID NO: 242) |
| 2D6 PRIMARY PROBE | FRET 13, | Y-tct-X-tcg-gcc-ttt-tgg- |
| | Red/Z28, | ccg-aga-gac-tcc-gcg- |
| | (1109-20-01) | tcc-gt-hex |
| | | (SEQ ID NO: 529) |

Y is FAM or RED
X is Z28
hex is hexane

Alternative Designs for the CYP2D6 INVADER Copy Number Assay

Additional experiments to determine the copy number of the cyp2D6*5 allele were carried out as described above with the following modifications. The CLEAVASE XI (Third Wave Technologies) enzyme (100 ng) was used in lieu of the CLEAVASE VIII enzyme. The following probe and INVADER oligonucleotide sequences were used in lieu of those listed in FIG. 7 (listed in FIG. 12).

CYP2D6*5
INVADER oligonucleotide:

(SEQ ID NO: 260)

5′-CCCGCGCCACCCACACTGAGCC
Probe oligonucleotide:

(SEQ ID NO: 261)

5′-ACGGACGCGGAGTTACAGCACAGGTGC.
Alpha actin internal control
INVADER oligonucleotide:

(SEQ ID NO: 263

5′-AGGAGTAGCCACGCTCGGTGAGGATCTTCATT
Probe oligonucleotide:
5′-CGCGCCGAGGCAGGTAGTCGGTGAGATC

### Example 4

### Comprehensive System for the Determination of Cytochrome p450 2D6 Genotypes

The following example provides a comprehensive system for the determination of cytochrome p450 2D6 genotypes. In some embodiments, the system provides a multi-step process to identify genotype and copy number of CYP2D6 polymorphisms. In some embodiments, the system uses a four step process (which can be conducted in any order that permits the results to be obtained), including the steps of: 1) determining the CYP2D6 gene copy number; 2) determining the genotype of specific SNPs in the CYP2D6 gene region; 3) perform reflex assays, if desired, to determine the copy number of some specific mutant alleles (as opposed to the whole gene); and 4) compare the experimental data obtained in steps 1-3 (or steps 1-2) to a matrix comprising: SNP genotype, copy number, and copy number of some specific mutant alleles vs. star allele designation. Use of such a system provides accurate and useful genotype information of the vast majority of known CYP2D6 polymorphisms. Preferred embodiments of the system are illustrated below employing the INVADER assay. It will be appreciated by skilled artisans that other detection assay technologies may also be employed.

In some embodiments of the present invention, the INVADER footprint region of the target sequence contains polymorphisms in addition to the single nucleotide polymorphism (SNP) of interest. In some embodiments, the additional target polymorphsims have no effect on phenotype. In some embodiments of the INVADER assay, sets of INVADER oligonucleotides with different sequences are used to detect a particular SNP of interest. In some embodiments, the sequence of a set of INVADER oligonucleotides differs from one another to account for the other SNP or SNPs that are not of interest, but that are near the SNP of interest. In other embodiments, each set of INVADER oligonucleotide differs from the other set of INVADER oligonucleotides by two or more bases. In some embodiments, two sets of INVADER oligonucleotides are used. In other embodiments, more than two sets of INVADER oligonucleotides are used.

1. Characterization of CYP2D6 Gene Copy Number Using the INVADER Assay System:

a. INVADER Assay Gene Copy Number Determination

The same INVADER assay methods for determining copy number described in the previous Examples were used (CYP2D6-specific oligonucleotides, and alpha-actin copy number control oligonucleotides). INVADER assays were performed in duplicate on 44 genomic samples prepared using the Gentra AUTOPURE LS® machine and Gentra PUREGENE™ chemistry, according to the manufacturer's instructions. Briefly, 7.5 μl of a genomic DNA (20-30 ng/μl) or a negative control (10 ng/μl tRNA) were added to the appropriate wells followed by addition of 7.5 μl of the appropriate primary probes/INVADER oligonucleotide/MgCl₂ mix. After the additions, each reaction was overlaid with 15 μl of molecular biology-grade mineral oil to prevent evaporation (Sigma-Aldrich, Steinheim, Germany). Each 15-μl reaction contained 80 ng CLEAVASE XI enzyme (Third Wave Technologies, Madison, Wis.), 2.5% PEG 8000, 2.5% glycerol, 0.025% NP 40, 0.025% TWEEN 20, 5.1 ug/ml BSA, 0.33 μM each of FAM-Arm1 dye (F dye) and RED-arm3 dye (R dye) FRET cassettes (FRET-16 and FRET-26; SEQ ID NOs: 242 and 243, respectively), 15.4 mM MgCl2, 0.5 μM of each allele-specific primary probe, and 0.05 μM INVADER oligonucleotide. Following reagent dispensing, plates were spun for 30 seconds at 1,000 rpm, then 63° C. for 4 hours using a MJ Thermocycler. Fluorescence was measured directly at the end of the incubation period using a CytoFluor 4000 fluorescence plate reader (Applied Biosystems, Foster City, Calif.). The settings used were 485/20 nm excitation/bandwidth and 530/25 nm emission/bandwidth for F dye detection and 560/20 nm excitation/bandwidth and 620/40 nm emission/bandwidth for R dye detection.

b. Copy Number Determination Calculations:

Data were exported into the Microsoft Excel program (Micorsoft, Redmond, Wash.). In this experiment, the CYP2D6 reports RED and the alpha actin control reports FAM. For each actin control signal and 2D6 signal, the Net Fold Over Zero (FOZ−1) values are calculated as follows:

$$\begin{matrix}Net F dye FOZ actin = \frac{F dye raw counts from sample}{\begin{matrix}F dye average raw counts \\ from negative controls\end{matrix}} - 1 \\ Net R dye FOZ 2 D6 = \frac{R dye raw counts from sample}{\begin{matrix}R dye average raw counts \\ from negative controls\end{matrix}} - 1\end{matrix}$$

Next, the ratio of the Net R dye FOZ value (2D6) to the Net F dye FOZ value (actin) for each sample DNA was calculated as follows:

$$Ratio R / F sample == \frac{Net R dye FOZ sample}{Net F dye FOZ sample}$$

Next, the Net Fold Over Zero (FOZ−1) value was calculated for the two copy, alpha actin/2D6 control genomic sample as follows (these values will be termed cNet FOZ F and cNet FOZ R):

$$\begin{matrix}c Net F dye FOZ alpha actin = \frac{F dye raw counts g control}{\begin{matrix}F dye average raw counts \\ from negative controls\end{matrix}} - 1 \\ c Net R dye FOZ alpha actin = \frac{R dye raw counts control}{\begin{matrix}R dye average raw counts \\ from negative controls\end{matrix}} - 1\end{matrix}$$

Next, the ratio of the cNet R dye FOZ value (2D6) to the cNet F dye FOZ value (actin) for the two copy alpha actin/2D6 genomic control was calculated as follows:

$$cRatio R / F alpha actin = \frac{cNet R dye FOZ 2 D6}{Net F dye FOZ alpha actin}$$

Finally, the sample Ratio R/F values were normalized as follows to yield “Ratio N” values, which correspond roughly to gene copy number:

$$Ratio N = \frac{Ratio \frac{R}{F} unknown sample}{Ratio \frac{R}{F} known 2 copy control} \times 2$$

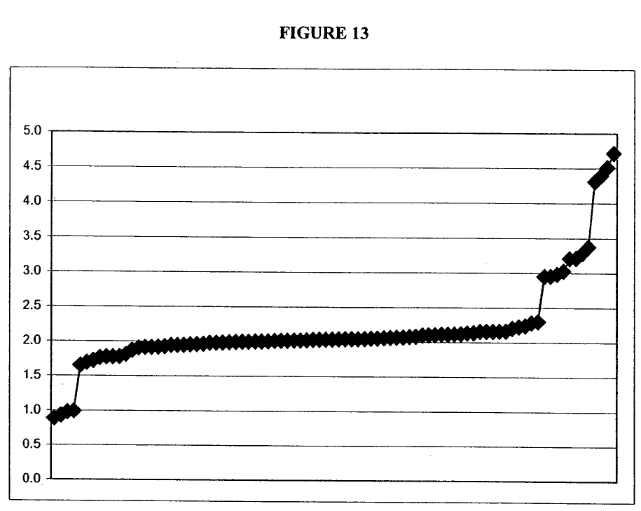

The Ratio N values can then be plotted. For example, FIG. 13 shows clusters of Ratio N values corresponding to copy number. In particular, FIG. 13 depicts samples with only one copy of the CYP2D6 gene range in Ratio N value from about 0.9 to about 1.1; samples with two copies range in Ratio N values from about 1.7 to about 2; samples with three copies range in Ratio N value from about 2.9 to about 3.4; and samples with four copies range in Ratio N value from about 4.3 to about 4.7.

2. Characterization of CYP2D6 Alleles Using a Tetraplex PCR and the INVADER Assay System

a. CYP2D6-Specific Tetraplex PCR for Genotyping

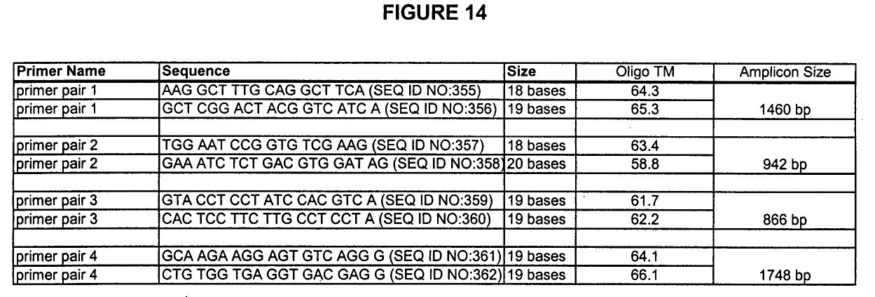

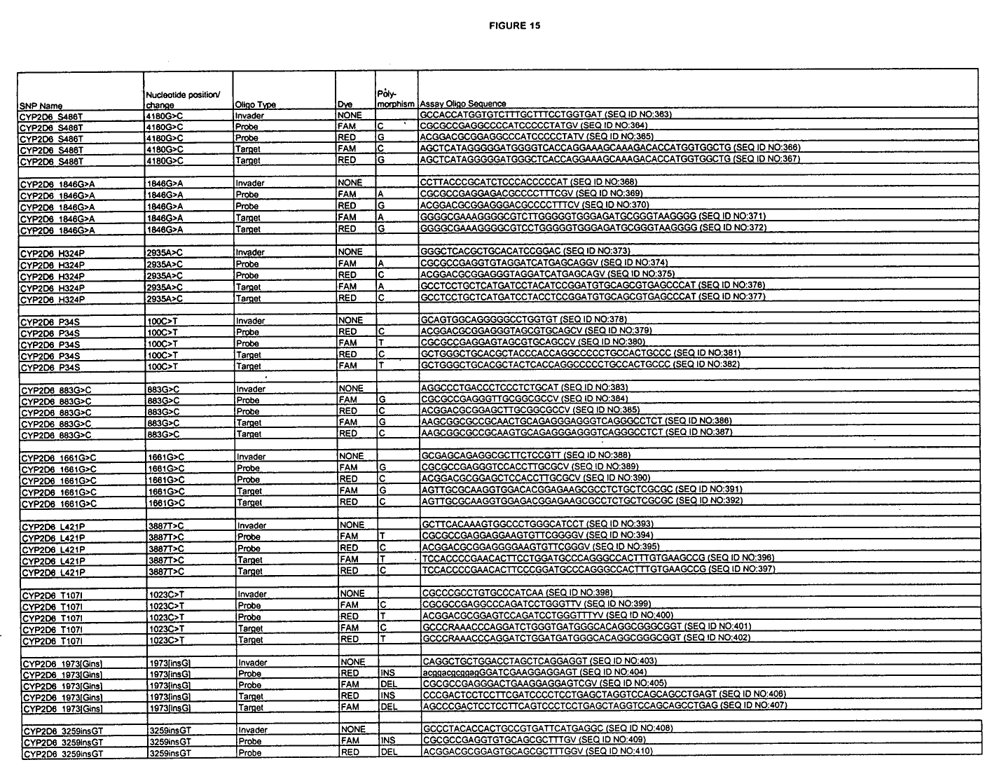

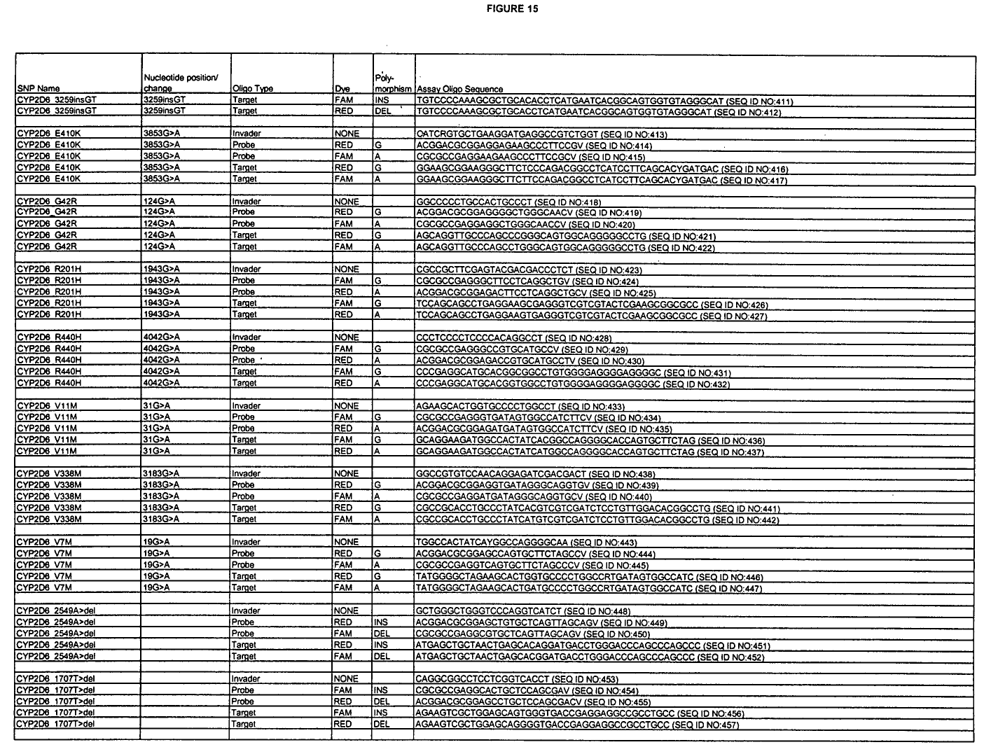

To further improve target quality and robustness of the PCR reaction, the CYP2D6 genomic region was divided into four shorter PCR fragments to pool together in a single tetraplex PCR reaction. All primers for the tetraplex-PCR reaction were designed over CYP2D6-specific sequence and are shown in FIG. 14. Primer pair 1 amplifies most of exons 1 and 2 and generates a product of about 1458 bp, primer pair 2 amplifies exons 3 and 4 and generates a product of about 950 bp, primer pair 3 amplifies exons 5 and 6 and generates a product of about 871 bp product, and primer pair 4 amplifies exons 7, 8 and 9 generating a 1752 bp product.

DNA from 44 leukocyte samples (from 44 anonymous donors) was isolated using the Gentra AUTOPURE LS machine and Gentra PUREGENE chemistry, according to the manufacturer's instructions. The CYP2D6-specific tetraplex reactions were performed using the ‘Herculase Hotstart’ PCR system (Stratagene, La Jolla, Calif. Cat. No. 600310), with 100-200 ng of genomic DNA, 250 μM dNTP's, 0.2 μM each primer, 2% DMSO, 1× Herculase Enzyme Buffer, and 2.5 units of enzyme in a final volume of 50 μL. The reaction was incubated on a ThermoHybaid PCR express Thermocycler (ThermoHybaid, Franklin, Mass.) using the following cycling parameters: 1) 95° C. for 10 minutes, 2) 94° C. for 30 seconds, 3) 55° C. for 1 minute, 4) 72° C. for 3 minutes, 5) 72° C. for 10 minutes, 6) 99° C. for 10 minutes, 7) hold at 4° C. Steps 2-4 were repeated for a total of 35 cycles. For verification purposes, 3 μL of the PCR product was visualized on a 1% agarose gel containing ethidium bromide. The PCR products were diluted 1/50 in 10 μg/ml brewer yeast tRNA in TE, pH 7.5.

b. INVADER CYP2D6 Genotyping Assays

INVADER assays were designed for the following CYP2D6 polymorphisms: 19G>A, 31G>A, 100C>T, 124G>A, 221C>A, 833G>C, 984A>G, 1023C>T, 1039C>T, 1661G>C, 1707T>del, 1758G>A, 1758G>T, 1846G>A, 1863ins[TTTCGCCCC]2, 1943G>A, 1973insG, 2539-2542delAACT, 2549A>del, 2573insC, 2613-2615delAGA, 2850C>T, 2935A>C, 3183G>A, 3259insGT, 3853G>A, 3887T>C, 4042G>A, 4180G>C. FIG. 6B represents the relative positions of most of these assays. Each PCR product was detected by at least two INVADER assays. FIG. 15 provides the sequences for the oligonucleotides. The F and R dye FRET cassettes used in these experiments were those described in Example 3 (SEQ ID NOs:242 and 243). Each assay used a synthetic target complementary to both the probe and INVADER oligonucleotides as a positive control. The INVADER assay reactions were performed in 96-well plates. Each INVADER assay was tested against all 44 different DNA donor samples.

In some embodiments, it is possible to detect polymorphisms that are in linkage with the desired polymorphism to be detected. For example, if any particular polymorphism proves difficult to work with (e.g., because a detection assay technology is incapable of detecting particular classes of polymorphisms—e.g., deletions, insertions, repeats), substitution of linked polymorphisms may be used to identify the presence of the desired polymorphism. For example, 2573insC is linked to 221C>A and 223C>G. Either or both of221C>A and 223C>G may be used as a substitute for detecting 2573insC. In other embodiments, 2573insC may be detected directly, e.g. using oligos in FIG. 12 (SEQ ID NOs: 265-269).

Briefly, 7.5 μl of a 1/50 to 1/100 dilution of the CYP2D6-specific PCR products or a negative control (10 ng/μl tRNA) were added to the appropriate wells followed by addition of 7.5 μl of the appropriate primary probes/INVADER oligonucleotide/MgCl₂ mix. After the additions, each reaction was overlaid with 15 μl of molecular biology-grade mineral oil to prevent evaporation (Sigma-Aldrich, Steinheim, Germany). Each 15-μl reaction contained 10 ng CLEAVASE XI enzyme, 2.5% PEG 8000, 2.5% glycerol, 0.025% NP 40, 0.025% TWEEN 20, 5.1 ug/ml BSA, 0.33 μM each of FAM-Arml dye and RED-arm3 dye FRET cassettes (FRET-16 and FRET-26 respectively), 15.4 mM MgCl2, 0.5 μM of each allele-specific primary probe, and 0.05 μM INVADER oligonucleotide. Following reagent dispensing, plates were spun for 30 seconds at 1,000 rpm, then incubated at 63° C. for 40 minutes using a thermocycler. Fluorescence was measured directly at the end of the incubation period using a CytoFluor 4000 fluorescence plate reader (Applied Biosystems, Foster City, Calif.). The settings used were 485/20 nm excitation/bandwidth and 530/25 nm emission/bandwidth for F dye detection and 560/20 nm excitation/bandwidth and 620/40 nm emission/bandwidth for R dye detection.

C. Data Analysis for the Tetraplex PCR—INVADER Genotyping Assay

Data were exported into the Microsoft Excel program (Microsoft, Redmond, Wash.). For each allele of a given polymorphism, the Net Fold Over Zero (FOZ−1) values are calculated as follows:

$$\begin{matrix}Net F dye FOZ = \frac{F dye raw counts from sample}{\begin{matrix}F dye raw counts \\ from negative control\end{matrix}} - 1 \\ Net R dye FOZ = \frac{R dye raw counts from sample}{\begin{matrix}R dye raw counts \\ from negative controls\end{matrix}} - 1\end{matrix}$$

Determination of the genotype or copy number was based on the ratio of the Net R dye FOZ value to the Net F dye FOZ value as shown below:

$$Allelic Ratio = \frac{Net R dye FOZ}{Net F dye FOZ}$$

In cases where the Net FOZ value was equal to or less than zero, the Net FOZ value was adjusted to 0.01 to avoid the generation of negative values or division by zero. An allelic ratio of equal to or less than 0.25 was scored as homozygous for the F dye allele, a ratio greater than 4 was scored as homozygous for the R dye allele and a ratio greater than 0.25 but less than 4 was scored as heterozygous. Values that fell within two ranges (greater than about 0.2-0.25 and equal to or less than about 0.3-0.4, or equal to or greater than about 2.5-3.3 and equal to or less than about 4-5) were designated as equivocal and the sample result was not included.

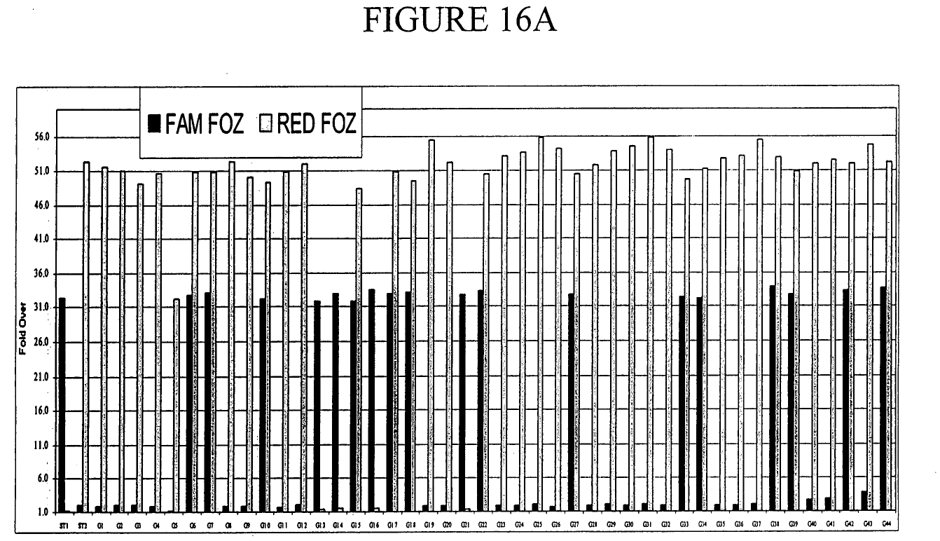

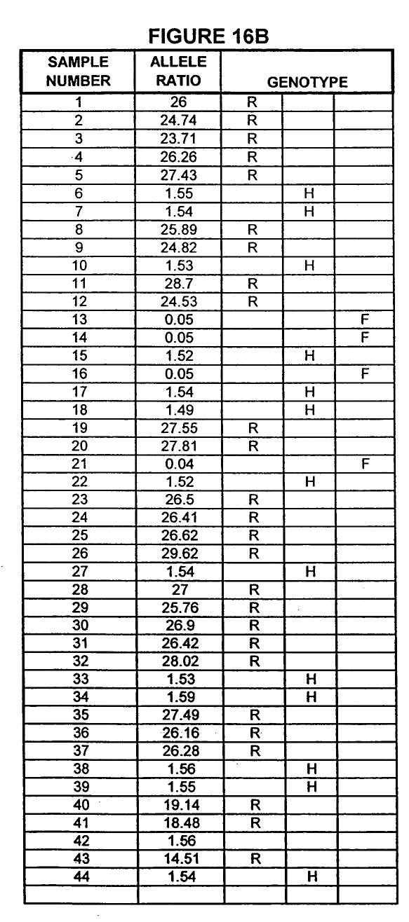

Instances in which both the F dye and R dye Net FOZ values were less than 2, were recorded as low-signal. The allelic ratio was calculated separately for each of the sample duplicates. If the two results were discordant, the sample result was not included. FIG. 16A shows the Net FOZ data of 44 samples tested with the 100C>T INVADER assay. The dark bar represents Net FAM dye FOZ, the light bar represents Net RED dye FOZ. FIG. 16B shows the allele ratios and the genotype calls. R=homozygous RED (wild-type at the 100C>T locus); H=heterozygous at the 100 C>T locus; F=homozygous FAM (mutant at the 100C>T locus).

3. Reflex Assays

For the subset of samples that contain more than two copies of the CYP2D6 gene, a reflex test can be performed to distinguish between different types of allele duplications. Of the 43 different CYP2D6 alleles characterized to date, only four alleles (*1, *2, *4, and *35) are present in individuals having a duplication of the entire CYP2D6 gene or large portions of the CYP2D6 gene. Three SNP's, 31G>A, 100C>T, and 4180G>C, are the minimum number suggested to distinguish between duplications of these four alleles. Of these four alleles, only *35 carries the 31G>A mutation; *1, 2 and *4 do not. Similarly, only the *4 allele carries the 100C>T mutation; neither *2 nor *35 do. Finally, all but the *1 and *4J allele carry the 4180G>C mutation; however, if neither the 31G>A nor the 100C>T mutations were detected in the SNP assay, then only the number of copies of the 4180G>C mutation need be determined for a final genotype call (e.g. determine if the sample has one copy of *1 and two copies of *2 or two copies of *2 and only one copy of *1). In general, only those samples which have greater than two copies of the CYP2D6 gene as determined by the copy number assay, and are also heterozygous for 31G>A, 100C>T, or 4180G>C will gain maximum benefit from reflex testing.

In the following example, 43 genomic samples that were tested with the copy number assay and the INVADER SNP 100C>T assay were also tested with the 100T reflex assay. Reaction conditions for the 100T reflex assay were as described in the copy number assay in Example 4(1)(a) above.

Reflex copy number (100T) was determined as follows. Data were exported into the Microsoft Excel program (Micorsoft, Redmond, Wash.). In this experiment, the 100T reports RED and the alpha actin reports FAM. For each sample, the Net Fold Over Zero (FOZ−1) values are calculated for the actin signal and 100T signal as follows:

$$Net F dye FOZ ( actin ) = \frac{F dye raw counts from sample}{\begin{matrix}F dye average raw counts from \\ negative controls\end{matrix}} - 1$$

$$Net R dye FOZ ( 100 T ) = \frac{R dye raw counts from sample}{\begin{matrix}R dye average raw counts from \\ negative controls\end{matrix}} - 1$$

Next, the ratio of the Net R dye FOZ value (100T) to the Net F dye FOZ value (actin) for each sample DNA was calculated as follows:

$$Ratio R \text{/} F sample = \frac{Net R dye FOZ sample}{Net F dye FOZ sample}$$

In this experiment, each sample was run in duplicate. The Ratio R/F for the duplicates was averaged. This is called “Mean(Ratio)” and is shown in Column 4 of Table 1, below.
Next, the Net Fold Over Zero (FOZ−1) value was calculated for a known 1 copy 100T genomic control as follows (these values will be termed cNet FOZ F and cNet FOZ R):

$$cNet F dye FOZ ( 1 copy ) = \frac{F dye raw counts ( 1 copy T )}{\begin{matrix}F dye average raw counts \\ from negative controls\end{matrix}} - 1$$

$$cNet R dye FOZ alpha actin = \frac{R dye raw counts ( actin )}{\begin{matrix}R dye average raw counts \\ from negative controls\end{matrix}} - 1$$

Next, the ratio of the cNet R dye FOZ value (1 copy T) to the cNet F dye FOZ value (actin) for the one copy T genomic control was calculated as follows:

$$cRatio R \text{/} F 1 copy = \frac{cNet R dye FOZ ( 1 copy )}{cNet F dye FOZ ( actin )}$$

Finally, the sample Ratio R/F values were normalized as follows to yield “CN100T” values:

$$CN100T = \frac{Ratio R \text{/} F unknown sample}{cRatio R \text{/} F 1 copy}$$

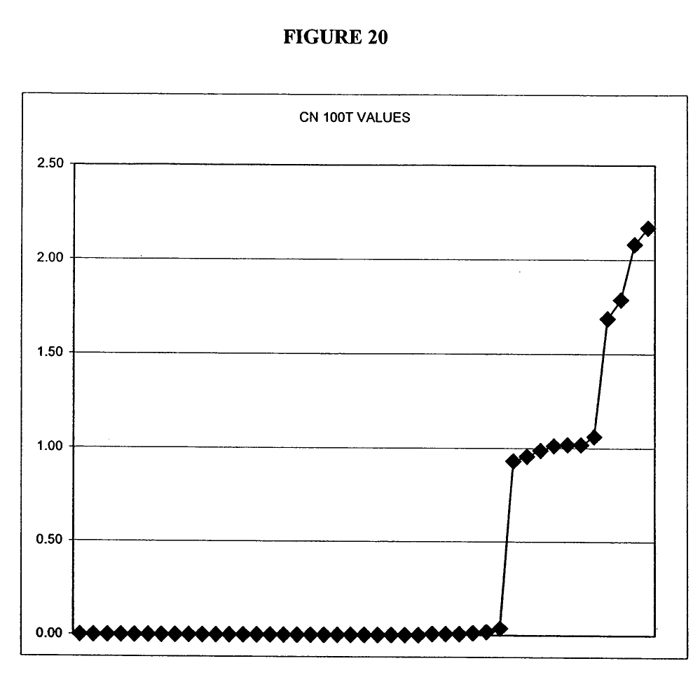

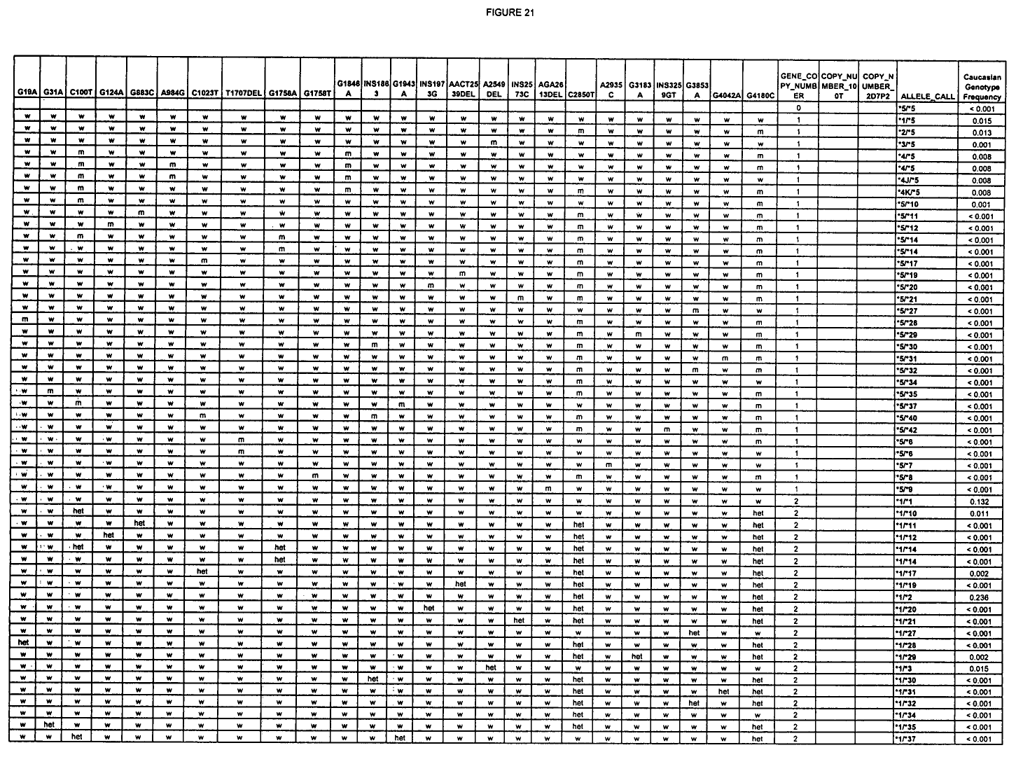

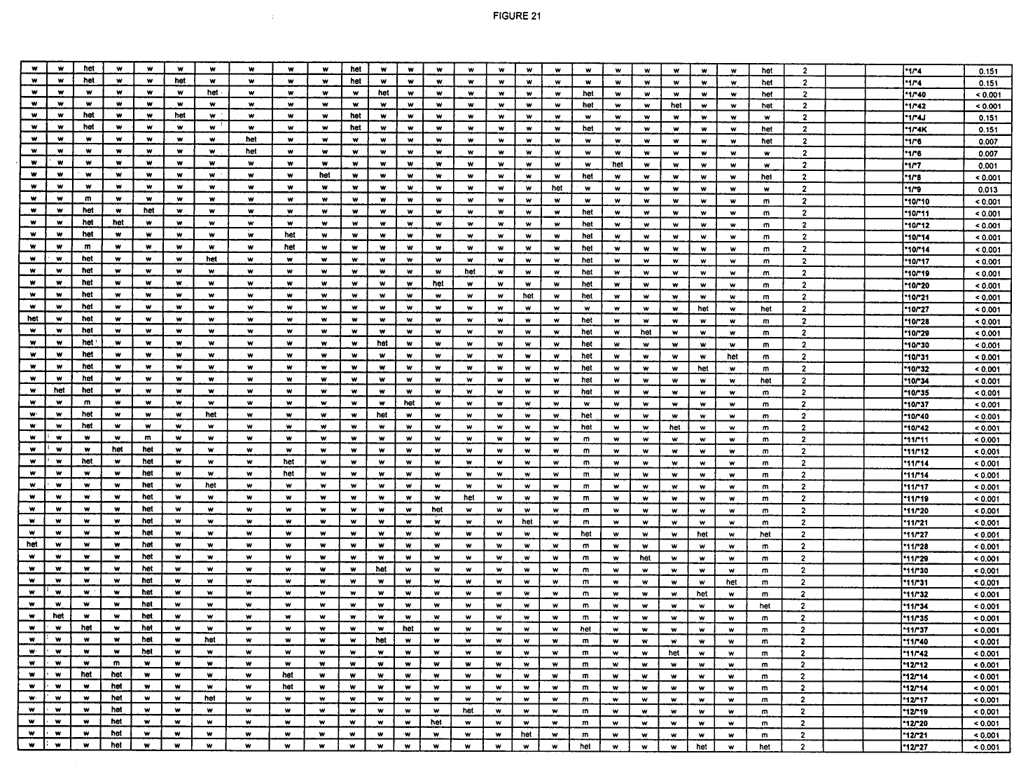

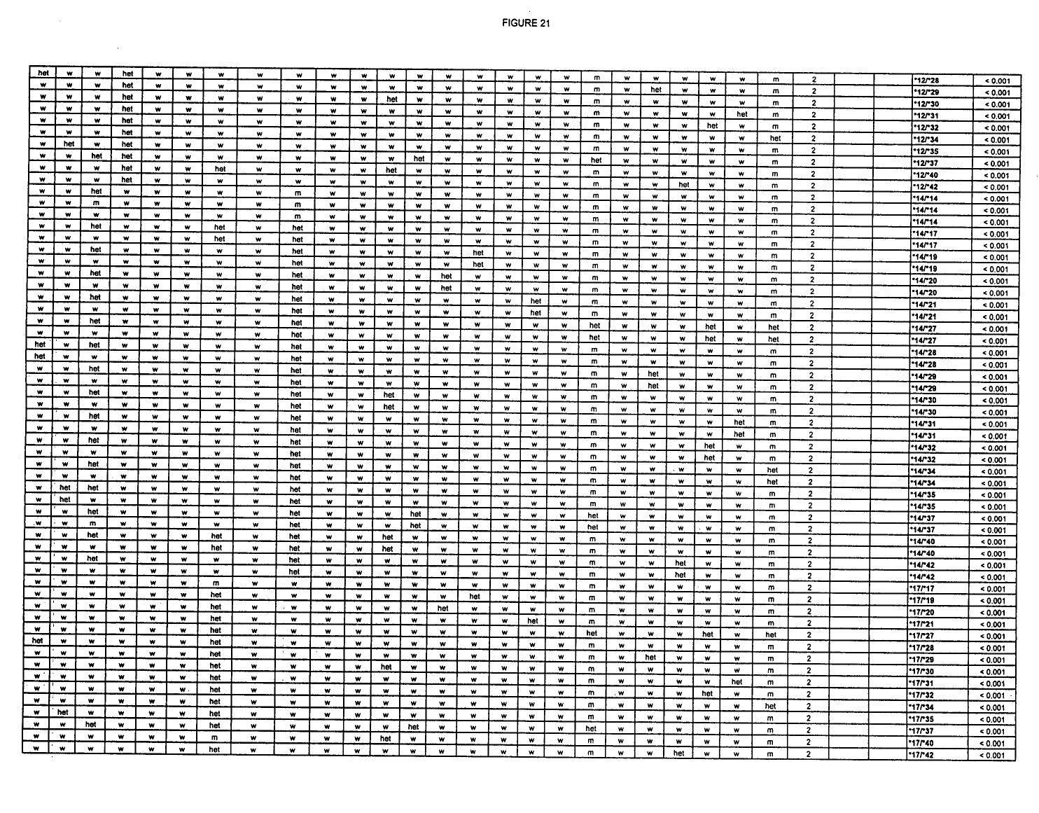

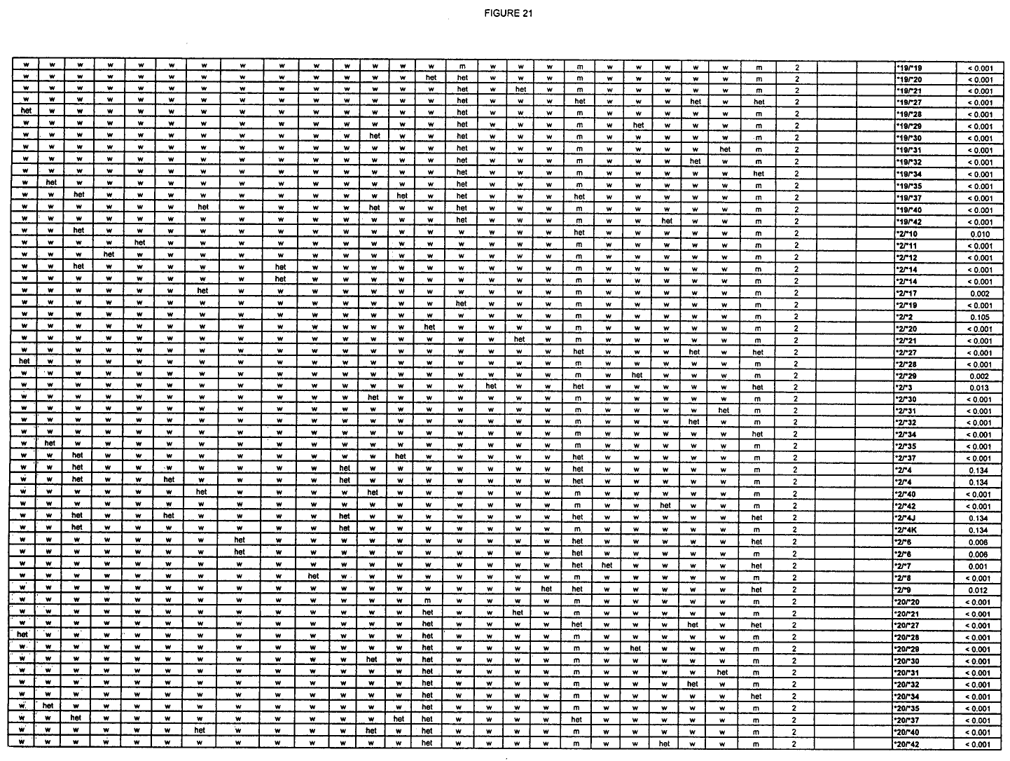

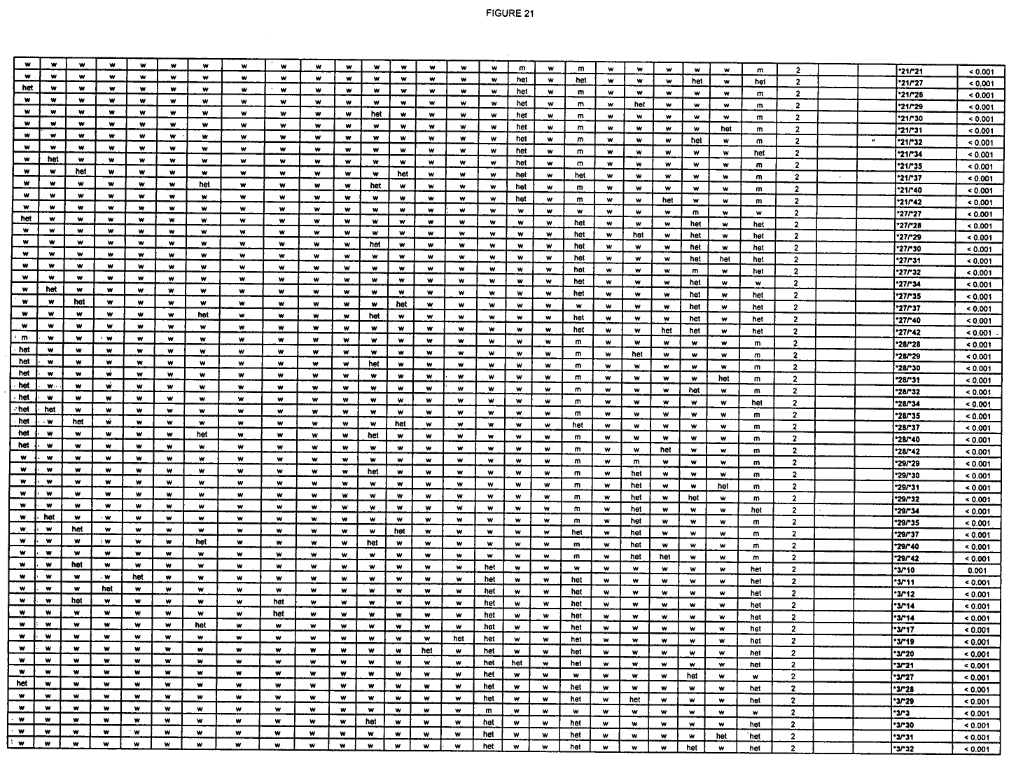

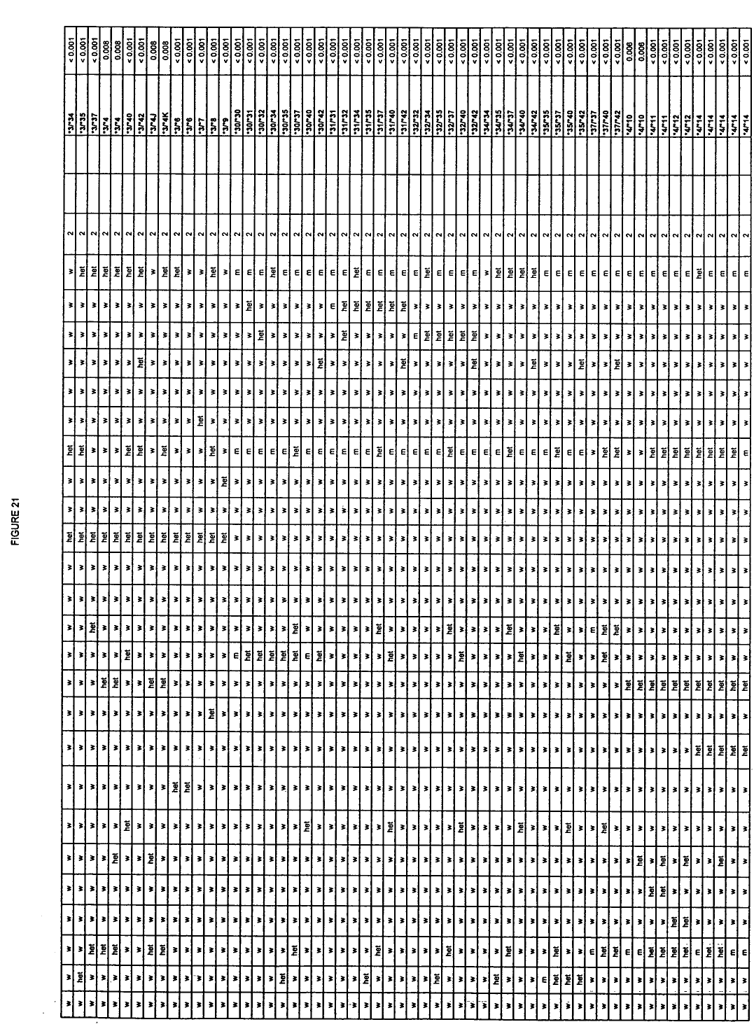

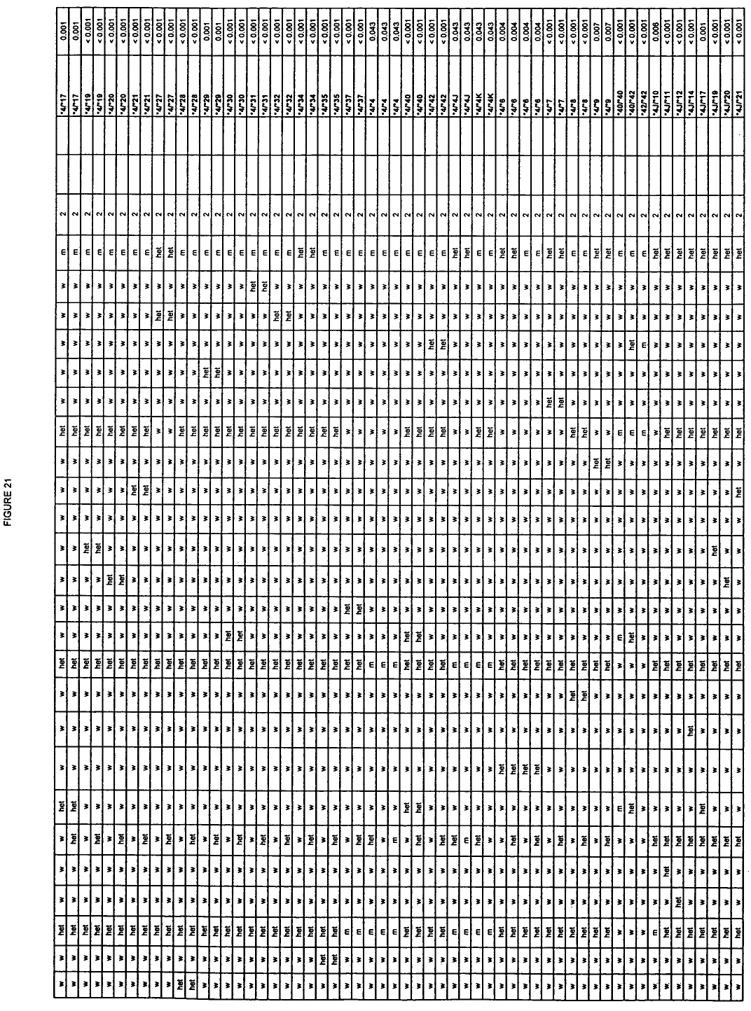

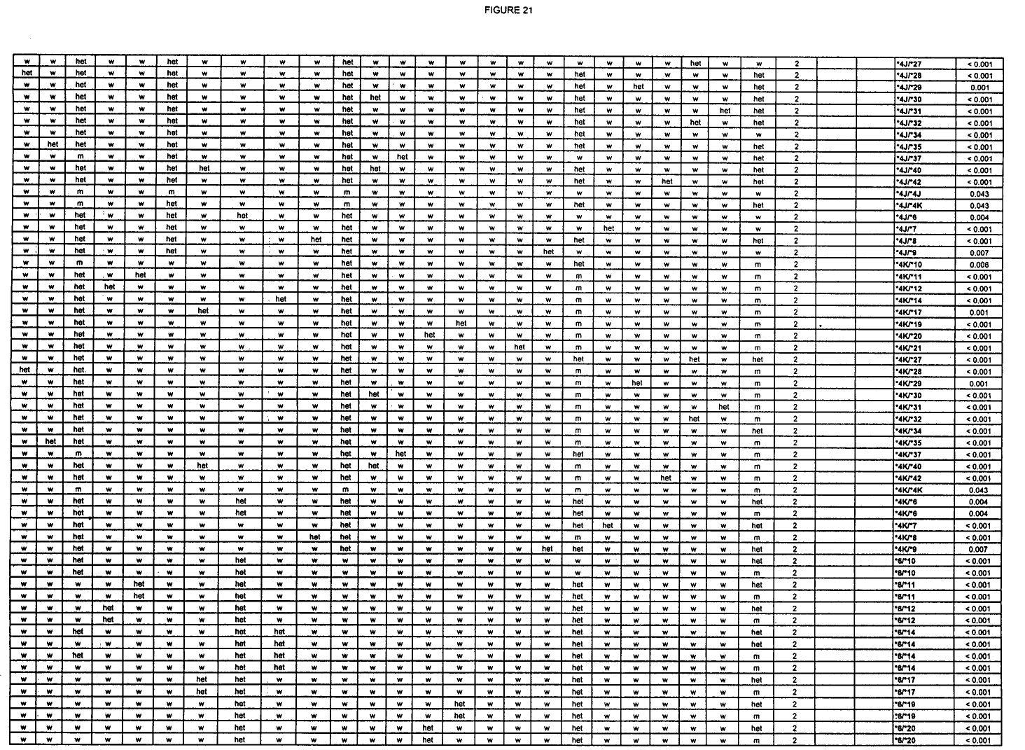

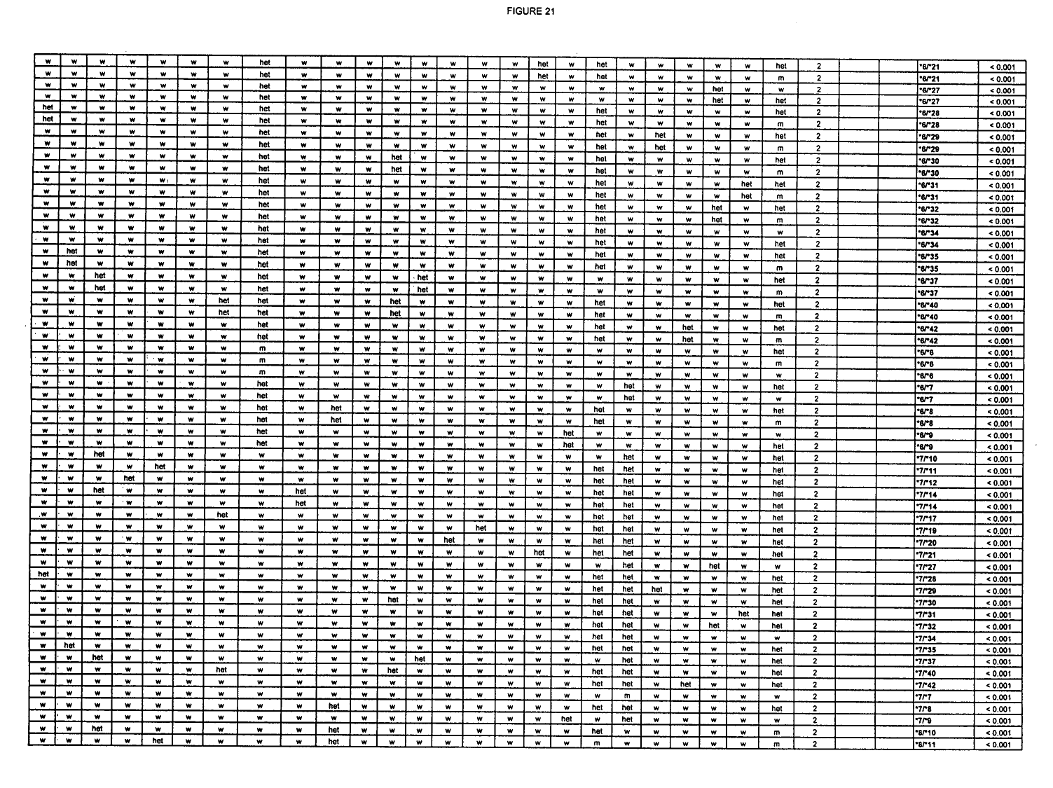

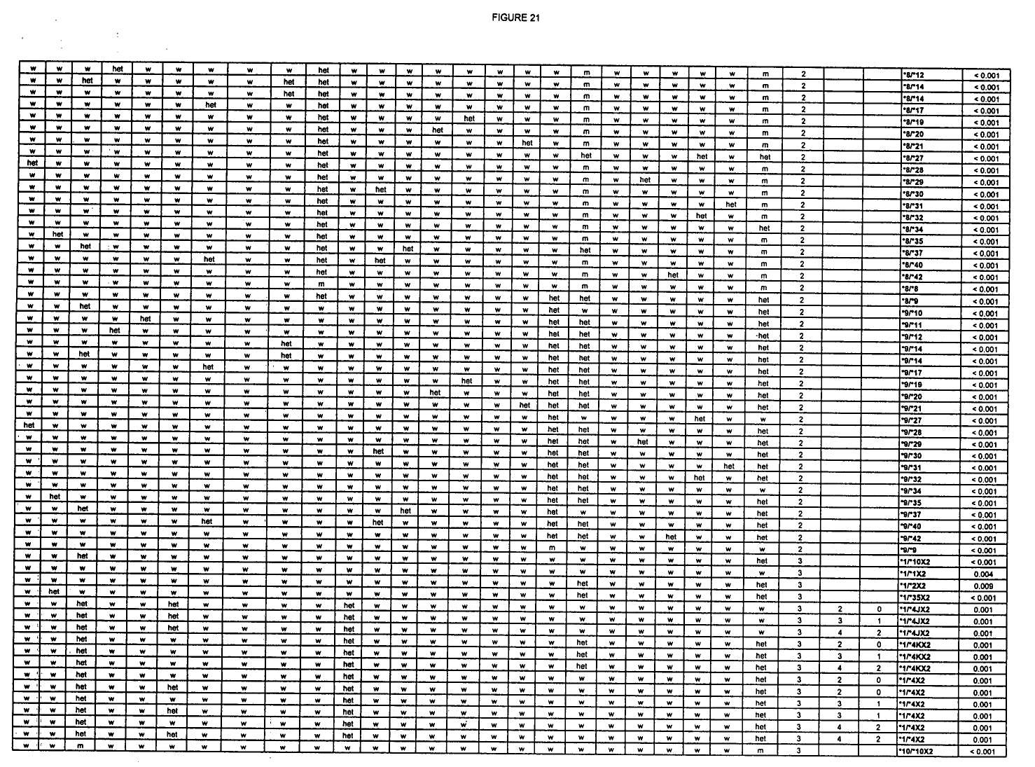

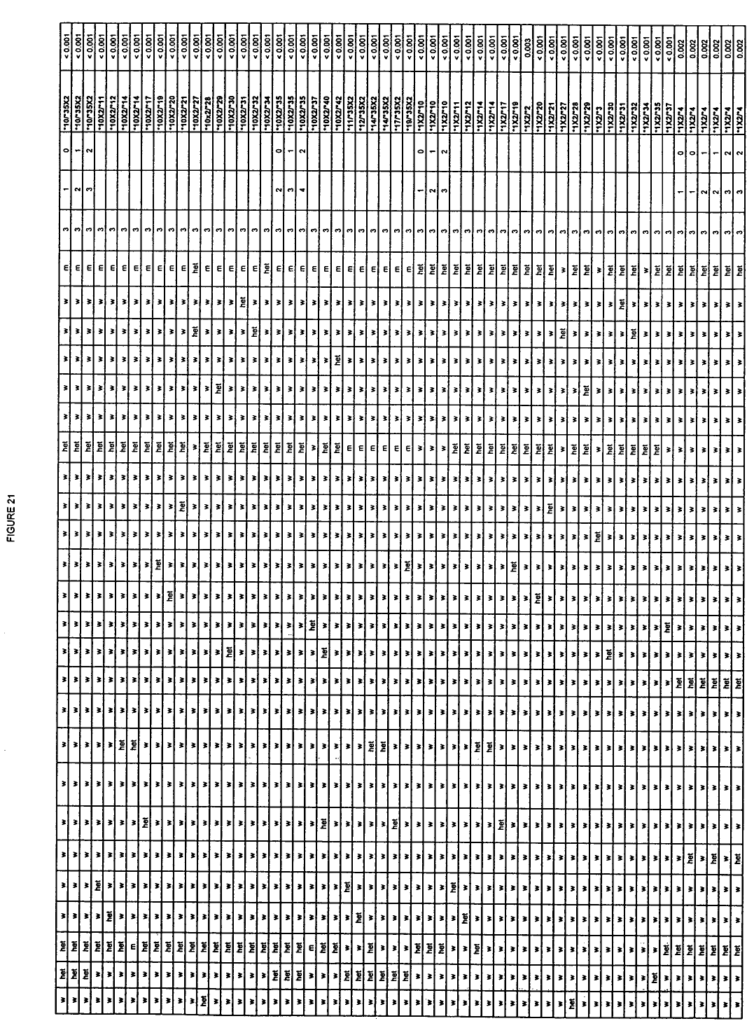

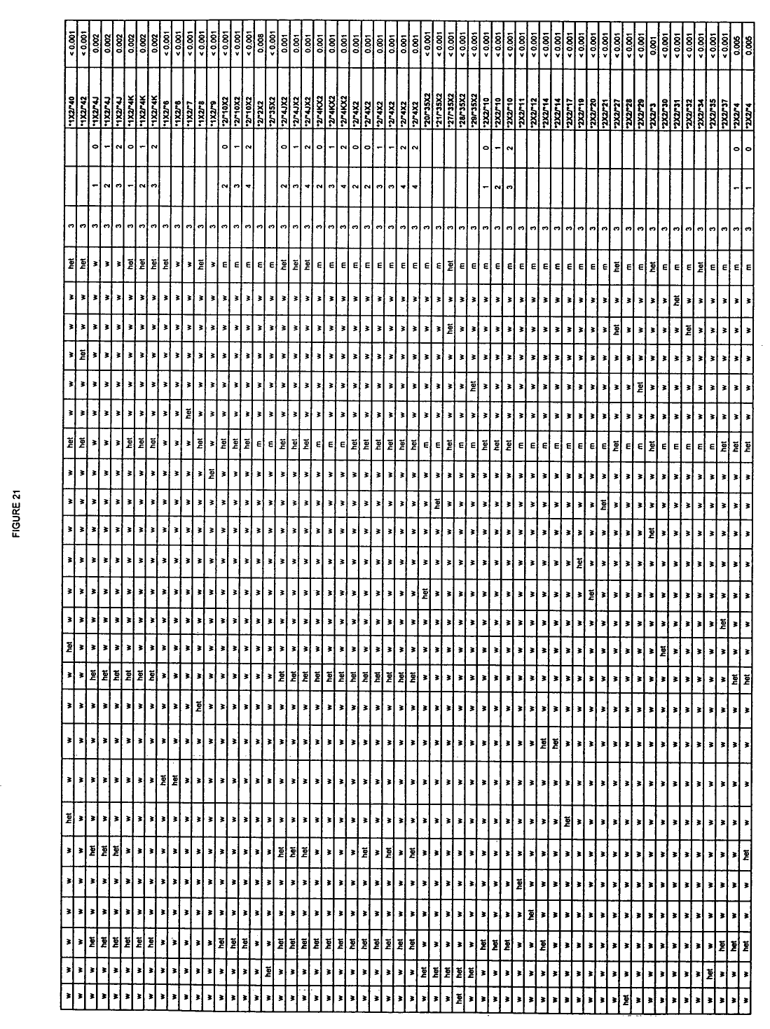

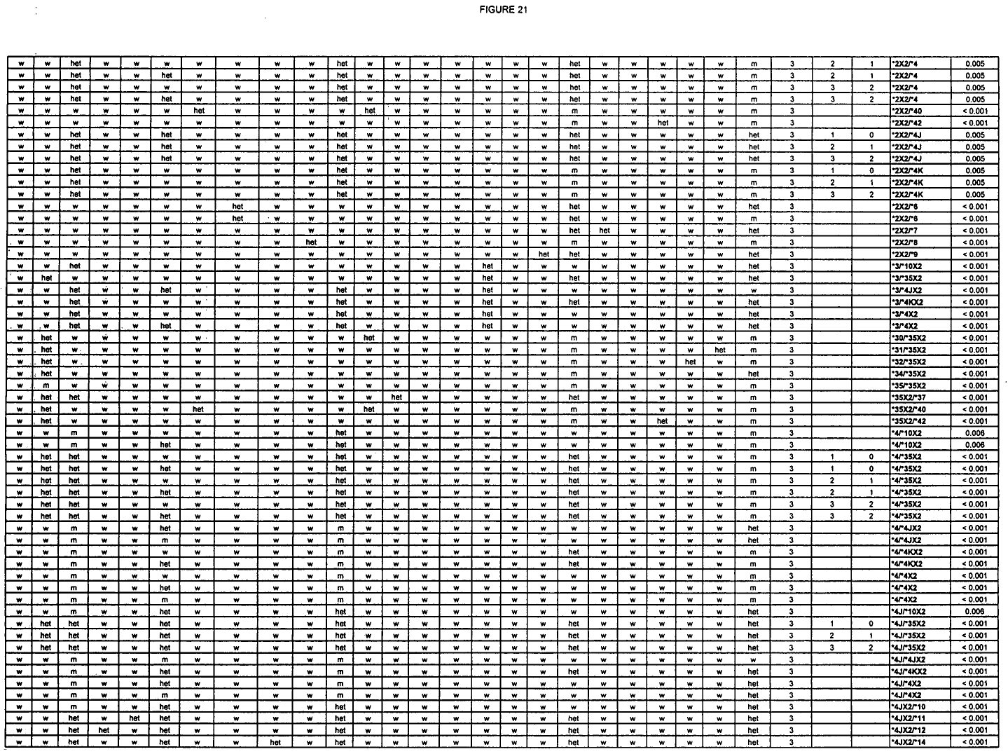

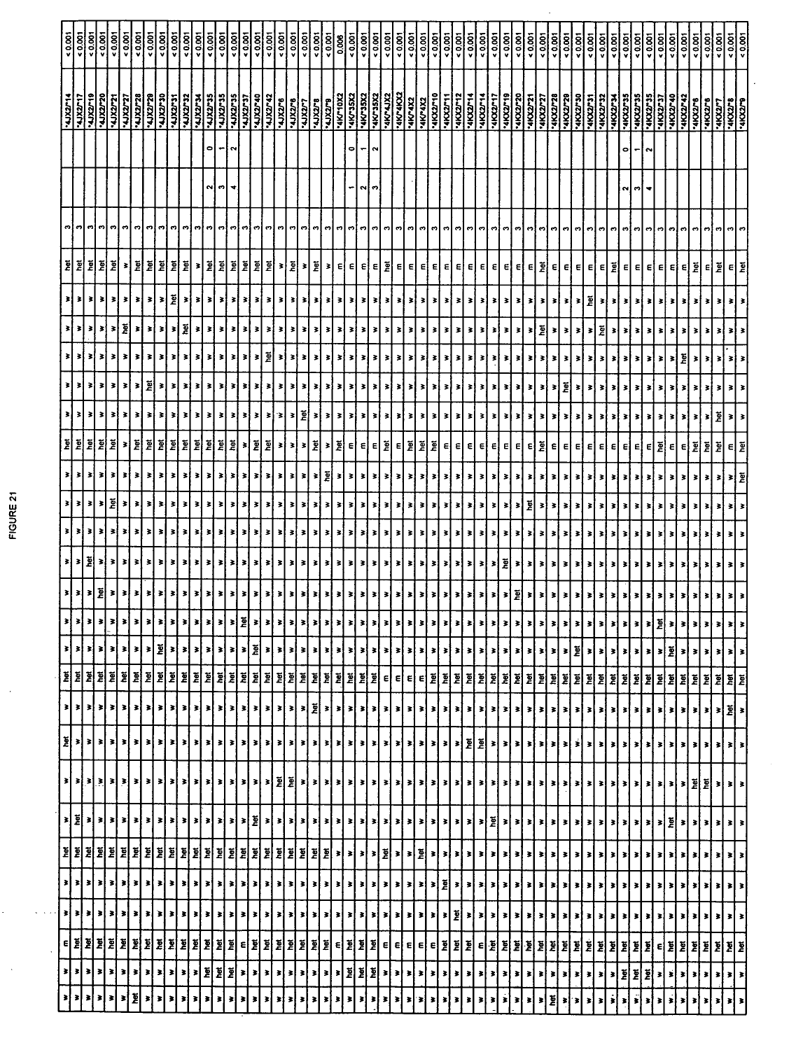

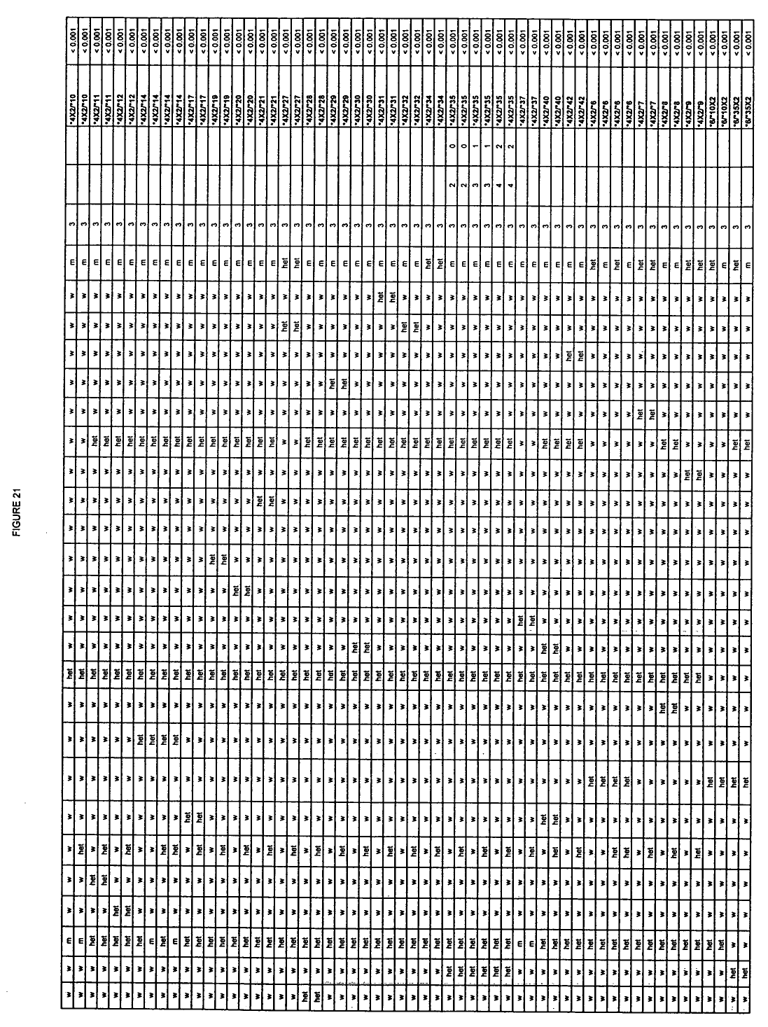

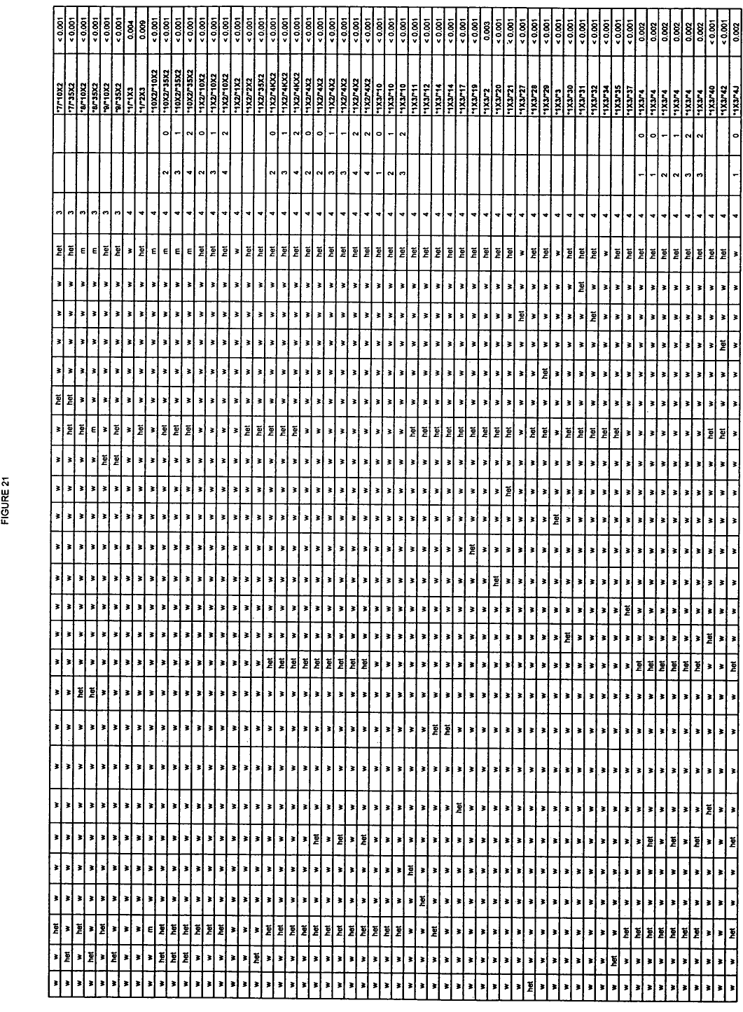

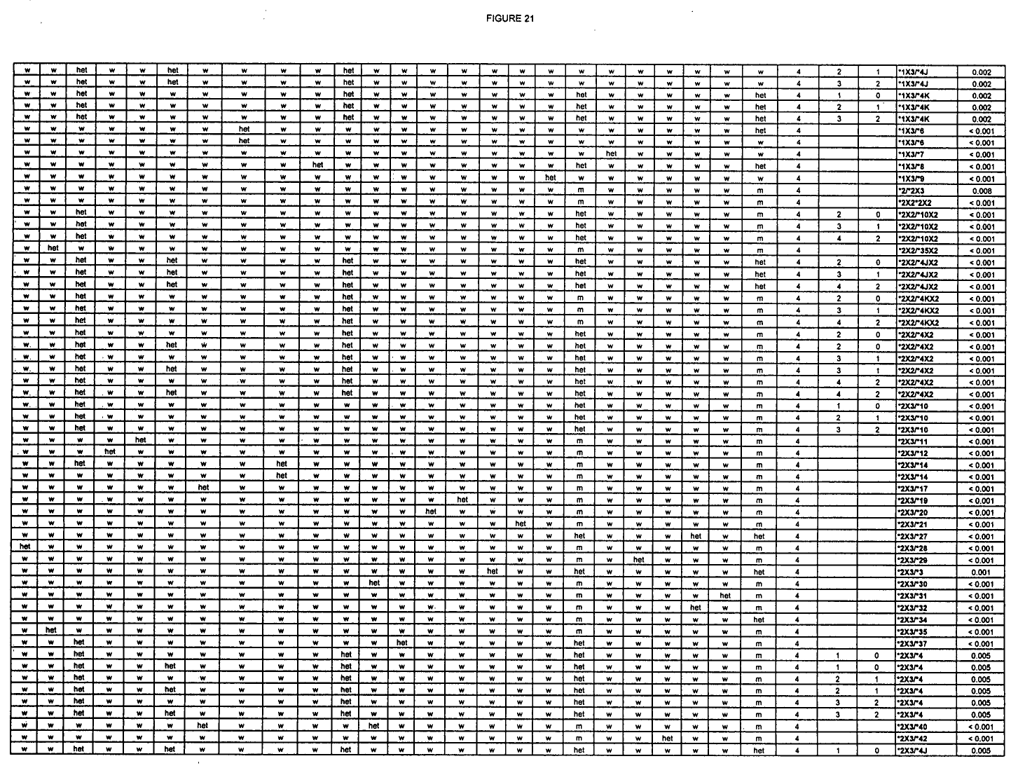

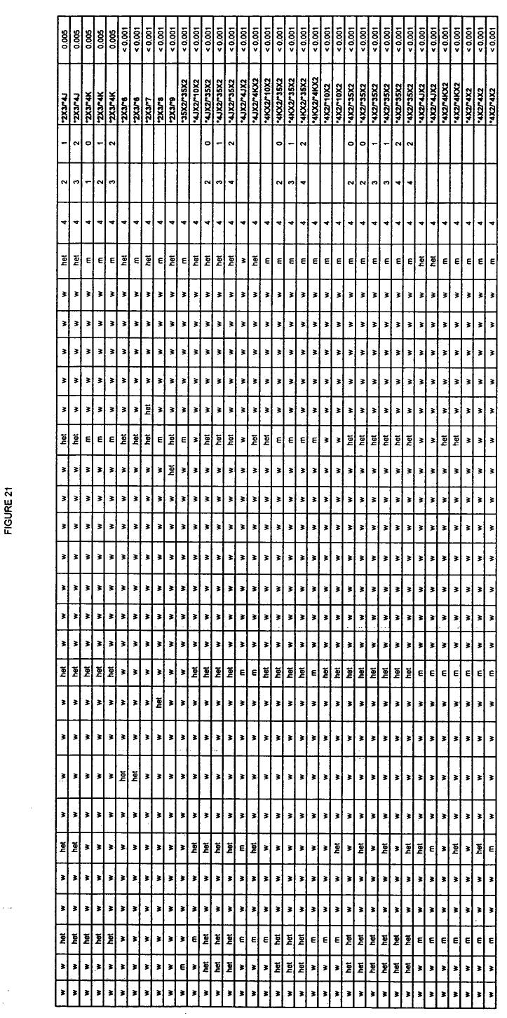

In Table 1, the sample number is shown in column 1, the 2D6 copy number call in column 2, the PCR-INVADER SNP 100C>T call in column 3, the Mean(Ratio) in column 4, the CN100T value in column 5, the copy number T call (as determined by evaluating the graph in FIG. 20) in column 6, and the genotype of the sample for this particular SNP position in column 7.

The CN100T values can be plotted (see FIG. 20) to better evaluate copy number. FIG. 20 shows clusters of CN100T values corresponding to copy number. In particular, FIG. 20 depicts samples with no copies of the 100T sequence range in CN100T value from about 0.0 to about 0.3; samples with one copy range in CN100T value from about 0.75 to about 0.85; samples with two copies range in CN100T value from about 1.35 to about 1.43; and samples with three copies range in CN100T value from about 2.08 to about 2.17.

TABLE 1

| Sample | 2D6 CN | 100CT Call | Mean(Ratio) | CN 100T Ratio | CN 100T | 100T Call |
|---|---|---|---|---|---|---|
| G45 | 2 | HMZ C | 0.00 | 0.00 | 0 | CC |
| G47 | 2 | HMZ C | 0.00 | 0.00 | 0 | CC |
| G49 | 2 | HMZ C | 0.00 | 0.00 | 0 | CC |
| G50 | 1 | HMZ C | 0.00 | 0.00 | 0 | C |
| G51 | 2 | HMZ C | 0.00 | 0.00 | 0 | CC |
| G52 | 2 | HMZ C | 0.00 | 0.00 | 0 | CC |
| G53 | 2 | HMZ C | 0.00 | 0.00 | 0 | CC |
| G54 | 2 | HMZ C | 0.00 | 0.00 | 0 | CC |
| G55 | 1 | HMZ C | 0.00 | 0.00 | 0 | C |
| G58 | 2 | HMZ C | 0.00 | 0.00 | 0 | CC |
| G60 | 2 | HMZ C | 0.00 | 0.00 | 0 | CC |
| G61 | 2 | HMZ C | 0.00 | 0.00 | 0 | CC |
| G62 | 2 | HMZ C | 0.00 | 0.00 | 0 | CC |
| G63 | 2 | HMZ C | 0.00 | 0.00 | 0 | CC |
| G65 | 2 | HMZ C | 0.00 | 0.00 | 0 | CC |
| G71 | 2 | HMZ C | 0.00 | 0.00 | 0 | CC |
| G73 | 2 | HMZ C | 0.00 | 0.00 | 0 | CC |
| G74 | 2 | HMZ C | 0.00 | 0.00 | 0 | CC |
| G76 | 1 | HMZ C | 0.00 | 0.00 | 0 | C |
| G78 | 2 | HMZ C | 0.00 | 0.00 | 0 | CC |
| G82 | 2 | HMZ C | 0.00 | 0.00 | 0 | CC |
| G83 | 2 | HMZ C | 0.00 | 0.00 | 0 | CC |
| G84 | 2 | HMZ C | 0.00 | 0.00 | 0 | CC |
| G85 | 2 | HMZ C | 0.00 | 0.00 | 0 | CC |
| G86 | 3 | HMZ C | 0.00 | 0.00 | 0 | CCC |
| G87 | 2 | HMZ C | 0.00 | 0.00 | 0 | CC |
| G48 | 2 | HMZ C | 0.01 | 0.01 | 0 | CC |
| G66 | 2 | HMZ C | 0.01 | 0.01 | 0 | CC |
| G79 | 2 | HMZ C | 0.01 | 0.01 | 0 | CC |
| G77 | 2 | HMZ C | 0.01 | 0.01 | 0 | CC |
| G46 | 2 | HMZ C | 0.02 | 0.02 | 0 | CC |
| G88 | 2 | HMZ C | 0.03 | 0.04 | 0 | CC |
| G81 | 2 | HET | 0.75 | 0.94 | 1 | CT |
| G64 | 2 | HET | 0.77 | 0.97 | 1 | CT |
| G67 | 2 | HET | 0.79 | 1.00 | 1 | CT |
| G68 | 3 | HET | 0.81 | 1.03 | 1 | CCT |
| G69 | 2 | HET | 0.82 | 1.03 | 1 | CT |
| G75 | 2 | HET | 0.82 | 1.03 | 1 | CT |
| G72 | 2 | HET | 0.85 | 1.08 | 1 | CT |
| G80 | 2 | HMZ T | 1.35 | 1.71 | 2 | TT |
| G57 | 3 | HET | 1.43 | 1.81 | 2 | CTT |
| G59 | 3 | HMZ T | 1.67 | 2.11 | 3 | TTT |
| G56 | 3 | HMZ T | 1.74 | 2.20 | 3 | TTT |

In alternative embodiments, reflex testing is carried out using different methods, such as DNA sequencing or allele-specific PCR.

4. Determination of CYP2D6 Genotype

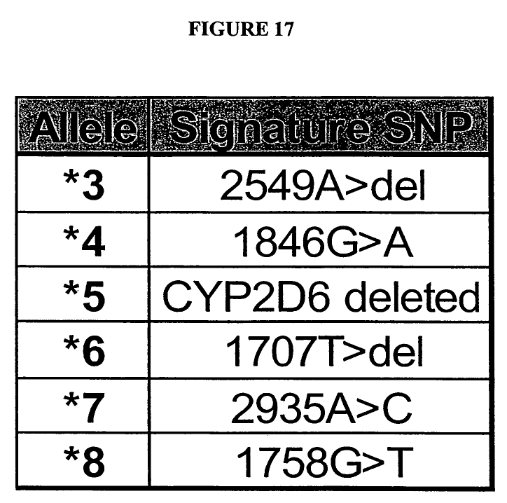

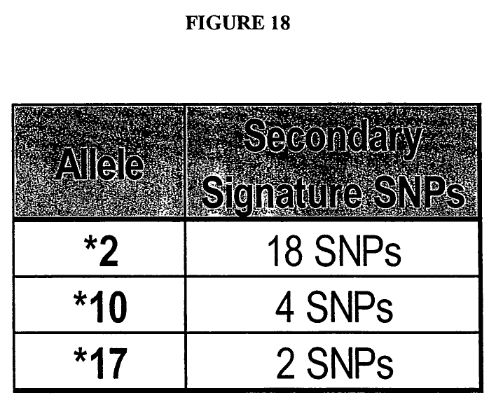

In the CYP2D6 gene, an allele is defined as a group of genetically linked SNP's, or a haplotype of SNP's. A single allele can be comprised of more than 1 SNP, and the same SNP can be present in more than one allele. Currently, there are 43 known CYP2D6 alleles, termed alleles 1-43; these 43 alleles are comprised of over 80 different SNP's. Many of these SNP's have little or no frequency data, and many of these SNPs do not cause a change in CYP2D6 phenotype. Given the complexity of making a CYP2D6 allele call, certain SNPs have been defined as “Signature SNP” for a particular allele if that SNP is found in a given allele only and not in any other alleles. Some alleles have only one signature SNP. Some alleles have more than one signature SNP. Some alleles do not have any signature SNPs. FIG. 17 provides an example of some of the star alleles with a signature SNPs. “Secondary Signature SNPs” are the minimum number of SNPs to necessarily to sufficiently discriminate one allele from any other alleles. Characteristics of the Secondary Signature SNPs may include but are not limited to; a group of at least two SNPs: SNP's that are not necessarily present in the allele; SNP's that can be Signature SNPs for other alleles. FIG. 18 shows examples of some of the alleles with an exemplary Secondary Signature SNPs. By creating a matrix of Signature SNPs and Secondary Signature SNPs, the haplotypes or alleles present in a particular sample can be determined.

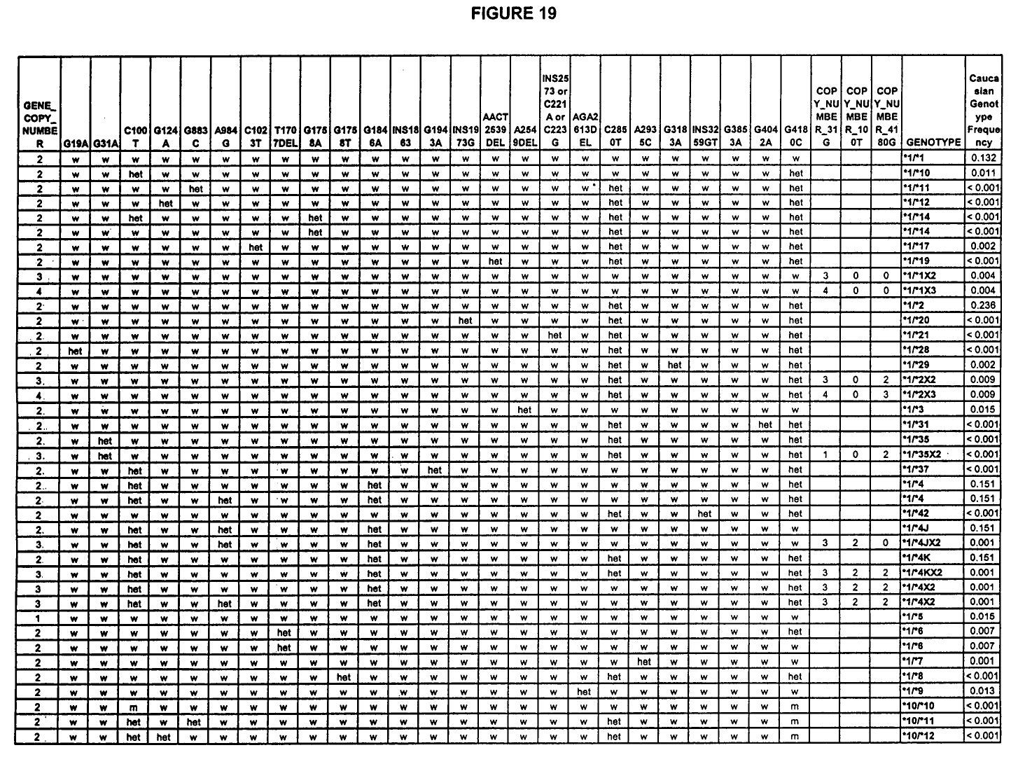

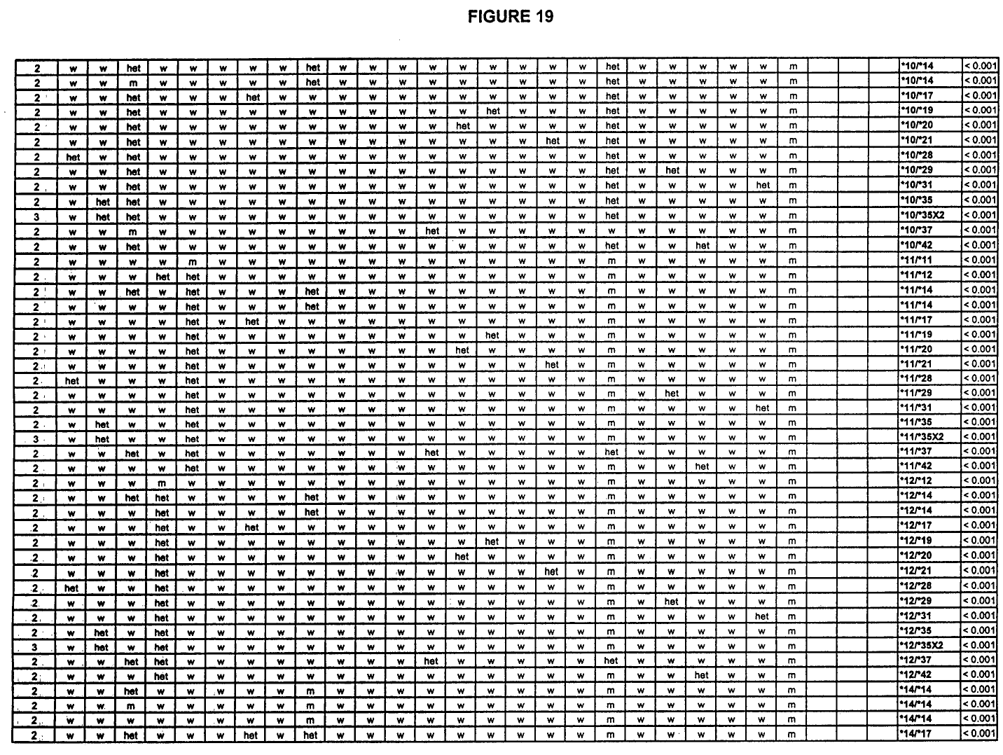

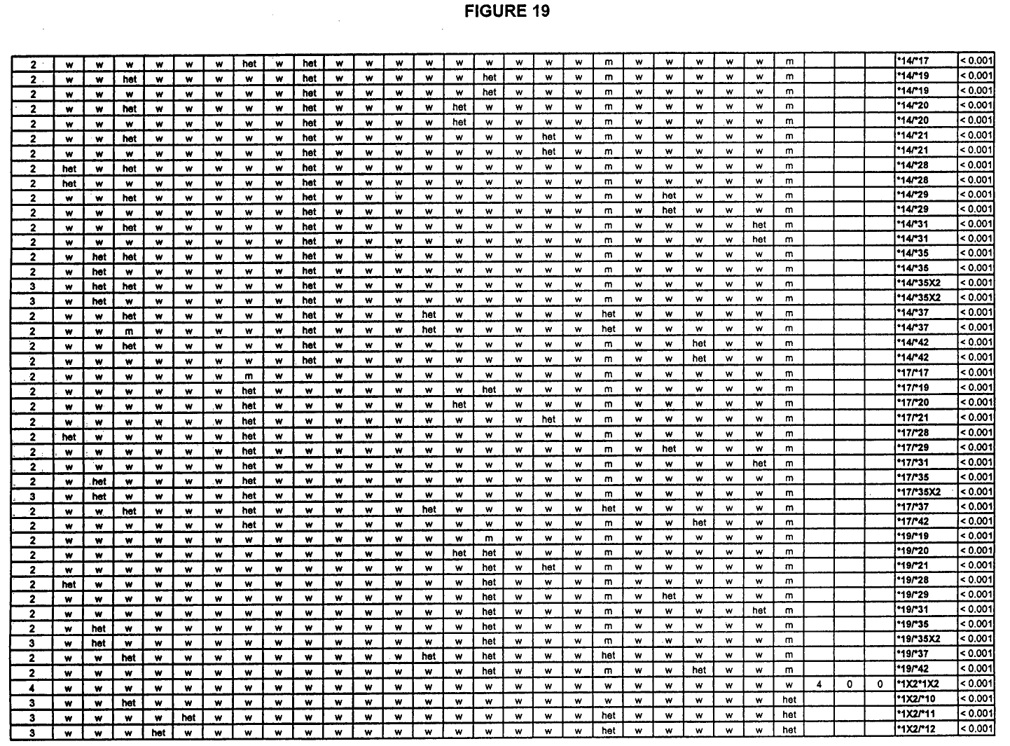

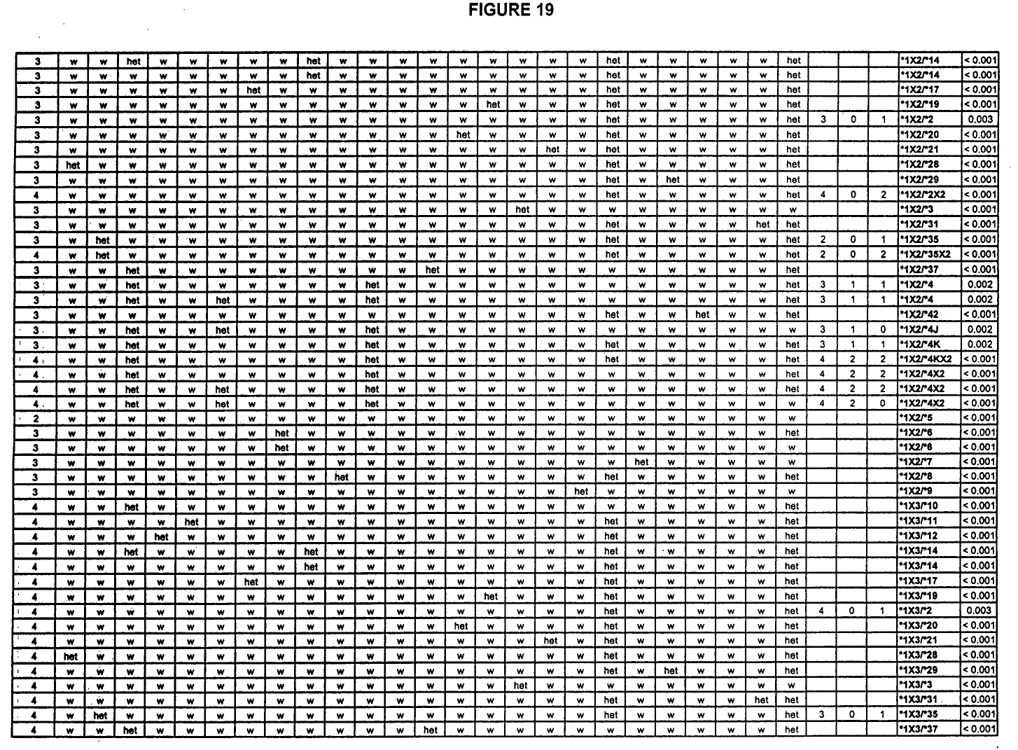

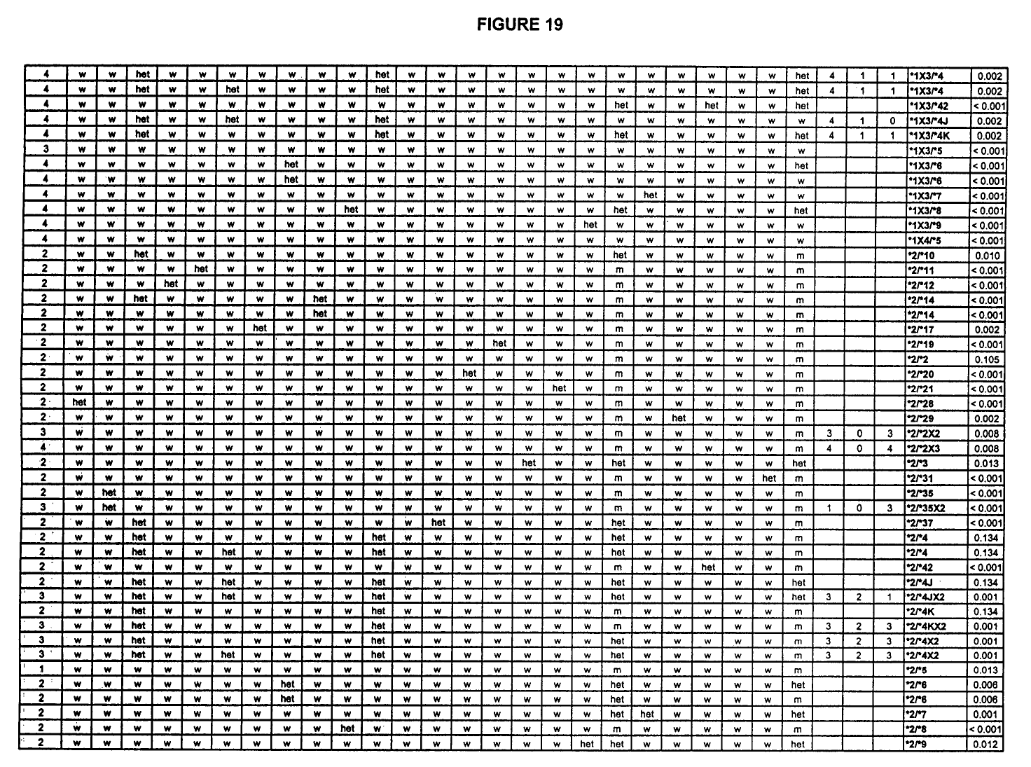

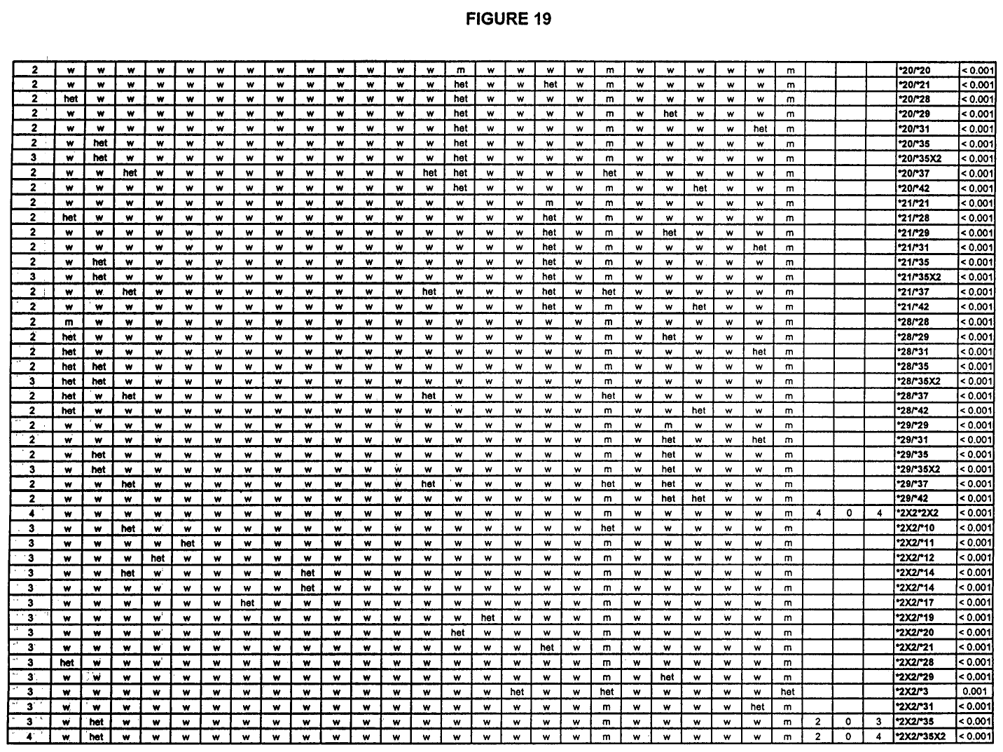

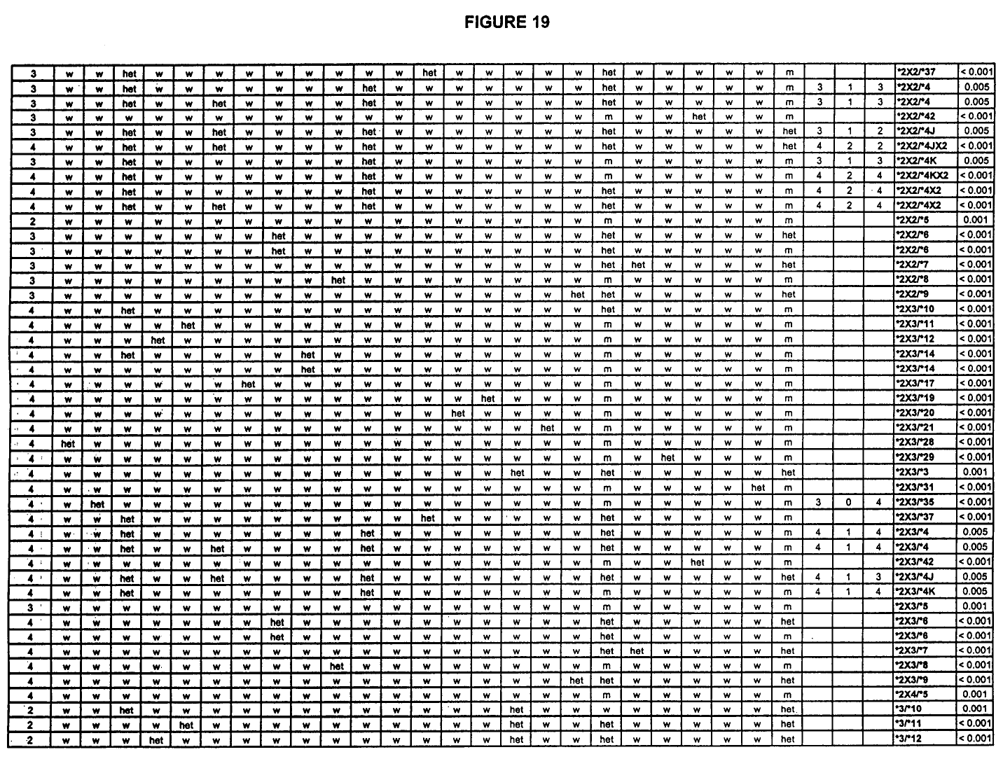

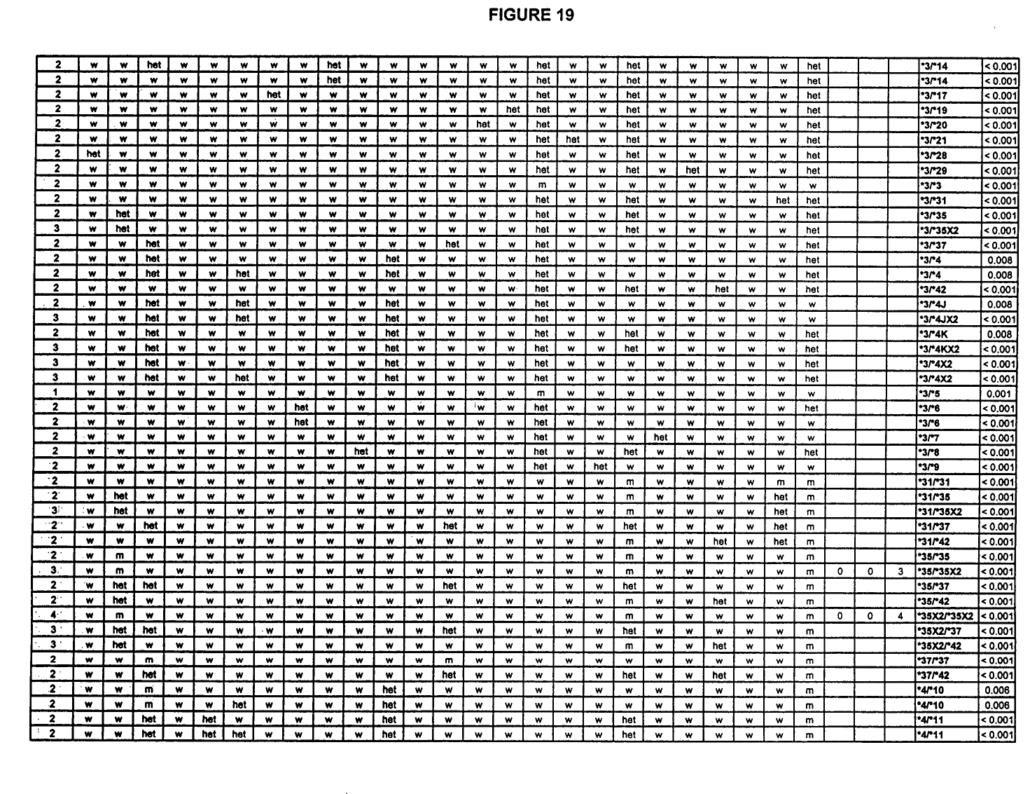

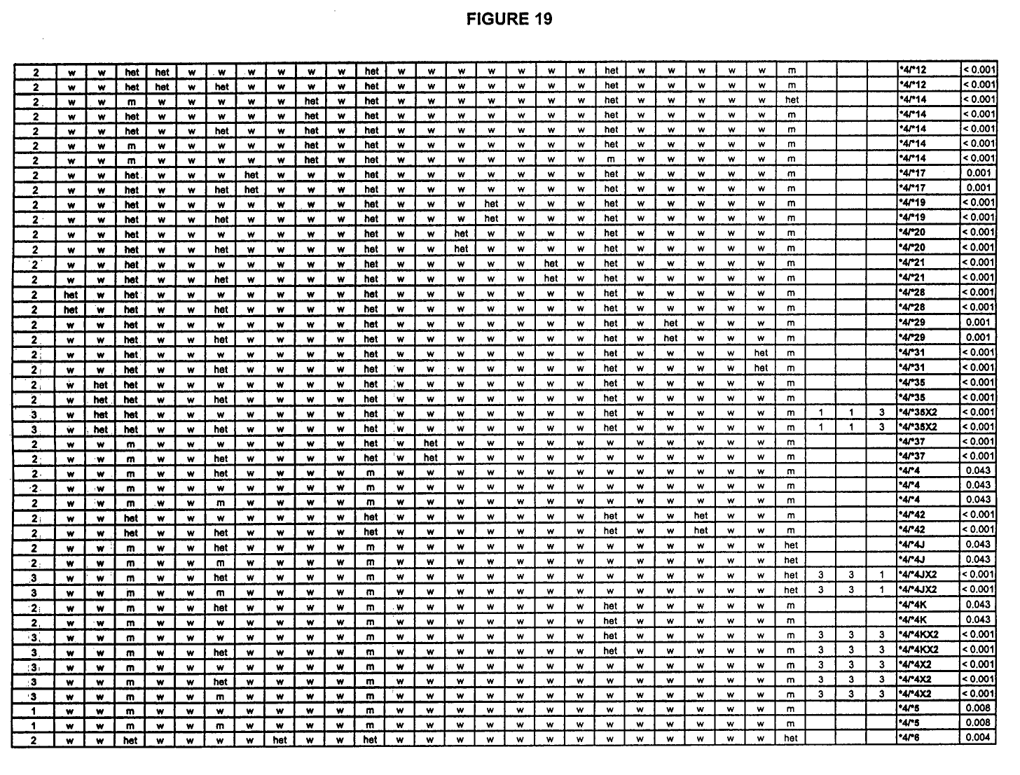

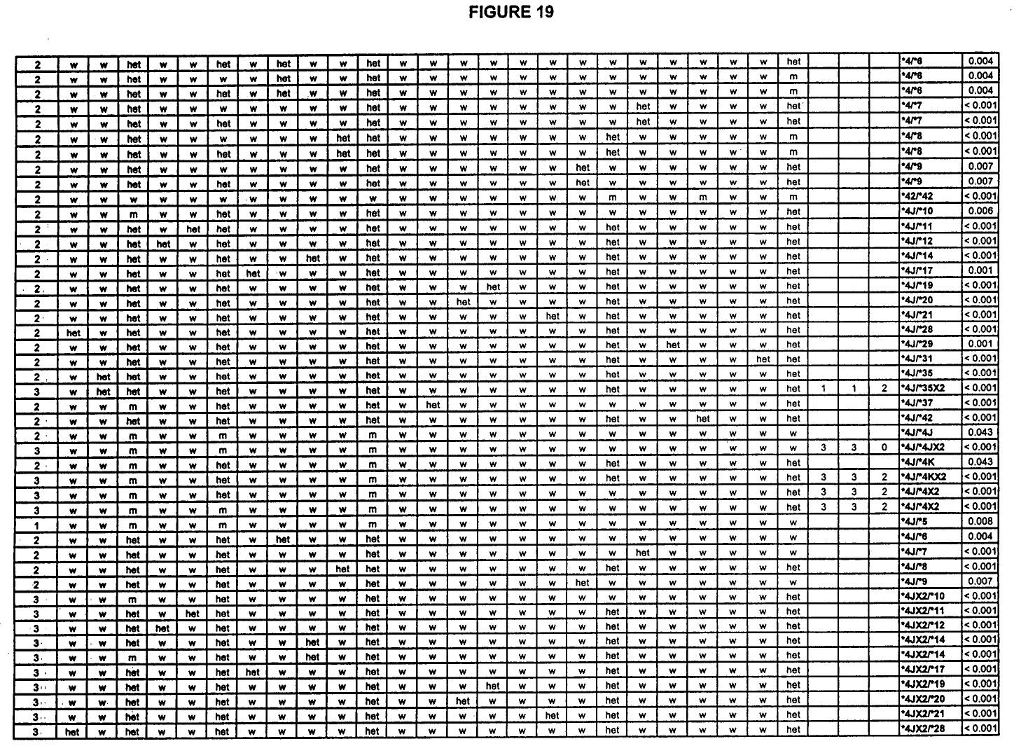

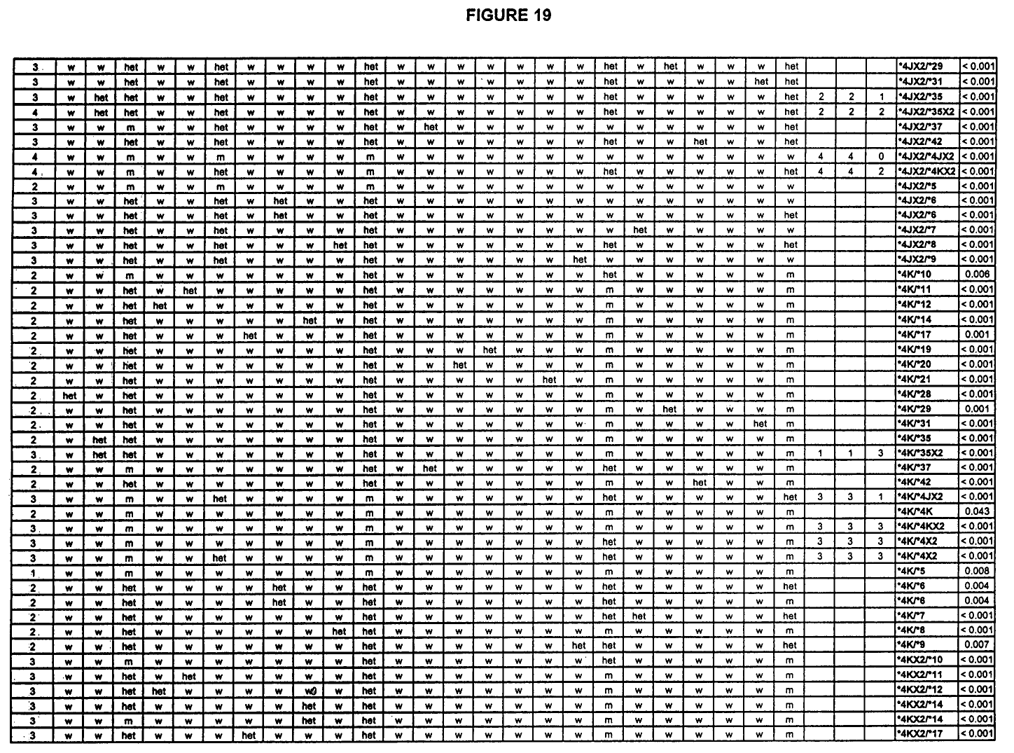

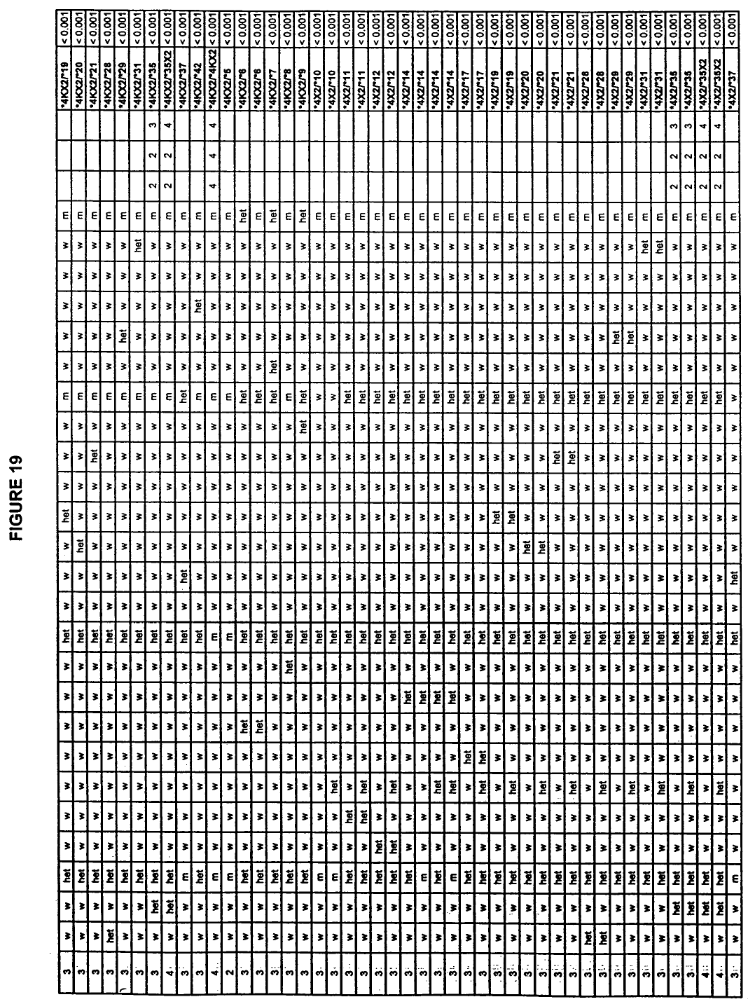

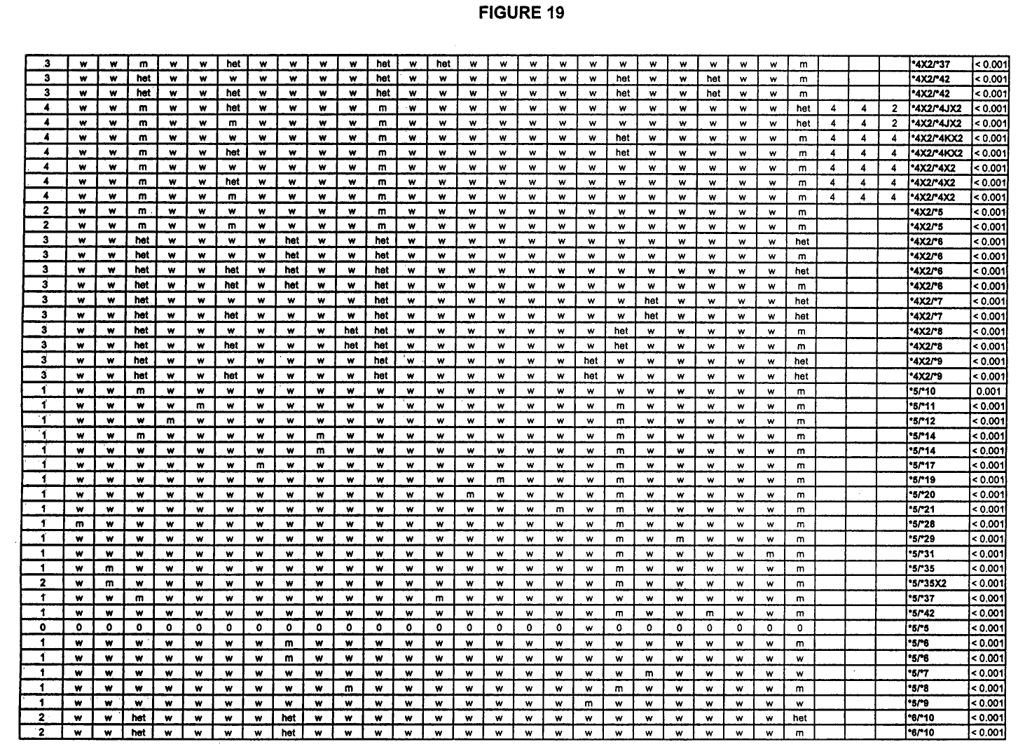

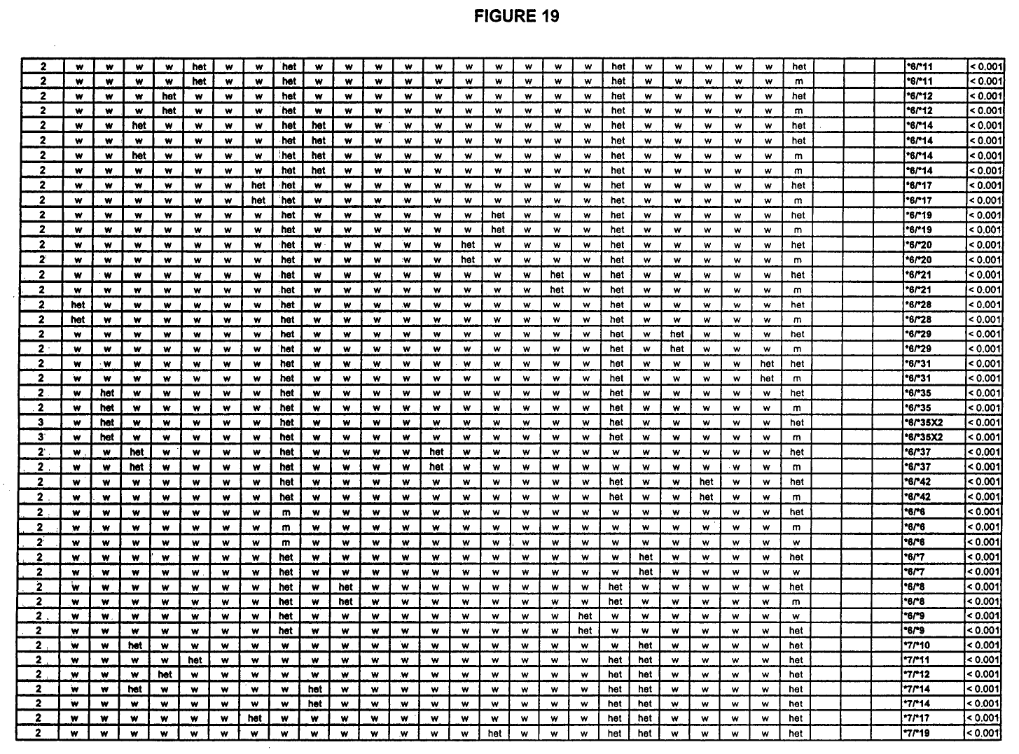

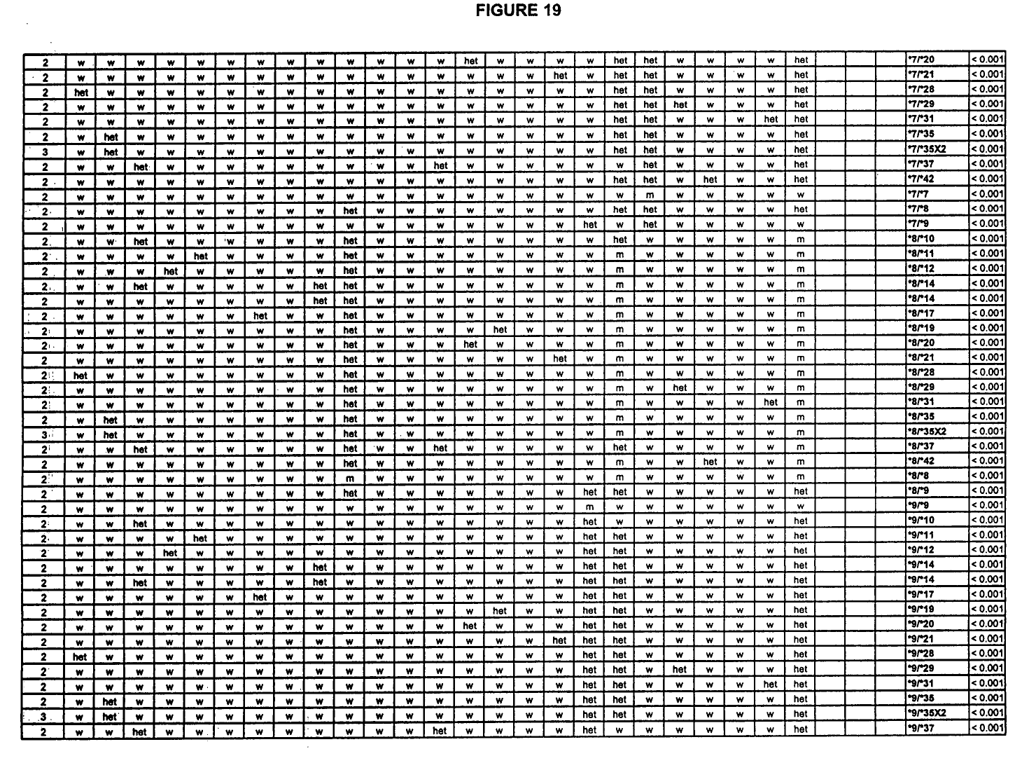

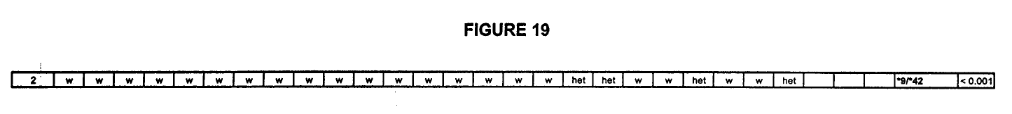

A set of different assays was selected (19G>A, 31G>A, 100C>T, 124G>A, 221C>A, 833G>C, 984A>G, 1023C>T, 1039C>T, 1661G>C, 1707T>del, 1758G>A, 1758G>T, 1846G>A, 1863ins[TTTCGCCCC]2, 1943G>A, 1973insG, 2539-2542delAACT, 2549A>del, 2613-2615delAGA, 2850C>T, 2935A>C, 3183G>A, 3259insGT, 3853G>A, 3887T>C, 4042G>A, 4180G>C, gene copy number, copy number 31G, copy number 100T, copy number 4180G) that represent 100% of the ultra-metabolizer phenotype and over 95% of the intermediate and poor metabolizer phenotypes in the Caucasian population. In another preferred embodiment, a further set of different assays was selected (19G>A, 31G>A, 100C>T, 124G>A, 833G>C, 984A>G, 1023C>T, 1707T>del, 1758G>A, 1758G>T, 1846G>A, 1863ins[TTTCGCCCC]2, 1943G>A, 1973insG, 2539-2542delAACT, 2549A>del, 2573insC, 2613-2615delAGA, 2850C>T, 2935A>C, 3183G>A, 3259insGT, 3853G>A, 4042G>A, 4180G>C, gene copy number). FIG. 19 provides an exemplary matrix representing all the possible combinations of all 29 assays and the full genotype of a sample carrying any one of these combinations. Thus, by comparing the experimental results of patient samples with the matrix of FIG. 19, a patient genotype is determined.

In some preferred embodiments, the assay detects not only the number of CYP2D6 copies present in a sample, but also distinguishes each allele. For example, the most commonly duplicated alleles are *1, *2, *4 and *35; each may yield a different phenotype.
*1: extensive metabolizer
*2: extensive metabolizer
*4: poor metabolizer
*35: extensive metabolizer

There is value in knowing not only how many copies of the gene are present, but also which alleles; different alleles in different ratios or combinations may yield different phenotypes. For example:
1/*2×n=ultra-rapid metabolizer
1×n/*2=ultra-rapid metabolizer
*1/*4×n=poor metabolizer

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A kit comprising an oligonucleotide detection assay configured for detecting the number of CYP2D6 gene copies present in a sample and configured to identify the presence or absence of at least two CYP2D6 associated polymorphisms.

2. The kit of claim 1, wherein said detection assay comprises an invasive cleavage assay.

3. The kit of claim 1, wherein said detection assay is configured to detect the copy of number of the CYP2D6 gene and, separately, the copy number of a least one portion of the CYP2D6 gene.

4. The kit of claim 1, wherein said CYP2D6 associated polymorphisms are selected from the group consisting of 19G>A, 31G>A, 100C>T, 124G>A, 221C>A, 833G>C, 984A>G, 1023C>T, 1039C>T, 1661G>C, 1707T>del, 1758G>A, 1758G>T, 1846G>A, 1863ins[TTTCGCCCC]2, 1943G>A, 1973insG, 2539-2542delAACT, 2549A>del, 2573insC, 2613-2615delAGA, 2850C>T, 2935A>C, 3183G>A, 3259insGT, 3853G>A, 3887T>C, 4042G>A, 4180G>C, gene copy number, copy number 31G, copy number 100T, and copy number 4180G.

5. The kit of claim 1, further comprising a control reagent for assessing CYP2D6 copy number.

6. The kit of claim 5, wherein said control reagent comprises reagents for detection of alpha-actin.

7. The kit of claim 5, wherein said control reagent comprises synthetic target nucleic acids having 0, 1, 2, 3, or 4 copies of a CYP2D6 gene sequence.

8. The kit of claim 1, wherein said detection assay is configured to detect the copy number of at least one of said polymorphisms.

9. The kit of claim 1, wherein said polymorphisms are selected from the group consisting of 31G>A, 100C>T, and 4180 G>C.

10. The kit of claim 5, wherein said control reagent comprises synthetic target nucleic acids having 0, 1, 2, 3, or 4 copies of a mutant CYP2D6 sequence.

11. A method for detecting a CYP2D6 genotype of a sample, comprising:
a) providing:
i) a sample comprising a target nucleic acid;
ii) a detection assay configured to detect at least two CYP2D6 polymorphic sequences and to detect CYP2D6 copy number;
b) exposing said sample to said detection assay under conditions such that said at least two CYP2D6 polymorphic sequences are detected and CYP2D6 copy number is detected, thereby detecting a CYP2D6 genotype of said sample.

12. The method of claim 11, wherein said detection assay comprises an invasive cleavage assay.

13. The method of claim 11, wherein said target nucleic acid is amplified prior to said exposure step.

14. The method of claim 11, wherein said detection assay is configured to detect the copy of number of the CYP2D6 gene and, separately, the copy number of a least one portion of the CYP2D6 gene.

15. The method of claim 11, wherein said CYP2D6 polymorphic sequences are selected from the group consisting of 19G>A, 31G>A, 100C>T, 124G>A, 221C>A, 833G>C, 984A>G, 1023C>T, 1039C>T, 1661G>C, 1707T>del, 1758G>A, 1758G>T, 1846G>A, 1863ins[TTTCGCCCC]2, 1943G>A, 1973insG, 2539-2542delAACT, 2549A>del, 2573insC, 2613-2615delAGA, 2850C>T, 2935A>C, 3183G>A, 3259insGT, 3853G>A, 3887T>C, 4042G>A, 4180G>C, gene copy number, copy number 31G, copy number 100T, and copy number 4180G.

16. The method of claim 11, wherein said detection assay further detects a copy number of at least one of said polymorphic sequences.

17. The method of claim 11, wherein said polymorphic sequences are selected from the group consisting of 31G>A, 100C>T, and 4180G>C.

18. A method for genotyping a subject having a CYP2D6 gene comprising the steps of:
a) detecting at least 25 single nucleotide polymorphisms associated with the CYP2D6 gene in said subject;
b) detecting the CYP2D6 gene copy number;
c) if multi-copy number polymorphisms are present, detecting the copy number of said multi-copy number polymorphism; and
d) generating a genotype profile based on the information derived from steps a-c; and
e) comparing said genotype profile to a predetermined CYP2D6 information matrix, such that a CYP2D6 genotype of said subject is determined.

19. The method of claim 18, wherein said single nucleotide polymorphisms and said information matrix is selected such that over 99% of Caucasian ultra metabolizers and over 95% of intermediate and low metabolizer are genotyped for CYP2D6.

20. The method of claim 18, wherein said 25 polymorphisms are selected from the group consisting of 19G>A, 31G>A, 100C>T, 124G>A, 221C>A, 833G>C, 984A>G, 1023C>T, 1039C>T, 1661G>C, 1707T>del, 1758G>A, 1758G>T, 1846G>A, 1863ins[TTTCGCCCC]2, 1943G>A, 1973insG, 2539-2542delAACT, 2549A>del, 2573insC, 2613-2615delAGA, 2850C>T, 2935A>C, 3183G>A, 3259insGT, 3853G>A, 3887T>C, 4042G>A, 4180G>C, gene copy number, copy number 31G, copy number 100T, and copy number 4180G.

21. The method of claim 18, wherein said multi-copy number polymorphisms are selected from the group consisting of 31G>A, 100C>T, and 4180G>C.

22. The method of claim 18, wherein said predetermined CYP2D6 information matrix is stored in a computer memory.

23. The method of claim 18, further comprising the step of using said CYP2D6 genotype in selecting a therapy for a subject.

24. The method of claim 18, further comprising the step of comparing said CYP2D6 genotype to a drug interaction observed in said subject.
